SEQUENCE LISTING (121 sequences)

SEQ ID NO: 1 - DNA, 4101 bp, C. glutamicum NRRL B-11474
aaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccgagccaagagggattgctggcgcacttcggtgcaattgctgcagcaacagaggttccaatttgtctctatgacattcctggtcggtcaggtattccaattgaatctgataccatgagacgcctgagtgaattacctacgattttggcggtcaaggacgccaagggtgacctcgttgcagccacgtcattgatcaaagaaacgggacttgcctggtattcaggcgatgacccactaaaccttgtttggcttgctttgggcggatcaggtttcatttccgtaattggacatgcagcccccacagcattacgtgagttgtacacaagcttcgaggaaggcgacctcgtccgtgcgcgggaaatcaacgccaaactatcaccgctggtagctgcccaaggtcgcttgggtggagtcagcttggcaaaagctgctctgcgtctgcagggcatcaacgtaggagatcctcgacttccaattatggctccaaatgagcaggaacttgaggctctccgagaagacatgaaaaaagctggagttctataaatatgaatgattcccgaaatcgcggccggaaggttacccgcaaggcgggcccaccagaagctggtcaggaaaaccatctggatacccctgtctttcaggcaccagatgcttcctctaaccagagcgctgtaaaagctgagaccgccggaaacgacaatcgggatgctgcgcaaggtgctcaaggatcccaagattctcagggttcccagaacgctcaaggttcccagaaccgcgagtccggaaataacaaccgcaaccgttccaacaacaaccgtcgcggtggtcgtggacgtcgtggatccggaaacgccaatgagggcgcgaacaacaacagtggtaaccagaaccgtcagggcggaaaccgtggcaaccgcggtggcggacgccgaaacgttgttaagtcgatgcagggtgcggatctgacccagcgcctgccagagccaccaaaggcaccggcaaacggtctgcgtatttacgcacttggtggcatttccgaaatcggtcgcaacatgaccgtgtttgagtacaacaaccgtctgctcatcgtggactgtggtgtgctcttcccatcttcaggtgagccaggcgttgacctgattcttcctgacttcggcccaattgaggatcacctgcaccgcgtcgatgcattggtggttactcacggacacgaagaccacattggtgctattccctggctgctgaagctgcgcaacgatatcccaattttggcatcccgtttcaccttggctctgattgcagctaagtgtaaggaacaccgtcagcgtccgaagctgatcgaggtcaacgagcagtccaatgaggaccgcggaccgttcaacattcgcttctgggctgttaaccactccatcccagactgccttggtcttgctatcaagactcctgctggtttggtcatccacaccggtgacatcaagctggatcagactcctcctgatggacgcccaactgacctgcctgcattgtcccgtttcggtgacgagggcgtggacttgatgctgtgtgactccaccaacgccaccacccctggtgtttctggatctgaagctgatgttgctccaaccctgaagcgtttggtcggcgatgctaagcagcgcgtcattttggcgtcgtttgcttccaacgtgtaccgcgttcaggcagctgtggatgctgctgttgcgtccaaccgtaaggttgcattcaacggtcgttccatgattcgcaacatggagatcgcggagaagcttggttacctgaaggcacctcgcggaaccattatttccatggatgatgcttctcgtatggctcctcacaaggtcatgctgattaccactggtactcagggtgagcctatggctgcgctgtctcgcatggcgcgtcgtgagcaccgacagatcactgtccgtgatggagacttgattatcctttcttcctccctggttccaggtaacgaagaagcagtgttcggtgtcatcaacatgctggctcagatcggtgcaactgttgttaccggtcgcgacgccaaggtgcacacctcgggccacggctactccggagagctgttgttcttgtacaacgccgctcgtccgaagaacgctatgcctgtccacggcgagtggcgccacctgcgcgccaacaaggaactggctatctccactggtgttaaccgcgacaacgttgtgcttgcacaaaacggtgttgtggttgatatggtcaacggtcgcgcacaggttgttggtcagattccagttggtaacctttacgtcgacggtgtcaccatgggtgatattgacgcggacatccttgcagaccgtacctcgcttggtgagggtggcttgatctcgatcactgctgtcatcgacaaccgcactggccgtctgttggagcgcccaaccgttcagaccagcggtttctctgaggatgcaaagtccatgatgggtgaggtcactgagctgtccgaaaccaccatgaatgatcttgcagctgaaggtgaaaacgatccttaccgcatggttcagcagctgcgccgcaagctctctcgcttcgtcgagcagaagtggaagcgccagccggtcatcatgccaaccgtcattccgatgactgcggaaaccacgcacatcggtgacgatgaggttcgcgcttcacgcgagtccctgtaaaagcatttcgcttttcgacgtcgcgagcccgcagcacccaacctgggtgccgcgggctcaacgcgtttctacacattgatctagtcttggttgcatgaatctcgcacaaacagaacgagcagcacttcaagcatcactgttggaaaaggggccagacgcaccaaccttgtgcggcgaatggaccaccaaagatcttgcggcacacctttatgtccgtgagcagaagccaggagcagcgctgcgcagtatgcttcccggcaagcaggatcctgcaaaagaagagtttgaagaagcgcttacccgcccttacgtggacgttgtcaacgcgtgggctgaaggcccacaggggctgaatccatggcgagcccttgactctattggaaatggcatggagcatttcattcaccatgaagatgttctgcgtggcgcgttgcagccaggcgatgaagtagaagttcgtcccatggaccatgcgcacggcaaagagctgcaccgcattttaaaacttctggctccacgaatgatcaaatcaccgcgacccgtggttctagctccggtgggttttccgaggattgttcttcatgaaggtcggggagtgtcggccgatggtgaaaacgtctgtcggattagcggtggggtaggggagctgctgttgtggctgtctggccgtgatgttgtgaaactaacatttgaaggcaaccaagagggcgttgttcggggaagtttatagaaaacttggacgaatcgcgtggtgcttctgtgaatagagttgtttgagcgactagagttaaggccatgactgtcagaaacggcgcacccaagcggggaagctcacgatcatcgggaaagtcgacggggccaaaaaccaccgcgacgaccaggcgaagcaaaccaacaaccagcatctctcggggtggccggtcagagactgctgcgtttacaacatcgatgaccgctcgacgcgctgctgatacttccgaagaacggaccggcagcgcatttcgcgctgtgggatccggtatcggaacgctatttggtgccaccgcgagtggcttgggtaagatgacccgttccattggtgaacttggtcgtcgtcgaaacgaagatgtactcgacgattttgatgatttcgaggaggagatcgcaacgaaacccgccacacgaaaatcgcggagcaaggctgttgaacctgagccggaattcgatgaggattttgatgagcaatcaggaaaccg

SEQ ID NO: 2 - DNA, 2157 bp, Artificial Sequence
  coding sequence for altered RnaJ protein
atgaatgattcccgaaatcgcggccggaaggttacccgcaaggcgggcccaccagaagctggtcaggaaaaccatctggatacccctgtctttcaggcaccagatgcttcctctaaccagagcgctgtaaaagctgagaccgccggaaacgacaatcgggatgctgcgcaaggtgctcaaggatcccaagattctcagggttcccagaacgctcaaggttcccagaaccgcgagtccggaaataacaaccgcaaccgttccaacaacaaccgtcgcggtggtcgtggacgtcgtggatccggaaacgccaatgagggcgcgaacaacaacagtggtaaccagaaccgtcagggcggaaaccgtggcaaccgcggtggcggacgccgaaacgttgttaagtcgatgcagggtgcggatctgacccagcgcctgccagagccaccaaaggcaccggcaaacggtctgcgtatttacgcacttggtggcatttccgaaatcggtcgcaacatgaccgtgtttgagtacaacaaccgtctgctcatcgtggactgtggtgtgctcttcccatcttcaggtgagccaggcgttgacctgattcttcctgacttcggcccaattgaggatcacctgcaccgcgtcgatgcattggtggttactcacggacacgaagaccacattggtgctattccctggctgctgaagctgcgcaacgatatcccaattttggcatcccgtttcaccttggctctgattgcagctaagtgtaaggaacaccgtcagcgtccgaagctgatcgaggtcaacgagcagtccaatgaggaccgcggaccgttcaacattcgcttctgggctgttaaccactccatcccagactgccttggtcttgctatcaagactcctgctggtttggtcatccacaccggtgacatcaagctggatcagactcctcctgatggacgcccaactgacctgcctgcattgtcccgtttcggtgacgagggcgtggacttgatgctgtgtgactccaccaacgccaccacccctggtgtttctggatctgaagctgatgttgctccaaccctgaagcgtttggtcggcgatgctaagcagcgcgtcattttggcgtcgtttgcttccaacgtgtaccgcgttcaggcagctgtggatgctgctgttgcgtccaaccgtaaggttgcattcaacggtcgttccatgattcgcaacatggagatcgcggagaagcttggttacctgaaggcacctcgcggaaccattatttccatggatgatgcttctcgtatggctcctcacaaggtcatgctgattaccactggtactcagagtgagcctatggctgcgctgtctcgcatggcgcgtcgtgagcaccgacagatcactgtccgtgatggagacttgattatcctttcttcctccctggttccaggtaacgaagaagcagtgttcggtgtcatcaacatgctggctcagatcggtgcaactgttgttaccggtcgcgacgccaaggtgcacacctcgggccacggctactccggagagctgttgttcttgtacaacgccgctcgtccgaagaacgctatgcctgtccacggcgagtggcgccacctgcgcgccaacaaggaactggctatctccactggtgttaaccgcgacaacgttgtgcttgcacaaaacggtgttgtggttgatatggtcaacggtcgcgcacaggttgttggtcagattccagttggtaacctttacgtcgacggtgtcaccatgggtgatattgacgcggacatccttgcagaccgtacctcgcttggtgagggtggcttgatctcgatcactgctgtcatcgacaaccgcactggccgtctgttggagcgcccaaccgttcagaccagcggtttctctgaggatgcaaagtccatgatgggtgaggtcactgagctgtccgaaaccaccatgaatgatcttgcagctgaaggtgaaaacgatccttaccgcatggttcagcagctgcgccgcaagctctctcgcttcgtcgagcagaagtggaagcgccagccggtcatcatgccaaccgtcattccgatgactgcggaaaccacgcacatcggtgacgatgaggttcgcgcttcacgcgagtccctgtaa

SEQ ID NO: 3 - protein, 718 aa, Artificial Sequence
  altered RnaJ protein
MNDSRNRGRKVTRKAGPPEAGQENHLDTPVFQAPDASSNQSAVKAETAGNDNRDAAQGAQGSQDSQGSQNAQGSQNRESGNNNRNRSNNNRRGGRGRRGSGNANEGANNNSGNQNRQGGNRGNRGGGRRNVVKSMQGADLTQRLPEPPKAPANGLRIYALGGISEIGRNMTVFEYNNRLLIVDCGVLFPSSGEPGVDLILPDFGPIEDHLHRVDALVVTHGHEDHIGAIPWLLKLRNDIPILASRFTLALIAAKCKEHRQRPKLIEVNEQSNEDRGPFNIRFWAVNHSIPDCLGLAIKTPAGLVIHTGDIKLDQTPPDGRPTDLPALSRFGDEGVDLMLCDSTNATTPGVSGSEADVAPTLKRLVGDAKQRVILASFASNVYRVQAAVDAAVASNRKVAFNGRSMIRNMEIAEKLGYLKAPRGTIISMDDASRMAPHKVMLITTGTQSEPMAALSRMARREHRQITVRDGDLIILSSSLVPGNEEAVFGVINMLAQIGATVVTGRDAKVHTSGHGYSGELLFLYNAARPKNAMPVHGEWRHLRANKELAISTGVNRDNVVLAQNGVVVDMVNGRAQVVGQIPVGNLYVDGVTMGDIDADILADRTSLGEGGLISITAVIDNRTGRLLERPTVQTSGFSEDAKSMMGEVTELSETTMNDLAAEGENDPYRMVQQLRRKLSRFVEQKWKRQPVIMPTVIPMTAETTHIGDDEVRASRESL

SEQ ID NO: 4 - DNA, 4101 bp, C. glutamicum NRRL B-11474
gctgcagacaggtagtcacctgcgatagccagaccgttctgggttccggagaaggaagcaccaccggtgtagaagtcggtggattcgctggtgctcttgcccacgcgaagcaccacggtcatcgtcacaataatgaatacgacgaagacactgatgttaagaatcggattaccgacgccctcagaaacagcgtcttgtgcaaggagaatagtggaattcatatctgattaaccttccatcttctggcgaatagcagcctgacgaggctccaagttcctgttcgcaaacatgacgtagacgtaagtaataatgaatgtggttacgaactgaaggaagccgaagattaggccgatgttgattgcgccgaaaactggggttgccatccactctgatgcaaaggatgcaaccagaacgtagacgacgtaccacaagaagaaggcaacggtcattgggaaagcaaaggaacggaacttgctgcggagctctccaaattctgggctcgcctgcatgtcccgaaactcttgagccgtcggctggcgacgcccctgggtgatttgttctgcattcactggaaacttatctcgctttcatatccctcgagcgggagtcggtgatcggccactctttaagcaatgccggctttaaaataaagcaacttatatgtttctcaccacatctggccgacgaccgctaagtatgttgtcgatcacagctaaacgtgtgaatgtgaagttacctaactcacattgcaatgcgatagcgatttggaaaactcactccccccaataccttaacttgaacttaaaaatagtggtttacctgcatttataaaagttcccgatctaccccctctttaccccgaaataccccttttgcaaagattgcaaacacaacagtgcaatagttaacgggcttcacacgtcacctttctgtccggttttaggctatgttcgggacgtctaggcaaaagtagttttgtgagatgaaacgcataatccgtcattttttacgcaatcgatagcctaaattgggcttagatcttccgcctctaaataggtatgcaaagacattcgaattaattaacaaagccatttttcggccgtggagaagcgttttccgactatggtgtggggcatggaacacacttcagcattgacgctcatagactcggttttggaccctgacagcttcatttcttgggatgaaactccccaatatgacaacctcaatcaaggctatgcagagaccttggagcgggctcgaagcaaggccaaatgcgatgaatcggtgattactggagaaggcaccgtggagggcattccggtagccgttattttgtccgatttttccttcctcggcggttctttgggcacggtcgcgtcggtgcgcatcatgaaggcgattcaccgcgccacagagctgaaactcccactgctggtctcccctgcttccggtggtgcgcgcatgcaggaagacaatcgagcctttgtcatgatggtgtccataaccgcggctgtgcaacgccaccgcgaagcacatttgccgttcctggtgtatttgcgcaatcccacgatgggtggcgccatggcctcgtggggttcatctgggcatctcacttttgcggaacccggcgcgcagatcggtttcctgggccctcgcgtggtggagttgaccaccgggcgtgcgcttccagacggtgtgcagcaggcggagaatttggtgaaaactggtgtgattgatggcattgtgtcgccgctccaattgcgtgcagcggtggcaaaaacgctcaagattattcagcctgcagaggcaaccgatcgtttttctccaacaactcctggcgcagcgcttccggtgatggaggcgattgcgcgttctcgtgacccgcacaggcctggaatcggggagattatggaaacgttgggggcagacgtcgtcaggctatctggtgcgcgtgctggcgcgttgagctcggctgtgcgcgttgccctagcgcgcatcgggggccggcctgtggtgctgattggacaggatcggcgcttcacgcttgggccgcaggagctgcgttttgcgcgccgtggcatttcgttggcgcgcgagctgaacctgccgatcgtgtccatcatcgacacctccggcgccgaattgtcacaggcggctgaggagctcggcatcgcaagctcgattgcgcgcaccttgtccaagcttatcgacgctcccctccccaccgtttcggtcattattggtcagggcgttggcggtggcgcgctggccatgctgcccgccgatctggtctacgcggccgaaaacgcgtggctgtccgcattgcctccagagggcgcctcggccatcctcttccgcgacaccaaccacgccgcggaaatcatagagcgacaaggcgtgcaggcgcacgcacttttaagccaagggcttatcgacgggatcgtcgccgaaaccgagcactttgttgaagaaattctcggcacaatcagcaacgccctctccgaattggataacaatccggagagggcaggacgcgacagtcgcttcacacgatttgagcgtttagcgcagtaaagaaaattatgcgctgatcaaatcgatgatgaacaccagggtacggccagacagtgggtggccggaaccctcagggccgtaagcagcctctggcggaatggtcagctgacgacgtccgccgaccttcatgcctggaattccttcttgccaacctgcaatgaggccgttgagtgggaactggctggtctgtccacgatcccaggaagagtcaaactcctcgccggtttcaaggtcaacgcccacatagtggacctcaacttctccgcctgggcgggcttctgcgccttcgccaacgatgatgtcagagattacgagatcttccggtgctggaccgaccggtagctcaatctgtggcttttccatgtgctgtacatgctccttgaaaatcgtattttttggtttcatgcaaaaacggccgaaaccatccctgtcattatgacagacgaagtggcttcggccgaagttgaacgagaatcatccgctaaatttagcgctcctcgcgaggaaccaacttgcgggattccttgccggtgtagacctggcgtgggcggttgatcttgttgcctgctgcaccgagctgctcgcggtagtgagcgatccatcctggcagacgaccgattgcgaacaatacggtgaagaagtcagttgggaagcccattgcgcggtagatcaggccggtgtagaagtctacgttcgggtagagcttgcgggagatgaagtaatcatcagccagtgcaatttcttccagcttgattgccagatccagaagatcgtcgccaccgaggtgctcgaggatctcgtgtgcggtctccttgacgattgctgcacgtggatcgtagttcttgtaaacgcggtgtccgaagcccatgaggcggacgccgtcttccttgttcttgaccttgttcatgaacgcggttgcgtcgccaccgtggttgttcttgatgtcttcgagcatctccagaacagcctggtttgcgccaccgtgcagtgggccggacagagcgttgatgccaccagcgatggagacaaacatgttggcctgtgcggaaccgatcatacgaacggtggaggtggagcagttctgctcgtggtcagcgtgcaggatgagcagcttgtccagagccttgaccatgattgggtcgatctcgtatggctcggttgggtaaccgaacatcatgcgcaggaagttctcacgcgcgttgagggagttgtctgggtacatgtaaggagcacccttgcgtgcgcggtgtgcgtacgcagccagcattggaacctttgccatgaggcgaacggttgccttatcaagctgtgcctcatcgagtgggttcagctgatcctggtagtaggtagacaaaatgttaaccgaggaagccaaggttgccattgggtgagcgtcgcgtgggaacacgttgaactgggacttgaagtcctcgtccagaagggtgtggtggcgaatctcgtcgttaaacttgtgaagctcatctggggttggtagctcaccgttgataagtaggtaagaaacct

SEQ ID NO: 5 - DNA, 1455 bp, Artificial Sequence
  coding sequence for altered accDA protein
atggtgtggggcatggaacacacttcagcattgacgctcatagactcggttttggaccctgacagcttcatttcttgggatgaaactccccaatatgacaacctcaatcaaggctatgcagagaccttggagcgggctcgaagcaaggccaaatgcgatgaatcggtgattactggagaaggcaccgtggagggcattccggtagccgttattttgtccgatttttccttcctcggcggttctttgggcacggtcgcgtcggtgcgcatcatgaaggcgattcaccgcgccacagagctgaaactcccactgctggtctcccctgcttccggtggtgcgcgcatgcaggaagacaatcgagcctttgtcatgatggtgtccataaccgcggctgtgcaacgccaccgcgaagcacatttgccgttcctggtgtatttgcgcaatcccacgatgggtggcgccatggcctcgtggggttcatctgggcatctcacttttgcggaacccggcgcgcagatcggtttcctgggccctcgcgtggtggagttgaccaccgggcgtgcgcttccagacggtgtgcagcaggcggagaatttggtgaaaactggtgtgattgatggcattgtgtcgccgctccaattgcgtgcagcggtggcaaaaacgctcaagattattcagcctgcagaggcaaccgatcgtttttctccaacaactcctggcgcagcgcttccggtgatggaggcgattgcgcgttctcgtgacccgcacaggcctggaatcggggagattatggaaacgttgggggcagacgtcgtcaggctatctggtgcgcgtgctggcgcgttgagctcggctgtgcgcgttgccctagcgcgcatcgggggccggcctgtggtgctgattggacaggatcggcgcttcacgcttgagccgcaggagctgcgttttgcgcgccgtggcatttcgttggcgcgcgagctgaacctgccgatcgtgtccatcatcgacacctccggcgccgaattgtcacaggcggctgaggagctcggcatcgcaagctcgattgcgcgcaccttgtccaagcttatcgacgctcccctccccaccgtttcggtcattattggtcagggcgttggcggtggcgcgctggccatgctgcccgccgatctggtctacgcggccgaaaacgcgtggctgtccgcattgcctccagagggcgcctcggccatcctcttccgcgacaccaaccacgccgcggaaatcatagagcgacaaggcgtgcaggcgcacgcacttttaagccaagggcttatcgacgggatcgtcgccgaaaccgagcactttgttgaagaaattctcggcacaatcagcaacgccctctccgaattggataacaatccggagagggcaggacgcgacagtcgcttcacacgatttgagcgtttagcgcagtaa

SEQ ID NO: 6 - protein, 484 aa, Artificial Sequence
  altered accDA protein
MVWGMEHTSALTLIDSVLDPDSFISWDETPQYDNLNQGYAETLERARSKAKCDESVITGEGTVEGIPVAVILSDFSFLGGSLGTVASVRIMKAIHRATELKLPLLVSPASGGARMQEDNRAFVMMVSITAAVQRHREAHLPFLVYLRNPTMGGAMASWGSSGHLTFAEPGAQIGFLGPRVVELTTGRALPDGVQQAENLVKTGVIDGIVSPLQLRAAVAKTLKIIQPAEATDRFSPTTPGAALPVMEAIARSRDPHRPGIGEIMETLGADVVRLSGARAGALSSAVRVALARIGGRPVVLIGQDRRFTLEPQELRFARRGISLARELNLPIVSIIDTSGAELSQAAEELGIASSIARTLSKLIDAPLPTVSVIIGQGVGGGALAMLPADLVYAAENAWLSALPPEGASAILFRDTNHAAEIIERQGVQAHALLSQGLIDGIVAETEHFVEEILGTISNALSELDNNPERAGRDSRFTRFERLAQ

SEQ ID NO: 7 - DNA, 4067 bp, C. glutamicum NRRL B-11474
cacctgcgacaacaccgagaaccgcgaggaagagcaccacgcgcatgaggccagtgatcaggatgccggtgatagaggaattggcaacagctttgacgctgttggggccggttcgtccggagtccagcatgcggtgtgcgccagcgtaagtgatgtatccaccgacggttccacccacaagtgtggtgatgacaagccagtcgatggtgtcaggaagtactgcattcttcagcgccgagccaactggaggttgggagacgaaagccacgtagacggtgagcaggatcatcacgacgccgaggaccacgaggaatttgtccagagcagcaccaagtcgcttgaacaagaagatcgcaatcgcgatggccgcggtgatcacgccaccaactttgacgtcccagccaagcagagcgttaagcccaaggccaccaccggcgatgttgccgatgttgaatactatgccgccaatacagaccagtacggccagcacccaaccaaaacctgggataacagtattgccgagttcttgggcgcgcatttcagagatgccgatgatgcgccacacattcagctgcaccgcgatgtcaatgaggatcgacaccaggatcgcaaatgcgaaagctgcgccgagctggttggtgaagacagcggtttgggtgaggaaccctgggccgatggcagaagttgccatgaggaagatggcacccatcagagcgcttcggccagaggcggttactgcagcttgcttgagggatggcgcattggcttttgcaatgtcgccaggcccaggggttccagtgctggctgagactagctggctgtcatcgacatttttgttgtcggccattgtcgcgctcctttcgtggggggctttcaaaatgattggggtagcttgcacaccaatcgcagattgttgaacaatcttggtgagttgagagtgagtttagacccaatgtgtcctgtggcacaatcccccatagggattaatagtcattgcatacggccagaccacccccgcaagtgcctaggtgtgtccccagtcctttaagatcagtcacatgacgcaggcaatagcagcatcccttgatttagcggctcgaatcaccgccaaaattgatcaaggagtgctcactccaggtactcgactacccgaggttgctttggcagaagaacttggcgtttcacggaacacgctgcgtgaagcttttcgggtactcatgcaagacggactggtggatcatattcccaaccgtggggttttcgtgcacacgttcaccaagtcggatgtggaagatatctatgcttaccgcacatttatcgaggttgctgcgattaggtcggcgcggaaaaatcctcagttgctggagcagtctttgggggtaatgcgagaggcctacgaaaggggtgctgcagccaatgccgtgggtgattggcaaactgtcggttctgccaacagtgcttttcacttggcgattgtggacctagcaggagtggctaggttgtcagcagacgctcgaaaagtgttggcgctggctcgcatcggattcatggccacctacaacgtggagacattccatagcatttacgtggaaaagaaccatcaaatcttgaagtatttggctgccggtgaattcgaagaggcggaacaatacctccagaaatacttcgaagattcgcgcgatgatttgtctgcgcacctaccggaattttgaaattgcgatagcatagatgacttgacactgttgcagcaggcggttttcacctggcctcttaggatttggtcaaatggttgcaacgacgccagggaaggaggcgcaccccatggcatttggattttttagtagacgtaagaaaaagaacaaagacaaaaacccgaatgaaaattcagcagtgcccgcacactctgaagattcacctcaggaggtttttgagggtaatggtcgtcaggtaggcgaccccattgaacagcaggttgatcgagatgctaaaggtcgtctcacagcggcggatttcttgccggacgctgatctgccacagctgaatcgttcgcgtgcaaatatgctgcgccgtgaattggagtaccgtttttcactccagaatgcccacattaatatcgatggaaacacggccatgattcagcgttcagatggcggggcagcacatgtctcgttgcgcaccctcgcgatgaatgcagctggccttgataactttgatcaactccctgaactggtggaaagcttcgttcacggcacgctggccgatgcaacattaaacgatctttctactgctgacctgtataaagcactgcgccttcgcctgctgccaacacctggtgaaggcgacgatctagttgagcatggactcgaccgggaaagccagatccgcgacgattcaatcctgcgcaccttcacctctgacatgtcgatcgcgctggtgctcgataccgagcatgccatccgcatccagccactcaaagagctcgaggagttcgatgacctcagcgccctagagcgggctgcggaccgcaatacctggcaagagctttacgacgcaaacgttgacgcttccttcgtcgacgctgaatcagacagcgaagggtcatcattttgggctttcgaatctaactcgtactacctgggtagtgcaccactgttcctcaacgatttgttggcaaagtgggcacctgacctggaccaaagtgatggcgtcatctttgctgtccctgatcgtgatctgttgattgcgcgtcctgtgaccaccggcgaagatctgatgaacggaatcaccgcgatggtgaggatcgcgatgcgctttggcctcgggaacccgacgtcgataagcccgcgcctgcacctgctgcgcgacaaccaggtgaccaccttcaccgacttccgcgtcgtctctcctgaaatggaagctgaatgggaagacagcgcgtttgacgcgccaccggccggcgcgatcggcattgaggtgcgcccagatccgtatctgatggagcgcctccaacagggcggctttggtgatttcggagatttcggcaagccccgcgatctagatatgtagcgaaaaaaggaccttcacttcttaagaagtgaaggtccttatttatcgggttctagtgttagaacttgttctcgcgctcggtgtgagccatagcaagaacgtcgaggcgcttgtccagctcttcctcggtgagcttctcgccatcaaccaagcccaaatcgatgacagtctggcggatggtcttgccctcagccaaagcagtcttagccacctttgcagcagcttcgtagccgattgcagagttcagtggggtaacgatggaaggtgaagactcagcgagctccttcatgtgtgcctcgtttggctcaatgccatcaacgagacgggttgcgaacacgcgggaagtgttagccagcaggcgagcggactcaagcacgttgcgagccatcactgggatgaacacgttgagctcgaactggccctgggtgccggagaacgcaacagctgcgtcattgccgataacctgagcggaaacctgggtagcggtctcacagagaactgggttgaccttgcctggcatgatggaggaacccggctgcaggtctgggagacggatctcgccaagaccggtcagtgggccggagcccatgaggcggatatcgttagcgatcttgtacaaggagacagcgataacgcgcattgcgccggagaactcaacaagagcgtcgcgtgcagcctgagcctcgaagtggttctcagcttccttgagctccttgacgtcggtcaagttgatcagttcagcaacaaccttgccgccgaaatcagcggaggtgttgataccggtaccagcagcggtgccaccaatagccagctcaccaaggcgaggaagagtagcctcaacgcgctcgatgccgagctggatctggcgagcgtagccaccgaactcctggcccagggttactggaacagcgtccatcaggtgggtgcggccggacttaacaacctcagaccactcgttagccttcttcgccaaagactcgtgcagaactttcaggcctggg

SEQ ID NO: 8 - DNA, 1245 bp, Artificial Sequence
  coding sequence for altered cg1144 protein
atggcatttggattttttagtagacgtaagaaaaagaacaaagacaaaaacccgaatgaaaattcagcagtgcccgcacactctgaagattcacctcaggaggtttttgagggtaatggtcgtcaggtaggcgaccccattgaacagcaggttgatcgagatgctaaaggtcgtctcacagcggcggatttcttgtcggacgctgatctgccacagctgaatcgttcgcgtgcaaatatgctgcgccgtgaattggagtaccgtttttcactccagaatgcccacattaatatcgatggaaacacggccatgattcagcgttcagatggcggggcagcacatgtctcgttgcgcaccctcgcgatgaatgcagctggccttgataactttgatcaactccctgaactggtggaaagcttcgttcacggcacgctggccgatgcaacattaaacgatctttctactgctgacctgtataaagcactgcgccttcgcctgctgccaacacctggtgaaggcgacgatctagttgagcatggactcgaccgggaaagccagatccgcgacgattcaatcctgcgcaccttcacctctgacatgtcgatcgcgctggtgctcgataccgagcatgccatccgcatccagccactcaaagagctcgaggagttcgatgacctcagcgccctagagcgggctgcggaccgcaatacctggcaagagctttacgacgcaaacgttgacgcttccttcgtcgacgctgaatcagacagcgaagggtcatcattttgggctttcgaatctaactcgtactacctgggtagtgcaccactgttcctcaacgatttgttggcaaagtgggcacctgacctggaccaaagtgatggcgtcatctttgctgtccctgatcgtgatctgttgattgcgcgtcctgtgaccaccggcgaagatctgatgaacggaatcaccgcgatggtgaggatcgcgatgcgctttggcctcgggaacccgacgtcgataagcccgcgcctgcacctgctgcgcgacaaccaggtgaccaccttcaccgacttccgcgtcgtctctcctgaaatggaagctgaatgggaagacagcgcgtttgacgcgccaccggccggcgcgatcggcattgaggtgcgcccagatccgtatctgatggagcgcctccaacagggcggctttggtgatttcggagatttcggcaagccccgcgatctagatatgtag

SEQ ID NO: 9 - protein, 414 aa, Artificial Sequence
  altered cg1144 protein
MAFGFFSRRKKKNKDKNPNENSAVPAHSEDSPQEVFEGNGRQVGDPIEQQVDRDAKGRLTAADFLSDADLPQLNRSRANMLRRELEYRFSLQNAHINIDGNTAMIQRSDGGAAHVSLRTLAMNAAGLDNFDQLPELVESFVHGTLADATLNDLSTADLYKALRLRLLPTPGEGDDLVEHGLDRESQIRDDSILRTFTSDMSIALVLDTEHAIRIQPLKELEEFDDLSALERAADRNTWQELYDANVDASFVDAESDSEGSSFWAFESNSYYLGSAPLFLNDLLAKWAPDLDQSDGVIFAVPDRDLLIARPVTTGEDLMNGITAMVRIAMRFGLGNPTSISPRLHLLRDNQVTTFTDFRVVSPEMEAEWEDSAFDAPPAGAIGIEVRPDPYLMERLQQGGFGDFGDFGKPRDLDM

SEQ ID NO: 10 - DNA, 4210 bp, C. glutamicum NRRL B-11474
cgcagtcatccggtggagaagacttctcgtggtacctcgaacacgtcccaggatcaatggcccggttgggttgctggccggggcacggacccaagcaagacctccatcaaagtgacctggttgtggatgagcgagccatcggagttggcgtcaggctctttggctcccttgtgcagcagtacagtagccgatctgaagctttcttaaattcctaatgggggtagtgtgtagggctggctctaaattgctgacacgtcacccaggctaattcagcagtaatcatttagacttggaatcgcttaccagtggtttcaacaatgcattcacccagctcacacgtgtggaggtgccttaatggcaaagaggatcgtaattatcggcggtggacctgcaggctatgaagccgcactcgcaggcgctaaatacggtgcagaagttaccgttattgaagatgtcggagttggcggatccgcagtcaccatggactgtgtaccttcaaagtccttcatcgctggtaccggtatcaaaaccgacctccgacgtgctgatgacatgggacttaaccgtgggcttggaaaagcacacctagaaatcgatgcactgaacatccgtgtgaaggaccttgcgaaagcacagtccgaagatatcttgggccagctgcagcgctcagatgtccgcatgattaacggtgtgggccgctttgatgattacaacaccaagcaaaccacccactacattaaagtcacccacagcgatggctccgaagaaaccattgagtgcgatctggtgctggttgcaactggtgcaaccccccgcattcttaaaggtgcagagccagacggcgagcgcatcttgacctggcgtcaggtctatgacattgaagaactccccacccaccttatcgtggttggttccggtgtgaccggtgcggaatttgtctctgcgtttgctgaactcggcgtcaaagtcaccatggtggcatcccgtgaccgcattttgcctcacgatgacgcagatgccgcagacgtgctggaaaccgttctggctgagcgcggagtatccctggaaaagcatgcccgcgtggagtctgtcacccgcaccgaagacggtggcgtgtgtgttcgcactgctgacggacgtgaaatctacggttctcacgcgttgatgactgttggttccattccaaacacggcagatcttggcctggagaacatcggtgttgagctggcaccatccggccatatcaaggttgaccgcgtctcccgcaccaacatccccggtgtgtacgcagcaggtgactgtactgacctattcccactggcgtccgttgcagcgatgcagggccgtatcgccatgtatcacgcactcggtgaaggcgtgagccccatccgtttgaagactgttgccaccgcagtgtttacccgcccagagatcgcagcagtaggtatcacccatgcacaggttgattccggcgaagtgtctgctcgcgtgattgtgcttcctttggctactaacccacgcgccaagatgcgttccctgcgccacggttttgtgaagctgttctgccgccgtaactctggcctgatcatcggtggtgtcgtggtggcaccgaccgcgtctgagctgatcctaccgatcgctgtggcagtgaccaaccgtctgacagttgctgatctggctgataccttcgcggtgtacccatcattgtcaggttcgattactgaagcagcacgtcagctggttcaacatgatgatctaggctaatttttctgagtctcagattttaagaaaacccaggattgctttgtgcactcctgggttttcactttgttaagcagttttggggaaaagtgcaaagtttgcaaagtttagaaatattttaagaggtaagatgtctgcaggtggaagcgtttaaatgcgttaaacttggccaaatgtggcaacctttgcaaggtgaaaaactggggcggggttagatcctggggggtttatttcattcactttggcttgaagtcgtgcaggtcaggggagtgttgcccgaaaacattgagaggaaaacaaaaaccgatgtttgattgggggaatcgggggttacgatactaggacgaagtgactgctatcacccttggcggtctcttgttgaaaggaataattactctagtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtactgaaggctcaccagtcaaggcgtacctggacatcgatgaaattatcggtgcagctaaaaaagttaaagcagatgctatttacccgggatatggcttcctgtctgaaaatgcccagcttgcccgcgagtgcgcggaaaacggcattacttttattggcccaaccccagaggttcttgatctcaccggtgataagtctcgtgcggtaaccgccgcgaagaaggctggtctgccagttttggcggaatccaccccgagcaaaaacatcgatgacatcgttaaaagcgctgaaggccagacttaccccatctttgtaaaggcagttgccggtggtggcggacgcggtatgcgctttgtttcttcacctgatgagctccgcaaattggcaacagaagcatctcgtgaagctgaagcggcattcggcgacggttcggtatatgtcgaacgtgctgtgattaacccccagcacattgaagtgcagatccttggcgatcgcactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgttttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggatttatcggcgatcacccacacctccttcaggctccacctgcggatgatgagcagggacgcatcctggattacttggcagatgtcaccgtgaacaagcctcatggtgtgcgtccaaaggatgttgcagcaccaatcgataagctgcccaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccacctccggcgtggacatcttccgcatcttcgacgcgcttaacgacgtctcccagatgcgtccagcaatcgacgcagtcctggagaccaacaccgcggtagccgaggtggctatggcttattctggtgatctctctg

SEQ ID NO: 11 - DNA, 93 bp, Artificial Sequence
  promoter 1860
cttagctttgacctgcacaaatagttgcaaattgtcccacatacacataaagtagcttgcgtatttaaaattatgaacctaaggggtttagca

SEQ ID NO: 12 - DNA, 3516 bp, Artificial Sequence
  promoter 1860 and pyc coding sequence
cttagctttgacctgcacaaatagttgcaaattgtcccacatacacataaagtagcttgcgtatttaaaattatgaacctaaggggtttagcagtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtactgaaggctcaccagtcaaggcgtacctggacatcgatgaaattatcggtgcagctaaaaaagttaaagcagatgctatttacccgggatatggcttcctgtctgaaaatgcccagcttgcccgcgagtgcgcggaaaacggcattacttttattggcccaaccccagaggttcttgatctcaccggtgataagtctcgtgcggtaaccgccgcgaagaaggctggtctgccagttttggcggaatccaccccgagcaaaaacatcgatgacatcgttaaaagcgctgaaggccagacttaccccatctttgtaaaggcagttgccggtggtggcggacgcggtatgcgctttgtttcttcacctgatgagctccgcaaattggcaacagaagcatctcgtgaagctgaagcggcattcggcgacggttcggtatatgtcgaacgtgctgtgattaacccccagcacattgaagtgcagatccttggcgatcgcactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgttttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggatttatcggcgatcacccacacctccttcaggctccacctgcggatgatgagcagggacgcatcctggattacttggcagatgtcaccgtgaacaagcctcatggtgtgcgtccaaaggatgttgcagcaccaatcgataagctgcccaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccacctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacattctggccattaaggatatggctggtctgcttcgcccagctgcggtaaccaagctggtcaccgcactgcgccgtgaattcgatctgccagtgcacgtgcacacccacgacactgcgggtggccagttggctacctactttgctgcagctcaagctggtgcagatgctgttgacggtgcttccgcaccactgtctggcaccacctcccagccatccctgtctgccattgttgctgcattcgcgcacacccgtcgcgataccggtttgagcctcgaggctgtttctgacctcgagccgtactgggaagctgtgcgcggactgtacctgccatttgagtctggaaccccaggcccaaccggtcgcgtctaccgccacgaaatcccaggcggacagttgtccaacctgcgtgcacaggccaccgcactgggccttgctgatcgcttcgagctcatcgaagacaactacgcagccgttaatgagatgctgggacgcccaaccaaggtcaccccatcctccaaggttgttggcgacctcgcactccacctggttggtgcgggtgtagatccagcagactttgctgcagacccacaaaagtacgacatcccagactctgtcatcgcgttcctgcgcggcgagcttggtaaccctccaggtggctggccagaaccactgcgcacccgcgcactggaaggccgctccgaaggcaaggcacctctgacggaagttcctgaggaagagcaggcgcacctcgacgctgatgattccaaggaacgtcgcaacagcctcaaccgcctgctgttcccgaagccaaccgaagagttcctcgagcaccgtcgccgcttcggcaacacctctgcgctggatgatcgtgaattcttctacggactggtcgagggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcactgtgactgttgctgaaggtgatgaggtcaaggctggagatgcagtcgcaatcatcgaggctatgaagatggaagcaacaatcactgcttctgttgacggcaagattgaacgcgttgtggttcctgctgcaacgaaggtggaaggtggcgacttgatcgtcgtcgtttcctaa

SEQ ID NO: 13 - DNA, 4101 bp, C. glutamicum NRRL B-11474
catcgtctaaaaggcctgggcgaccacgtaaagacgggcacgacgagaagatcatcgacgcaactttacggctcatcgacagcaatcgtcccgtcacggtcaatgcagttgtcaaagaaagcggagtggcacgtgcagcggtttatcgacgctggcccaggctagtggatctagtagcggaagctttagatgccgggcgagctccagttgaaatagataccccaggggacatcaaagagaccttgattgatgggctgtttacaaatcaggcgaaaaccactggagtctcctatcctcgtcagcgatttcgcaaacggctcgagttggtgatgtcagatcaagaattacagctcgcctaatggaattcacatgtgaagagacgtcgagaagcaaatattcgcgcgctgcaagtcgcgcaagaaaaaggccaaatccgggcggatctagacatcgaggcgtgcctcgatgcaatccttggggtgttttattaccaatcggtcgcgcgtggagtaaatttcaccgaccaaggtacaacgcaacgatgcagagaagccttggaggtgatctggcatggaatggaaccttaaattcaggttctgacgaggtgcgaagcaagttgtcgcgcgccgcacctcagtatccggatcaacttaatttcgaagtgctgggttttctcgcgcatacccaatgcgtaccgatgtgctcatgagcgaaaaacaggccacgataagtttcttaaaacttatcgtggcctgcttctatatttgtgcgccctgacgggctcgaaccgccgacctgctgggtgtaaaccagctgctcttccagctgagctaaaggcgcgcacgtgcttttctagaaccaccttggtggcctcgaaagcaacgagtgaaatactaacacacaatctccacagacctaaaatcgctgctcaggccgtggaaattagcgattgttaaggcttcttgtttccacgctggacgaggcaagaaccttgccaattaccgagacgttccgccttggtctgcacgagacctgccagttgtgctgattcagagataactccaggagccagggctccttctttaccaatgccaggagtcaacacccagatacgaccattctcagcgagggagcggatggaatccacaagtccgtcgacgagatcgccgtcatcctcgcgccaccagagcagcacgacatcgcacagctcgtcggtttcttcatcgagtagttcctcaccgattgcatcttcgatggactcgctgatcagcgtgtcggaatcttcatcccatccaatttcttgaacgatatgacccgattgaatgccgagtagttgagcataatcctgggcaccttgcttgactgcgcccggagcgtcggccactttaataatcctcctcgtgtgggccccgatgtgtttttcgattacatggattcaacatgaaaccgcggggctattgatatatccgaattgcacattaccgtccaaccggtactttgaaccacctttccctggaattttttccttttcctccccctttacgctcaagaatcaatgaattcaatcactggccagcgattaacttttcgagttttcagtcttggatttccacaattctcttcaaaataatggtggctagatttttcatcaaaccctcaccaaaaggacatcagacctgtagttttatgcgattcgcgtcaaacgtgagagaaacatcacatctcacgggaaactacccgataattctttgcaaaactttgcaaagggtaatgaacatgcagctagtttccgtagaaatgttctttaaaaaatccacaacaattgccaggaagcacaccgattgatggatacctgaaatcccagtgagcgcaccactccccttacgtcacagtctgtaaaacaaatcttcggtgttgcgtatccttgttaataacttatgcgttgacccattcgtgcacttcggtgtgccacaattaggtacgaccaagaatgggaccgggaaaccgggacgtataaacgaaataaaacattccaacaggaggtgtggaaatggccgatcaagcaaaacttggtggtaagccctcggatgactctaacttcgcgatgatccgcgatggcgtggcatcttatttgaacgactcagatccggaggagaccaacgagtggatggattcactcgacggattactccaggagtcttctccagaacgtgctcgttacctcatgcttcgtttgcttgagcgtgcatctgcaaagcgcgtatctcttcccccaatgacgtcaaccgactacgtcaacaccattccaacctctatggaacctgaattcccaggcgatgaggaaatggagaagcgttaccgtcgttggattcgctggaacgcagccatcatggttcaccgcgctcagcgaccaggcatcggcgtcggcggacacatttccacttacgcaggcgcagcccctctgtacgaagttggcttcaaccacttcttccgcggcaaggatcacccaggcggcggcgaccagatcttcttccagggccacgcatcaccaggtatgtacgcacgtgcattcatggagggtcgcctttctgaagacgatctcgatggcttccgtcaggaagtttcccgtgagcagggtggcattccgtcctaccctcacccacacggtatgaaggacttctgggagttcccaactgtgtccatgggtcttggcccaatggatgccatttaccaggcacgtttcaaccgctacctcgaaaaccgtggcatcaaggacacctctgaccagcacgtctgggccttccttggcgacggcgaaatggacgagccagaatcacgtggtctcatccagcaggctgcactgaacaacctggacaacctgaccttcgtggttaactgcaacctgcagcgtctcgacggacctgtccgcggtaacaccaagatcatccaggaactcgagtccttcttccgtggcgcaggctggtctgtgatcaaggttgtttggggtcgcgagtgggatgaacttctggagaaggaccaggatggtgcacttgttgagatcatgaacaacacctccgatggtgactaccagaccttcaaggctaacgacggcgcatatgttcgtgagcacttcttcggacgtgacccacgcaccgcaaagctcgttgagaacatgaccgacgaagaaatctggaagctgccacgtggcggccacgattaccgcaaggtttacgcagcctacaagcgagctcttgagaccaaggatcgcccaaccgtcatccttgctcacaccattaagggctacggactcggccacaacttcgaaggccgtaacgcaacccaccagatgaagaagctgacgcttgatgatctgaagttgttccgcgacaagcagggcatcccaatcaccgatgagcagctggagaaggatccttaccttcctccttactaccacccaggtgaagacgctcctgaaatcaagtacatgaaggaacgtcgcgcagcgctcggtggctacctgccagagcgtcgtgagaactacgatccaattcaggttccaccactggataagcttcgctctgtccgtaagggctccggcaagcagcagatcgctaccactatggcgactgttcgtaccttcaaggaactgatgcgcgataagggcttggctgatcgccttgtcccaatcattcctgatgaggcacgtaccttcggtcttgactcttggttcccaaccttgaagatctacaacccgcacggtcagaactacgtgcctgttgaccacgacctgatgctctcctaccgtgaggcacctgaaggacagatcctgcacgaaggcatcaacgaggctggttccgtggcatcgttcatcgctgcgggtacctcctacgccacccacggcaaggccatgattccgctgtacatcttctactcgatgttcggattccagcgcaccggtgactccatctgggcagcagccgatcagatggcacgtggcttcctcttgggcgctaccgcaggtcgcaccaccctgaccggtgaaggcctccagcacatggatggacactcccctgtcttggcttccaccaacgagggtgtcga

SEQ ID NO: 14 - DNA, 2051 bp, Artificial Sequence
  altered aceE coding sequence
ttggccgatcaagcaaaacttggtggtaagccctcggatgactctaacttcgcgatgatccgcgatggcgtggcatcttatttgaacgactcagatccggaggagaccaacgagtggatggattcactcgacggattactccaggagtcttctccagaacgtgctcgttacctcatgcttcgtttgcttgagcgtgcatctgcaaagcgcgtatctcttcccccaatgacgtcaaccgactacgtcaacaccattccaacctctatggaacctgaattcccaggcgatgaggaaatggagaagcgttaccgtcgttggattcgctggaacgcagccatcatggttcaccgcgctcagcgaccaggcatcggcgtcggcggacacatttccacttacgcaggcgcagcccctctgtacgaagttggcttcaaccacttcttccgcggcaaggatcacccaggcggcggcgaccagatcttcttccagggccacgcatcaccaggtatgtacgcacgtgcattcatggagggtcgcctttctgaagacgatctcgatggcttccgtcaggaagtttcccgtgagcagggtggcattccgtcctaccctcacccacacggtatgaaggacttctgggagttcccaactgtgtccatgggtcttggcccaatggatgccatttaccaggcacgtttcaaccgctacctcgaaaaccgtggcatcaaggacacctctgaccagcacgtctgggccttccttggcgacggcgaaatggacgagccagaatcacgtggtctcatccagcaggctgcactgaacaacctggacaacctgaccttcgtggttaactgcaacctgcagcgtctcgacggacctgtccgcggtaacaccaagatcatccaggaactcgagtccttcttccgtggcgcaggctggtctgtgatcaaggttgtttggggtcgcgagtgggatgaacttctggagaaggaccaggatggtgcacttgttgagatcatgaacaacacctccgatggtgactaccagaccttcaaggctaacgacggcgcatatgttcgtgagcacttcttcggacgtgacccacgcaccgcaaagctcgttgagaacatgaccgacgaagaaatctggaagctgccacgtggcggccacgattaccgcaaggtttacgcagcctacaagcgagctcttgagaccaaggatcgcccaaccgtcatccttgctcacaccattaagggctacggactcggccacaacttcgaaggccgtaacgcaacccaccagatgaagaagctgacgcttgatgatctgaagttgttccgcgacaagcagggcatcccaatcaccgatgagcagctggagaaggatccttaccttcctccttactaccacccaggtgaagacgctcctgaaatcaagtacatgaaggaacgtcgcgcagcgctcggtggctacctgccagagcgtcgtgagaactacgatccaattcaggttccaccactggataagcttcgctctgtccgtaagggctccggcaagcagcagatcgctaccactatggcgactgttcgtaccttcaaggaactgatgcgcgataagggcttggctgatcgccttgtcccaatcattcctgatgaggcacgtaccttcggtcttgactcttggttcccaaccttgaagatctacaacccgcacggtcagaactacgtgcctgttgaccacgacctgatgctctcctaccgtgaggcacctgaaggacagatcctgcacgaaggcatcaacgaggctggttccgtggcatcgttcatcgctgcgggtacctcctacgccacccacggcaaggccatgattccgctgtacatcttctactcgatgttcggattccagcgcaccggtgactccatctgggcagcagccgatcagatggcacgtggcttcctcttgggcgctaccgcaggtcgcaccaccctgaccggtgaaggcctccagcacatggatggacactcccctgtcttggcttccaccaacgagggtgtcga

SEQ ID NO: 15 - DNA, 4099 bp, C. glutamicum NRRL B-11474
gaatcttatcccatggctaagaaagtagacacctcgaacgctacccccgctctagcccttcttacggagaggcaaattccttttgagctggatgttcatgatgtagatccaaaatcatcaaagggcttcgcattggatgcctctgaagtaatgggtgtggagccggaagtggtgtttaaaacgctcatggcagatattgatggtgaacacgtggtcgcgattgttccagccagcagcacgttgaatctcaagcagttggctaaggctggaaaaggtaagcatgcaaacatgatggatcgcagccgtgcacaggtagtcacggggtatgtccctggtggaatctcaccgatagggcagaagaataagcaccgcgtgtttttggatgagtctgcaattctccaggagcgaatctacgtcagcgcaggacgccgaggctggtcactgattatcgccccggatgatgttcttctggctaccgatggggtttacgcggatattgctgatcattcataaaaggtaaaacccacccccgcaggggtgcgtcgtaaagcaagctccttttcttagtacggcgcgatagtggactcgtgcgcgtcgatgttgatgcggccgtgcacggacgggaatgcgcgctgcgcgcagttctcgcgggtgcacactcggcagccggacccgatgggggtggcggtggataggtcctggaggttgaagccgcgggagtacacggtgcggtcggcgtggcgcgcttcgcagccgagcccgatggcgaacattttgtccacttcgccgaaccgggcttcgtggtgtcgcacggtgcgtgagatccacaggtagttgcgtccgtcgggcatttgcgcgaattggcggagcacttggccggggttggtgaaggtttcaaacacgttccacagggggcaggtgccgccgtagtgggtgaagtggaagccggtggcggattggcgtttggacatgttgccggcgcggtcgacgcgcacgaaggtaaacgggatgccgcgcaggttgggacgctgcagggtggacaggcggtgggcggtcgtctcgtagcccacgccgaaaagctgacctaggtattcgatgtcgtagccggatttttcggcctcggagtggaagattttgtagggcagcattacggcggcggcgaagtaggaggcgacgccgcggatggctagggtgcgggcttcgggggtggaccagatgccgtcgtcgacgatgccttcgatgaggtcgttggcttctaggtagccgagttcggtggccatgcggaaggcgcgttgtccggggttgaggcgtgcgtggattgtcagtaggcgcgtctcggggtcaaagtggtgcagcgtgccggattcctctttggaggaggtgatggtgacatcgtgatccatttgcaggcgcctggcgatcgaatcttccatggcgcgggaatcgtacggctgccagcccagttgcgcggcgatggcttcggcgcggcggtcgagcgcatcgaagtagttttggcgggcgtaaatgaaatcgcgcacctcttcgtgcggcatgctcacggcctccgcgatggggcgacgttcctcaggcgtgttggtgcgattgtccactgcgatggagaacttgtcgcgtacgtttcggtagcgctggtgcatttccaccatggcgcgcgccagctgcgggtggttgtacaccatctccgatagctcttggagctccacgttcgcggggttgatctcccggtccaacatgacatcctgaacttcagctaaaaggcgggaatcatcgtcgcgggagaaaaacgttgcgtctacgccgaacgcctcggtgatgcgcaataacaccggtacggtgagcgggcgtacgtcgtgctcaatctgatttacataactggcagataagccaagggttgctgccaacgacgcctggctcaggtctctttcgcggcgcagttggcgcagcctggaccccacatatgtctttcccatggcgcaactatagcgtgatcaccatcaccttaaccactcttggcattgaggtgttgcaaagttgttgattttcgcttttcgacgcagcccgccgccatcgggtgcccggcgtggtcaggccacatgcgccccgggaactttttgggcacctacggtgcaacagttgcgaaaattgtgtcacctgcgcaaagccttgcttctattcgggaaattcgggtgtctaaactttttggttgataccaaacggggttagaaactgttcagatcggtatcctgtgaggaagctcaccttggttttagaatgttgaaaaagcctcacgtttccgcaggtagagcacactcaattaaatgagcgtcaaacgacaataaagtaaggctaccctaataactggggttttatgcctctaaacagtcagttgggggcggtaggggagcgtcccatgactggttaatgcctcgatctgggacgtacagtaacagcgacactggaggtgccatgactgttagaaatcccgaccgtgaggcaatccgtcacggaaaaattacgacggaggcgctgcgtgagcgtcccgcatacccgacctgggcaatgaagctgaccatggccatcactggcctaatcttcggtggcttcgttcttgttcacatgatcggaaacctgaaaatcttcatgccggactacgcagccgattctgcgcatccgggtgaagcacaagtagatgtctacggcgagttcctgcgtgagatcggatccccgatcctcccacacggctcagtcctctggatcctacgtattatcctgctggtcgcattggttctgcacatctactgtgcattcgcattgaccggccgttctcaccagtcccgcggaaagttccgccgtaccaacctcgttggcggcttcaactccttcgcgacccgctccatgctggtgaccggaatcgttctccttgcgttcattatcttccacatcctcgacctgaccatgggtgttgctccagcagccccaacttcattcgagcacggcgaagtatacgcaaatatggtggcttcctttagccgctggcctgtagcaatttggtacatcattgccaacctggtcctgttcgtccacctgtctcacggcatctggcttgcagtctctgacctgggaatcaccggacgtcgctggagggcaatcctcctcgcagttgcgtacatcgttcctgcactggtcctgatcggcaacatcaccattccgttcgccatcgctgttggctggattgcgtaaaggttaggaagaatttatgagcactcactctgaaaccacccgcccagagttcatccacccagtctcagtcctcccagaggtctcagctggtacggtccttgacgctgctgagccagcaggcgttcccaccaaagatatgtgggaataccaaaaagaccacatgaacctggtctccccactgaaccgacgcaagttccgcgtcctcgtcgttggcaccggcctgtccggtggcgctgcagcagcagccctcggcgaactcggatacgacgtcaaggcgttcacctaccacgacgcacctcgccgtgcgcactccattgctgcacagggtggcgttaactccgcccgcggcaagaaggtagacaacgacggcgcataccgccacgtcaaggacaccgtcaagggcggcgactaccgtggccgcgagtccgactgctggcgtctcgccgtcgagtccgtccgcgtcatcgaccacatgaacgccatcggtgcaccattcgcccgcgaatacggtggcgccttggcaacccgttccttcggtggtgtgcaggtctcccgtacctactacacccgtggacaaaccggacagcagctgcagctctccaccgcatccgcactacagcgccagatccacctcggctccgtagagatcttcacccataacgaaatggttgacgtaattgtcaccgaacgtaatggtgaaaagcgctgcgaaggcctgatcatgcgcaacctgatcaccggcgagctcaccgcacacaccggccatgccgttatcctggcaaccggtggctacggcaacgtgtaccacatgtccaccctggcgaagaactccaacgcctcg

SEQ ID NO: 16 - DNA, 1338 bp, Artificial Sequence
  codon-optimized lysA coding sequence
atggctaccgtagaaaacttcaatgaactacccgcgcatgtctggcctcgtaatgcagttcgccaggaagatggcgttgttacggtagcgggagtgccgcttcctgatcttgcagaagagtacggtacacctctctttgttgttgacgaagatgatttccgttctcgatgtcgtgatatggctaccgcatttgggggaccaggcaatgtgcattacgcttccaaagcgttccttactaaaactattgcacgctgggtagacgaagagggactagcgttggatatcgcgtctattaatgaactgggaattgcgttggcggctggattcccggcttcacgaatcacggcacatggtaataataaaggtgtcgagtttctgcgcgctcttgtacagaatggtgtgggtcatgtagtgcttgattctgctcaagaactagaacttcttgactatgttgcagcgggcgaaggcaagattcaagatgttttgattcgtgttaagccaggcattgaagctcatactcatgaattcatcgcgacgagtcatgaagatcagaaatttggattcagtctggcgtcaggttccgctttcgaagcggctaaagctgctaataacgctgagaatcttaatctggtagggctacattgtcatgtcggatcccaggtttttgatgctgagggtttcaaacttgcggctgaacgcgttttgggtttgtatagtcaaattcatagtgagcttggtgtagctctaccagaacttgatcttggtgggggatatggtattgcgtatactgcggctgaagaaccgcttaatgtagctgaagttgcgtccgatttgcttacggctgtcggtaaaatggcagcagaattgggtatcgatgcgccaactgttcttgtggaaccgggacgggctattgcgggtccatctacagttactatttacgaggtcggaacgactaaagatgtgcatgtcgatgatgataagactcgtcgctatattgctgtcgacggtggaatgtccgataacattcgtcctgcgctctatggatctgagtatgatgcacgcgtcgtatctcgttttgctgagggtgacccggtttcaactcgcatcgtgggatctcattgtgaatctggagacattctgatcaatgacgaaatttatccgtctgatattacgagtggtgattttcttgctttggctgcgactggcgcatattgttacgcaatgtctagtcgttataacgcttttactcgtccagctgttgtatccgtgcgggcgggatcatctcgccttatgcttagacgtgaaaccttggacgacattctttcccttgaggcgtag

SEQ ID NO: 17 - DNA, 1516 bp, Artificial Sequence
  promoter cg0007_39-codon-optimized lysA coding sequence-sod terminator
tgccgtttctcgcgttgtgtgtggtactacgtggggacctaagcgtgtattatggaaacgtctgtatcggataagtagcgaggagtgttcgttaaaaatggctaccgtagaaaacttcaatgaactacccgcgcatgtctggcctcgtaatgcagttcgccaggaagatggcgttgttacggtagcgggagtgccgcttcctgatcttgcagaagagtacggtacacctctctttgttgttgacgaagatgatttccgttctcgatgtcgtgatatggctaccgcatttgggggaccaggcaatgtgcattacgcttccaaagcgttccttactaaaactattgcacgctgggtagacgaagagggactagcgttggatatcgcgtctattaatgaactgggaattgcgttggcggctggattcccggcttcacgaatcacggcacatggtaataataaaggtgtcgagtttctgcgcgctcttgtacagaatggtgtgggtcatgtagtgcttgattctgctcaagaactagaacttcttgactatgttgcagcgggcgaaggcaagattcaagatgttttgattcgtgttaagccaggcattgaagctcatactcatgaattcatcgcgacgagtcatgaagatcagaaatttggattcagtctggcgtcaggttccgctttcgaagcggctaaagctgctaataacgctgagaatcttaatctggtagggctacattgtcatgtcggatcccaggtttttgatgctgagggtttcaaacttgcggctgaacgcgttttgggtttgtatagtcaaattcatagtgagcttggtgtagctctaccagaacttgatcttggtgggggatatggtattgcgtatactgcggctgaagaaccgcttaatgtagctgaagttgcgtccgatttgcttacggctgtcggtaaaatggcagcagaattgggtatcgatgcgccaactgttcttgtggaaccgggacgggctattgcgggtccatctacagttactatttacgaggtcggaacgactaaagatgtgcatgtcgatgatgataagactcgtcgctatattgctgtcgacggtggaatgtccgataacattcgtcctgcgctctatggatctgagtatgatgcacgcgtcgtatctcgttttgctgagggtgacccggtttcaactcgcatcgtgggatctcattgtgaatctggagacattctgatcaatgacgaaatttatccgtctgatattacgagtggtgattttcttgctttggctgcgactggcgcatattgttacgcaatgtctagtcgttataacgcttttactcgtccagctgttgtatccgtgcgggcgggatcatctcgccttatgcttagacgtgaaaccttggacgacattctttcccttgaggcgtagtaggcatttttagtacgtgcaataaccactctggtttttccagggtggttttttgatgccctttttggagtcttcaactgg

SEQ ID NO: 18 - protein, 445 aa, C. glutamicum NRRL B-11474
MATVENFNELPAHVWPRNAVRQEDGVVTVAGVPLPDLAEEYGTPLFVVDEDDFRSRCRDMATAFGGPGNVHYASKAFLTKTIARWVDEEGLALDIASINELGIALAAGFPASRITAHGNNKGVEFLRALVQNGVGHVVLDSAQELELLDYVAAGEGKIQDVLIRVKPGIEAHTHEFIATSHEDQKFGFSLASGSAFEAAKAANNAENLNLVGLHCHVGSQVFDAEGFKLAAERVLGLYSQIHSELGVALPELDLGGGYGIAYTAAEEPLNVAEVASDLLTAVGKMAAELGIDAPTVLVEPGRAIAGPSTVTIYEVGTTKDVHVDDDKTRRYIAVDGGMSDNIRPALYGSEYDARVVSRFAEGDPVSTRIVGSHCESGDILINDEIYPSDITSGDFLALAATGAYCYAMSSRYNAFTRPAVVSVRAGSSRLMLRRETLDDILSLEA

SEQ ID NO: 19 - DNA, 4508 bp, C. glutamicum NRRL B-11474
cttctccgactacatgcgtcctgcagttcgtcttgcagctctcatggagaccgacgcttactacgtctggacccacgactccatcggtctgggcgaagatggcccaacccaccagcctgttgaaaccttggctgcactgcgcgccatcccaggtctgtccgtcctgcgtcctgcagatgcgaacgagaccgcccaggcttgggctgcagcacttgagtacaaggaaggccctaagggtcttgcactaacccgccagaacattcctgttctggaaggcaccaaggagaaggctgctgaaggcgttcgccgcggtggctacgtcctggttgagggttccaaggaaaccccagatgtgatcctcatgggctccggctccgaggttcagcttgcagttaacgctgcgaaggctctggaagctgagggcgttgcagctcgcgttgtttccgttccttgcatggattggttccaggagcaggacgcagagtacatcgagtccgttctgcctgcagctgtgaccgctcgtgtgtctgttgaagctggcatcgcaatgccttggtaccgcttcttgggcacccagggccgtgctgtctcccttgagcacttcggtgcttctgcggattaccagaccctgtttgagaagttcggcatcaccaccgatgcagtcgtggcagcggccaaggactccattaacggttaattgccctgctgtttttagcttcaacccggggcaatatgattctccggaattttattgccccgggttgttgttgttaatcggtacaaagggtcttaagcacatcccttacttgcctgctctccttgagcacagttcaagaacaattcttttaaggaaaatttagtttcatgtctcacattgatgatcttgcacagctcggcacttccacttggctcgacgacctctcccgcgagcgcattacttccggcaatctcagccaggttattgaggaaaagtctgtagtcggtgtcaccaccaacccagctattttcgcagcagcaatgtccaagggcgattcctacgacgctcagatcgcagagctcaaggccgctggcgcatctgttgaccaggctgtttacgccatgagcatcgacgatgttcgcaatgcttgtgatctgttcaccggcatcttcgagtcctccaacggctacgacggccgcgtgtccatcgaggttgacccacgtatctctgctgaccgcgacgcaaccctggctcaggccaaggagctgtgggcaaaggttgatcgtccaaacgtcatgatcaagatccctgcaaccccaggttctttgccagcaatcaccgacgctttggctgagggcatcagcgttaacgtcaccctgatcttctccgttgctcgctaccgcgaagtcatcgctgcgttcatcgagggcatcaagcaggctgctgcaaacggccacgacgtatccaagattcactctgtggcttccttcttcgtctcccgcgtcgacgttgagatcgacaagcgcctcgaggcaatcggatccgatgaggctttggctctgcgcggcaaggcaggcgttgccaacgctcagcgcgcttacgctgtgtacatggagcttttcgacgccgccgagctgcctgaaggtgccaacactcagcgccctctgtgggcatccaccggcgtgaagaaccctgcgtacgctgcaactctttacgtttccgagctggctggtccaaacaccgtcaacaccatgccagaaggcaccatcgacgcggttctggagcagggcaacctgcacggcgacaccctgtccaactccgcggcagaagctgacgctgtgttctcccagcttgaggctctgggcgttgacttggcagatgtcttccaggtcctggagaccgagggtgtggacaagttcgttgcttcttggagcgaactgcttgagtccatggaagctcgcctgaagtagaatcggcacgctgcatcagtaacggcgacataaaatcgaatcagttcgatcttgtgtggccgttacacatctttcattaaagaaaggatcgtgacgctaccatcgtgagcacaaacacgacccccaccagctggacaaacccactgcgcgacccgcaggataaacgactcccccgcatcgctggcccttccggcatggtgatcttcggtgtcactggcgacttggctcgaaagaagctgcttcccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaatttcgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatatcggcttgggtggacgtgctggttactacgacggcatcggtgcagcccgcgacgtcatccagaaccacctgatccagctcttggctctggttgccatggaagaaccaatttctttcgtgccagcgcagctgcaggcagaaaagatcaaggtgctctctgcgacaaagccatgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttggatgaatccagcctcttccccaccaacgaggaagtggaactgagctggaagattctggatccaattcttgaagcatgggacgccgatggagaaccagaggattacccagcaggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaagcctcgcgcgagcacccatctcgcgtgatcattttggtggttggcgataaaactgcagaaaacaaagttgacgcagaagtccgtatcggtggcgacgctggtgcttccgagatgatcatcatgcatctcaacggacctgtcgctgacaagctccagcatgtcgtcacaccactgttgcttcctgacacccccatcgttgcttggtggccaggtgaatcaccaaagaatccttcccaggatccaattggacgcatcgcacaacgacgcatcactgatgctttgtacgaccgtgatgacgcactagaagatcgtgttgagaactatcacccccctgcgtacgctgcaactctttacgtttccgagctggctggtccaaacaccgtcaacaccatgccagaaggcaccatcgacgcggttctggagcagggcaacctgcacggcgacaccctgtccaactccgcggcagaagctgacgctgtgttctcccagcttgaggctctgggcgttgacttggcagatgtcttccaggtcctggagaccgagggtgtggacaagttcgttgcttcttggagcgaactgcttgagtccatggaagctcgcctgaagtagaatcggcacgctgcatcagtaacggcgacataaaatcgaatcagttcgatcttgtgtggccgttacacatctttcattaaagaaaggatcgtgacgctaccattgccgtttctcgcgttgtgtgtgg

SEQ ID NO: 20 - DNA, 97 bp, Artificial Sequence
  Promoter cg0007_39
tgccgtttctcgcgttgtgtgtggtactacgtggggacctaagcgtgtattatggaaacgtctgtatcggataagtagcgaggagtgttcgttaaaa

SEQ ID NO: 21 - DNA, 1643 bp, Artificial Sequence
  Promoter cg0007_39 insertion and zwf coding region
tgccgtttctcgcgttgtgtgtggtactacgtggggacctaagcgtgtattatggaaacgtctgtatcggataagtagcgaggagtgttcgttaaaagtgagcacaaacacgacccccaccagctggacaaacccactgcgcgacccgcaggataaacgactcccccgcatcgctggcccttccggcatggtgatcttcggtgtcactggcgacttggctcgaaagaagctgcttcccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaatttcgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatatcggcttgggtggacgtgctggttactacgacggcatcggtgcagcccgcgacgtcatccagaaccacctgatccagctcttggctctggttgccatggaagaaccaatttctttcgtgccagcgcagctgcaggcagaaaagatcaaggtgctctctgcgacaaagccatgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttggatgaatccagcctcttccccaccaacgaggaagtggaactgagctggaagattctggatccaattcttgaagcatgggacgccgatggagaaccagaggattacccagcaggtacgtggggtccaaagagcgctgatgaaatgctttcccgcaacggtcacacctggcgcaggccataat

SEQ ID NO: 22 - DNA, 5082 bp, C. glutamicum NRRL B-11474
ccctgcgtacgctgcaactctttacgtttccgagctggctggtccaaacaccgtcaacaccatgccagaaggcaccatcgacgcggttctggagcagggcaacctgcacggcgacaccctgtccaactccgcggcagaagctgacgctgtgttctcccagcttgaggctctgggcgttgacttggcagatgtcttccaggtcctggagaccgagggtgtggacaagttcgttgcttcttggagcgaactgcttgagtccatggaagctcgcctgaagtagaatcggcacgctgcatcagtaacggcgacataaaatcgaatcagttcgatcttgtgtggccgttacacatctttcattaaagaaaggatcgtgacgctaccattgccgtttctcgcgttgtgtgtggtactacgtggggacctaagcgtgtattatggaaacgtctgtatcggataagtagcgaggagtgttcgttaaaagtgagcacaaacacgacccccaccagctggacaaacccactgcgcgacccgcaggataaacgactcccccgcatcgctggcccttccggcatggtgatcttcggtgtcactggcgacttggctcgaaagaagctgcttcccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaatttcgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatatcggcttgggtggacgtgctggttactacgacggcatcggtgcagcccgcgacgtcatccagaaccacctgatccagctcttggctctggttgccatggaagaaccaatttctttcgtgccagcgcagctgcaggcagaaaagatcaaggtgctctctgcgacaaagccatgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttggatgaatccagcctcttccccaccaacgaggaagtggaactgagctggaagattctggatccaattcttgaagcatgggacgccgatggagaaccagaggattacccagcaggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaagcctcgcgcgagcacccatctcgcgtgatcattttggtggttggcgataaaactgcagaaaacaaagttgacgcagaagtccgtatcggtggcgacgctggtgcttccgagatgatcatcatgcatctcaacggacctgtcgctgacaagctccagcatgtcgtcacaccactgttgcttcctgacacccccatcgttgcttggtggccaggtgaatcaccaaagaatccttcccaggatccaattggacgcatcgcacaacgacgcatcactgatgctttgtacgaccgtgatgacgcactagaagatcgtgttgagaactatcacccaggtgataccgacatgacgtgggcgcgccttacccagtggcggggacttgttgcctcctcattggatcacccaccacacagcgaaatcacttccgtgaggctgaccggcgcaagcggcagtacctcggtggatttggctgcaggctggttggcgcgcaggctgaacgtgcctgtgatccgcgaggtgacagatgctcccaccgtgtcaaccgatgagtttggtactccactgctggctatccagcgcctggagatcgttcgcaccaccggctcgatcatcatcaccatccatgacgctcatacccttcaggtagagatgccggaatccggcaatgccccatcgctggtggctattggtcgtcgaagtgagtccgactgcttgtctgaggagcttcgccacatggatccagatttgggctaccagcacgcactatccggcttgtccagcgtcaagctggaaaccgtctaaggagaaatacaacactatggttgatgtagtacgcgcacgcgatactgaagatttggttgcacaggctgcctccaaattcattgaggttgttgaagcagcaactgccaataatagcaccgcacaggtagtgctcaccggtggtggcgccggcatcaagttgctggaaaagctcagcgttgatgcggctgaccttgcctgggatcgcattcatgtgttcttcggcgatgagcgcaatgtccctgtcagtgattctgagtccaatgagggccaggctcgtgaggcactgttgtccaaggtttctatccctgaagccaacattcacggatatggtctcggcgacgtagatcttgcagaggcagcccgcgcttacgaagctgtgttggatgaattcgcaccaaacggctttgatcttcacctgctcggcatgggtggcgaaggccatatcaactccctgttccctcacaccgatgcagtcaaggaatcctccgcaaaggtcatcgcggtgtttgattcccctaagcctccttcagagcgtgcaactctaacccttcctgcggttcactccgcaaagcgggtgtggttgctggtttctggtgcggagaaggctgaggcagctgcggcgatcgtcaacggtgagcctgctgttgagtggcctgctgctggagctaccggatctgaggaaacggtattgttcttggctgatgatgctgcaggaaatctctaagtagcgccagctctaacaagaagctctaacaagaagcttcaacaagaagctctaacgaaaagcactaacaaactaatccgggtgcgaaccttcatctgaatcgatggaggttcgcacccggatttttggctcagcgttagattctcaccctaaaaccgcgcacttaaaaaggttagctttctatacgtgcaccgcctctttgattggttcatgcgaggcgatccgaaagtcttcgctgtacagcggcgaggtgtttccgaccgctagttgtgctgcgagttcgccataagctggagagagcttgaaggcgtgtccggatcctccggtgagcacaatgacattgtccaccgcgtcgataattggagatttgtctgcagtgtaggtgtcatagtggacgctgaaacggttgggctctgggttgacccctgggaagagttcgtgggttttccgaccaaattcagagactgcgtcccggtcgaggcggagatcttcatcttctacatgagcgcttaatggaacgccccactcatccaatccggcaattttgatgctgtacccatcgacgcatggtgctccaaatacgtggaagccatcacggtcacggatgaagcatggcagattttccggctggaagtccactgggttgttgggcaggaaccaagtgagcactaggcgtcgcacttcaagcagtggcgcgatggagggcacgagctcgcttgtccagctgccggtggtgacgataacgcggtccacgattgtggtttcttcgcctgcttggatgactacgtgatcgccgttgtcttcgatgctggtgattttttggtgatcgcgtacttgggcaccatttgccttggctgtttcgattgcactgaacactgctagttcggggcgcagggctccaccttgaagatcaacaattccagcttcatcatcgcggaagtctagacctgggtaacgcttgcgcatctgcgcggcggtaagtcgttcatgtggcagctcataacgttccactgattccaccaggcgttggaagggtgcttcgtcttccttgccggtgcttaagacaccgaagttgtggaagagttcgcgtccggaaatctcgctcagtgatgaccatagtgctcgtgcgcgtttgagcaacggaacgtaggtgctgccttcgtggtaggccatgcgaaacagtcgggactcccctgtgaatgcgccgtagccatgggagatgccgaattgttcaaagccgatggcctctacacctgggatgttacttaagtgccacagtgccatggagccggttgatccaaggccgattaccgcaattttcattgttgtttcttctctcaagccagtgcgggtgattcc

SEQ ID NO: 23 - DNA, 951 bp, C. humireducens
atgaagatcgaattgcctgacaccgatacccgcaacattgcagcctccctgttggaagcccgcgaacacaactcccagacgactggtcgtgtactcaccctgatcgtagtggccgcattggatgatgacgtcgaccatatcatctccgtcacccgtgacgcgagccatgagcacccagctcgcgttctcgtgctccttacaggccgtgaagacgcggactctcgcctcgatgctgaagttcgcgttggaggtcacgccggcgcgtcagaaatggtaattatgcgcatcaccggagaaatggtgaaccacttggcggccgtcgtcaccccacttctgctgcctgacactcctgtagtggcgtggtggcctaccactgccccggaaaacccatcaggacaccctctgggacagctcgcccagcgacgtatcacaaacgcagatcatccggattccgaatctgtactccgtcttcgtcgagcgacttacgctccgggtgattctgacatggtgtggtccaagatcactgcctggcgtggtatcgtcgcttcttctctcgatcgccatccacatgaagacgtggaggcggccgtgctcactggacctgctgatgaaccacaaatcgatatcgcagcgggttggctggcagatcgcctgggtgtcccggtgcgccgcgaggtctgtgagcccggcaacgatgcccgcctgttcccgattcgccgtcttaagtttgagcgcgcttcaggtcctgttcgtgtagaggtactcgaccatcgcactgtgttggtggcccttccaggccgaccagaatcgctcgtggccatgaacgaacgctccgacgctgattgcctcagcgaggaattgcgccacctggaccccgatctggcatacgccaacgccttgcgcggactcacccgcgtgaagggcatcaatggccacccagcgtcgtcctga

SEQ ID NO: 24 - DNA, 1048 bp, Artificial Sequence
  promoter cg0007_39-C. humireducense opcA coding sequence
tgccgtttctcgcgttgtgtgtggtactacgtggggacctaagcgtgtattatggaaacgtctgtatcggataagtagcgaggagtgttcgttaaaaatgaagatcgaattgcctgacaccgatacccgcaacattgcagcctccctgttggaagcccgcgaacacaactcccagacgactggtcgtgtactcaccctgatcgtagtggccgcattggatgatgacgtcgaccatatcatctccgtcacccgtgacgcgagccatgagcacccagctcgcgttctcgtgctccttacaggccgtgaagacgcggactctcgcctcgatgctgaagttcgcgttggaggtcacgccggcgcgtcagaaatggtaattatgcgcatcaccggagaaatggtgaaccacttggcggccgtcgtcaccccacttctgctgcctgacactcctgtagtggcgtggtggcctaccactgccccggaaaacccatcaggacaccctctgggacagctcgcccagcgacgtatcacaaacgcagatcatccggattccgaatctgtactccgtcttcgtcgagcgacttacgctccgggtgattctgacatggtgtggtccaagatcactgcctggcgtggtatcgtcgcttcttctctcgatcgccatccacatgaagacgtggaggcggccgtgctcactggacctgctgatgaaccacaaatcgatatcgcagcgggttggctggcagatcgcctgggtgtcccggtgcgccgcgaggtctgtgagcccggcaacgatgcccgcctgttcccgattcgccgtcttaagtttgagcgcgcttcaggtcctgttcgtgtagaggtactcgaccatcgcactgtgttggtggcccttccaggccgaccagaatcgctcgtggccatgaacgaacgctccgacgctgattgcctcagcgaggaattgcgccacctggaccccgatctggcatacgccaacgccttgcgcggactcacccgcgtgaagggcatcaatggccacccagcgtcgtcctga

SEQ ID NO: 25 - protein, 316 aa, C. humireducens
MKIELPDTDTRNIAASLLEAREHNSQTTGRVLTLIVVAALDDDVDHIISVTRDASHEHPARVLVLLTGREDADSRLDAEVRVGGHAGASEMVIMRITGEMVNHLAAVVTPLLLPDTPVVAWWPTTAPENPSGHPLGQLAQRRITNADHPDSESVLRLRRATYAPGDSDMVWSKITAWRGIVASSLDRHPHEDVEAAVLTGPADEPQIDIAAGWLADRLGVPVRREVCEPGNDARLFPIRRLKFERASGPVRVEVLDHRTVLVALPGRPESLVAMNERSDADCLSEELRHLDPDLAYANALRGLTRVKGINGHPASS

SEQ ID NO: 26 - DNA, 4100 bp, C. glutamicum NRRL B-11474
acacaaagaagcaccaaccgatccggcgcttgcgcgtttgctccctgattttcaacacgagggcgatgaggaatacgacggcgataattctttcctccgttcacttcatgaaggcgatatcactcgcgcgaagctggagaatctgcgtgtgatcaatgatgcgctgggccccgacggcaatgttgcggttaccgcctctgaggaggaagcacacgcatggttggccgcgctcaatgatatccgcctgtacgttgcctccggtgatgcacgcggcggagaagccgccgaggaagaccgcgaaaacctcgtgcagtggctggcctacaatcaagagtccttgctggaagcgatgatgaattaatgcttatcgacgtcccgggcttccttttaggccacgtcacgaagggggatacgggttgctcagtggtcattgcacctaacggtgcatttgcgggcgtcgatgtccgtgggggaggcccaggcaccagggaaaccgaccttctagaaccacacaattctgtgcagcaagcacatgccgtggtgttgtgtggcggttcggcgttcgggttggctgctgccgatggagtgatgacagccctagaaaaccgcggtattggtttccctgtccgtcccgaagggcctatcgtgccaatcgttccaggcgctgtgatttttgatttgttggtgggtgaccccaaaaataggcccaccgcagctgatggggaacaggctgtagaaaacgctttcgctggtacacacaacggttcgggcagtgtcggtgcaggaaccggcgctacggcaggtcggctgcgtggcggttttggccaaagctcgcgccgggtcggaaagtacaccatcgcggcaggtgtggtggcgaatcctgttggggaagtcgtggacctaaagactggggctttgtttggtaggcccgaagtgatgggggtgggcgtcgataagctaaaaagcgcggcagagacgctgaacacgaccatcggcgtcgtggcaactgacgcgccggtgacaaaagcccaggcgaagcgcttggcgctggtggcccatgacggattggcgagggccgtgcggccgtcgcattcgccgatggacggtgacacattttttgctatgtcatcgggtgatggtagtggcgttaccccggttgagctgggggaattgtcggctcatgctgcagattgcgtacaggacgctattatcgacgccatacttaccgcgagtccgggactcgggctcaaaagcttcagggaacttttaccatgagttacaacagcccgtataacaacacgaatttcagcaccactggcgcgttccaacctgctggtgggccggtgaagccgtggaataagcctgatggcagcctgaatcagcagctgaaaaataaatcccgtgtgcgcacaggtcttaccatcgccatcggttatgttgtggtgatttgggcggtgcatttggcatccatcgtgattgcgctgctcactggcttcaacctgaccaactttggtattcatccgctggataccagtgcactgtggggtattttcgcctcaccgttgctgcatggaagcttcagtcacctcattggaaataccgttccaggctttatattcagtttcctcatcggtatgagtggcaagcgcgtgttctgggaagtcacgattatcgcaggtctcatcggcggtcttggtacatggattttcggtggaatcggcaccaaccacatcggtgcgtccggcctgatttatggctggcttggctacctgatcgtgcgtggaattttcaataaggatattaaacagttcctgcttggcatagttttggcgttcatttactccggtttgttctggggtctgctacctactcaaatcggtgtgtcctggcagggccaccttttcggtgcacttggtggaatcggtgcgggtgcttttatcgcctcggatgacccggcagcgttgaaagcgaagaagcagcagaagaaactagaaaagcaacaacgccaaagaggcttgtagttttcacctagcgactacacttaattgacatgacaagtgagaattccgaatcccaggacatttggctaaccgatgagcaacaagatgtgtggctcgatgtgtggacaatgcgaatcggcctgcctgctcgcttggatgctcaactgaaagaagctgcgggtgtcagccactttgagtacttcaccatggcgcagatttctatggccccggaacatcgggtgcgcatgagtgagcttgctgagctgtccgatatgacgctatcgcatctatctagagtggttactcgcctagaaaaggctggctgggtgaagcgtgttcccgatcccgatgatggtcgcgccaccgttgctgtgctcacggactctgggtgggagaaagttaaagcaacagcccctggtcatgtgaaggaagtgcgtcgtttggtgtttgacgatctcactccagaagaactcaaggtaatgggcactgcaatgaagaagattgtgaaccgactcgatatgtccaacaggctgccgcgggtgtagttagaagttttaagaaatgtagacaacggcgttgtcaccacctgtgcaggtgacatactcgccgttgtcttcgcagttcatggtgtagtttccgccagtgacagggctggtggctgtcaccctgccggatagttcattggtgtctaaatagtggtttacaaaagcatctcggacggcttgggcaaaacctgctgaggtggatgctgacccggtgtacacattgttcaaattgccggcatcggtctgggtggcagcagcatcattggctggtgatgctcccgcaggaagtgatggttgcgcagggcggtttcggggagtttccgttgcactgcttgggggctgatcctctgtcgcgctcggtgactcaggggattccgaaatagtttgctccacagattcagaggtgcttggacttgccacagggctcgaggaattatttggtgcaacgaggctgttgaacaacaagacaaacgcaatcacgatgatggccaccaagatcaagatcaacccaatgagactgcctttgggtgaacgtttcgcgggcgccacagctgtaggagagccaatagtctccgaagttgctcgcaccgcagcgaatcgtctggtttcttgatcaggtgaatcagccattgccatattgtgacacatcttggacggataaaaagggaagcaacgcgaggtagcttatgatggcaaccgtgactgatttcagtggatctatgattgaacgccccgtgccaggtgctgatgcgccgattggaatttttgattctggagttggcggattaaccgtagctcgcacaatcatcgatcaattgccacatgaatcagttatttatatcggtgatactgccaatggcccttatggtccgttgcctatcgctaaggtccgtgagcacgccatccgcattgccgatgagttggtggaacgcggatgcaagatgattgtcattgcctgtaacactgcgtccgctgcgtttctccgagatgcccgtgaacgatacagtgtgccagtcgtggaagttattcttcccgcagtaaggcgtgcggtggcatccacccgcaatggcaaagtgggcgtgatcggcacagtgggaaccattaactccggtgcgtaccaggatcttttctctgcaagcccctccattgaggtcaacgcagtggcatgcccacggtttgtggatttcgtggaacgcggaattaccagcggcaggcagatcctcaacattgcgcaggattatttagagcctctgcaagcagaaggggtggacactcttgtgcttggatgcacccactatccactgctctccggtgtcattcagttggcaatgggggaccacgtaagtttggtctctagcgcggaagaaactgcgaaagacgtgctgagaatcttgagccagcaagatcttttagccgatccggacatgcatcctgagccaagttatagctttgaatcaacaggcgatccggaaatctttgcacaattaagccgccgattccttggaccaattgtttccctagtgagacaaaacgagggataaccccaggtgtgtgt

SEQ ID NO: 27 - DNA, 595 bp, Artificial Sequence
  Promoter cg0007_39-cg2766
tgccgtttctcgcgttgtgtgtggtactacgtggggacctaagcgtgtattatggaaacgtctgtatcggataagtagcgaggagtgttcgttaaaaatgacaagtgagaattccgaatcccaggacatttggctaaccgatgagcaacaagatgtgtggctcgatgtgtggacaatgcgaatcggcctgcctgctcgcttggatgctcaactgaaagaagctgcgggtgtcagccactttgagtacttcaccatggcgcagatttctatggccccggaacatcgggtgcgcatgagtgagcttgctgagctgtccgatatgacgctatcgcatctatctagagtggttactcgcctagaaaaggctggctgggtgaagcgtgttcccgatcccgatgatggtcgcgccaccgttgctgtgctcacggactctgggtgggagaaagttaaagcaacagcccctggtcatgtgaaggaagtgcgtcgtttggtgtttgacgatctcactccagaagaactcaaggtaatgggcactgcaatgaagaagattgtgaaccgactcgatatgtccaacaggctgccgcgggtgtagtta

SEQ ID NO: 28 - DNA, 4283 bp, C. glutamicum NRRL B-11474
cgcatgatgtgctgatgcagatcatccaccgcgtcatcgcttttagccatctctagggcaagatctggttcaggatcaatgaggatctgacgaatatgatctgtcatgtcagctgcgaggcgggccatctccttgaaatagccacgcatgtcctccgggatcaccggatcggggtaacggcgacgcacggaattagctacgtgcattgccagagcacccatacgggtaatttcctcgacgatgtagatggaggagacaacctggcgaagatcacgggctacaggagtttcaagcgccaaaagttcgacggcttgagactcgcatccagtacgaagctcttcaagcttttctgcattgcctaaagcgctttctgccgatgccaaagaagtgcgcaggagggcatcagtcgccttatccatgcactccttagttagatcacacaaaatgattagatcgtgtgcgaattcatccaattgctgtctatatgcagtacgcatggcaacactataaggcgataatggtatttctgcagacctaaaacacccctttacggttgaatcacctaataatgggggatagccaactattggcgggggtaagtattaaattaactaccctccggagttttccatttctgcgcctttaggaacagtggcatcctcaggatcgtccaaccagccatctggaagtgccacctttgcaggagcgccctgtcgacctcgtgcaccttccgcgtcctctgccttggcggtggagtcagcccatggtgcaaggagatcctcaagctccacataggtggaaaccttggccagattggagcggaattcgccgccaacagggaaaccgcgcaggtaccaacccatgtgcttacgcagatcgcgcagccccttggtttcgccatcatgctgcatgaggagttctgcgtggcgcaggatgatttgggtaacttcgccgaaggtaggctcctctgggatttcttctccacgaacagcagcagacagctcagcaaagagccaaggcctgcccaggcaaccacgcccaaccacgacgccatcgcagccagtttgctccatcatgcgcgttgcatcggatgccgcgaaaatatcgccattgcccaaaactgggatgccggtatctgccaaatgctccttcaggcgcgcgatctcgttccaatcagcctcaccggaatagcgctgcgccgcagtgcgggcgtgaagcgctacggacttggcgcctgcgtcgacagcaatgcgtccagcatccaagtgagtatggtgctcatcatcaataccaacgcggaacttcaccgtcaccggaatgtccgtgccttccgtagccttcacagccgcggaaacgatgttttcaaacaaacggcgcttgtaaggaatcgcagaaccgccaccacggcgcgtgacctttggaaccgggcagccaaagttcatatcaatatgatccgccaagttttcatcaacgatcatcttcgccgcttcgtaggtgtacttcgggtcaaccgtgtacagctgcaagcttcggggattttcatccggcgcgaaggtggtcatgtgcatggttttctcattgcgctcaacaagagcacgcgcagtcaccatttcacagacgtacagccccgagattgttcccgtgcgttgcatttcctgttcgcggcacagcgtgcggaaagcaacgttggttacaccagccatgggggctagaaccacaggggaggcaaggtcaaaggggccgatttttagagtcacctaactattgtcccccgtgaatcaggttgggcaaaatatttgaagcaaattgtgagcagggcgcaactaggaaagtggtgtgctttcactttttgggggctggggttgggttaagcttcgcgggctctagggttggtctgagctttattcctgagctttattcctggtctttgggaggcttgcaaacagggggcatgcaaatttgggggtaatgctgggccttgaaatcccactatcacagatagtattcgggcatttcctgtcacgatggtttatccttgggacacaacatcaaagtggggtacatcatatgcttccggttgaagtgacctatctgaaaagattggtcgaaccttgaagcaatggtgtgaactgcgttaacgaattttgtcggacgttaaaatggtcgcattctgcttgctgaagtggcacacctatgtgttctgcttgggtatagcagtgcgggaaaaatttgaaaaagtccgattacctgaggaggtattcaatgtctgatcgcattgcttcagaaaagctgcgctccaagctcatgtccgccgacgaggcggcacagtttgttaaccacggtgacaaggttggtttctctggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaagtcacccgcatcactgaagacggacacctcatcccttcttcctccgtgggtaacaacgttgagtggctcaacgctgcagagaaggtcatcctcgaggttaactcttggcagtccgagaacctcgagggcatgcacgacatctggtctgtaccagcactgccaaaccgcatcgctgtgccaatcaacaaggccggcgaccgcatcggtaagacctacatcgaattcgacaccgacaaggttgtcgcaattgttgaaaccaacaccccagaccgcaacgcaccatttaagcctgtcgatgacatctctaagaagatcgccggtaacttcctcgacttcctcgaaggtgaagttgcagctggccgtctttcctacgatggttacgtcatgcagtccggcgtgggcaacgtgcctaatgctgtgatggcaggcctgctggagtccaagtttgagaacatccaggcttacaccgaggttatccaggacggcatggttgaccttatcgacgccggcaagatgaccgttgcttctgcaacctccttctccttgtctcctgagtacgcagagaagatgaacaacgacgcaaagcgttaccgtgagtccatcatcctgcgcccacagcagatctctaaccacccagaggtgatccgtcgcgtcggcctgatcgccaccaacggtcttatcgaagctgacatctatggcaacgttaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggcctggctccacgtgagcgcgttgccaagatgatcggcctggcacaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataagttttttcttggtttagaaaccgccgcctcgacaacatttcgaggcggcggtttcttttatcacctgggttttgagcgttaaattagaccaggtcaggctagtgtttggtagctaattgagggcgattttaataaggccggtgccatgtactaatatggtctgagttgggcctatagctcagttggtagagctacggacttttaatccgcaggtcttgggttcgagtcccaatgggcccacatcttaagtacccctgttttggagaatgctccgagccaggggtacttttcttttcctcacacacagtagctgctgagaaaaatgaagaccttttgttaggttgggagtatgaccaacccatacgaggccttcataccgctcaagcatcgtacggggattgaacccgagcacaccttttgggaatgggaaaacaaaagggttcacattgcaaggagacgtcgagaagcgcccgtccgcgttatcgtggtgcatgggctaggcacccatagtg

SEQ ID NO: 29 - DNA, 1606 bp, Artificial Sequence
  promoter cg0007_39-actA
tgccgtttctcgcgttgtgtgtggtactacgtggggacctaagcgtgtattatggaaacgtctgtatcggataagtagcgaggagtgttcgttaaaaatgtctgatcgcattgcttcagaaaagctgcgctccaagctcatgtccgccgacgaggcggcacagtttgttaaccacggtgacaaggttggtttctctggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaagtcacccgcatcactgaagacggacacctcatcccttcttcctccgtgggtaacaacgttgagtggctcaacgctgcagagaaggtcatcctcgaggttaactcttggcagtccgagaacctcgagggcatgcacgacatctggtctgtaccagcactgccaaaccgcatcgctgtgccaatcaacaaggccggcgaccgcatcggtaagacctacatcgaattcgacaccgacaaggttgtcgcaattgttgaaaccaacaccccagaccgcaacgcaccatttaagcctgtcgatgacatctctaagaagatcgccggtaacttcctcgacttcctcgaaggtgaagttgcagctggccgtctttcctacgatggttacgtcatgcagtccggcgtgggcaacgtgcctaatgctgtgatggcaggcctgctggagtccaagtttgagaacatccaggcttacaccgaggttatccaggacggcatggttgaccttatcgacgccggcaagatgaccgttgcttctgcaacctccttctccttgtctcctgagtacgcagagaagatgaacaacgacgcaaagcgttaccgtgagtccatcatcctgcgcccacagcagatctctaaccacccagaggtgatccgtcgcgtcggcctgatcgccaccaacggtcttatcgaagctgacatctatggcaacgttaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggcctggctccacgtgagcgcgttgccaagatgatcggcctggcacaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataa

SEQ ID NO: 30 - DNA, 35 bp, Artificial Sequence
  primer FarI 3p InfusF
atttcacaccgcatatgcggcatccttcagaccgc

SEQ ID NO: 31 - DNA, 43 bp, Artificial Sequence
  primer  FarI 3p InfusR AvrII
tgggatccgtcgacctgcagcctaggatctctggaccggaatc

SEQ ID NO: 32 - DNA, 34 bp, Artificial Sequence
  primer  FarI 5p InfusF
gtccagagatcctaggatcccgccgagaaaaccg

SEQ ID NO: 33 - DNA, 34 bp, Artificial Sequence
  primer  FarI 5p Infus R
aacagctcactctagaagtggaggtcatctgcac

SEQ ID NO: 34 - DNA, 71 bp, Artificial Sequence
  primer Pcg0007_39-zwf_P1
tttatgcttccggctcgtatgttgtgtggaattgtgagcggataacaattcttctccgactacatgcgtcc

SEQ ID NO: 35 - DNA, 95 bp, Artificial Sequence
  primer Pcg0007_39-zwf_P2
tatccgatacagacgtttccataatacacgcttaggtccccacgtagtaccacacacaacgcgagaaacggcaatggtagcgtcacgatcctttc

SEQ ID NO: 36 - DNA, 92 bp, Artificial Sequence
  primer Pcg0007_39-zwf_P3
tactacgtggggacctaagcgtgtattatggaaacgtctgtatcggataagtagcgaggagtgttcgttaaaagtgagcacaaacacgaccc

SEQ ID NO: 37 - DNA, 76 bp, Artificial Sequence
  primer Pcg0007_39-zwf_P4
ctgcaaggcgattaagttgggtaacgccagggttttcccagtcacgacgtgggtgatagttctcaacacgatcttc

SEQ ID NO: 38 - DNA, 76 bp, Artificial Sequence
  primer SNP_084_P1
tttatgcttccggctcgtatgttgtgtggaattgtgagcggataacaattcggtttcctgattgctcatcaaaatc

SEQ ID NO: 39 - DNA, 50 bp, Artificial Sequence
  primer SNP_084_P2
catgctgattaccactggtactcagagtgagcctatggctgcgctgtctc

SEQ ID NO: 40 - DNA, 50 bp, Artificial Sequence
  primer SNP_084_P3
gagacagcgcagccataggctcactctgagtaccagtggtaatcagcatg

SEQ ID NO: 41 - DNA, 73 bp, Artificial Sequence
  primer SNP_084_P4
ctgcaaggcgattaagttgggtaacgccagggttttcccagtcacgacgtaaaaactagaactgctcaaggcc

SEQ ID NO: 42 - DNA, 76 bp, Artificial Sequence
  primer Pcg1860_pyc_P1
tttatgcttccggctcgtatgttgtgtggaattgtgagcggataacaattcgcagtcatccggtggagaagacttc

SEQ ID NO: 43 - DNA, 91 bp, Artificial Sequence
  primer Pcg1860_pyc_P2
attttaaatacgcaagctactttatgtgtatgtgggacaatttgcaactatttgtgcaggtcaaagctaagtttcgggcaacactcccctg

SEQ ID NO: 44 - DNA, 94 bp, Artificial Sequence
  primer Pcg1860_pyc_P3
agttgcaaattgtcccacatacacataaagtagcttgcgtatttaaaattatgaacctaaggggtttagcagtgtcgactcacacatcttcaac

SEQ ID NO: 45 - DNA, 77 bp, Artificial Sequence
  primer Pcg1860_pyc_P4
ctgcaaggcgattaagttgggtaacgccagggttttcccagtcacgacgtcagagagatcaccagaataagccatag

SEQ ID NO: 46 - DNA, 77 bp, Artificial Sequence
  primer SNP_033_P1
tttatgcttccggctcgtatgttgtgtggaattgtgagcggataacaattaggtttcttacctacttatcaacggtg

SEQ ID NO: 47 - DNA, 40 bp, Artificial Sequence
  primer SNP_033_P2
gatcggcgcttcacgcttgagccgcaggagctgcgttttg

SEQ ID NO: 48 - DNA, 40 bp, Artificial Sequence
  primer SNP_033_P3
caaaacgcagctcctgcggctcaagcgtgaagcgccgatc

SEQ ID NO: 49 - DNA, 70 bp, Artificial Sequence
  primer SNP_033_P4
ctgcaaggcgattaagttgggtaacgccagggttttcccagtcacgacgtgctgcagacaggtagtcacc

SEQ ID NO: 50 - DNA, 72 bp, Artificial Sequence
  primer SNP_316_P1
tttatgcttccggctcgtatgttgtgtggaattgtgagcggataacaattcacctgcgacaacaccgagaac

SEQ ID NO: 51 - DNA, 40 bp, Artificial Sequence
  primer SNP_316_P2
ctgtggcagatcagcgtccgacaagaaatccgccgctgtg

SEQ ID NO: 52 - DNA, 40 bp, Artificial Sequence
  primer SNP_316_P3
cacagcggcggatttcttgtcggacgctgatctgccacag

SEQ ID NO: 53 - DNA, 70 bp, Artificial Sequence
  primer SNP_316_P4
ctgcaaggcgattaagttgggtaacgccagggttttcccagtcacgacgtcccaggcctgaaagttctgc

SEQ ID NO: 54 - DNA, 71 bp, Artificial Sequence
  primer SCS_aceE_P1
tttatgcttccggctcgtatgttgtgtggaattgtgagcggataacaattcatcgtctaaaaggcctgggc

SEQ ID NO: 55 - DNA, 44 bp, Artificial Sequence
  primer SCS_aceE_P2
caagttttgcttgatcggccaattccacacctcctgttggaatg

SEQ ID NO: 56 - DNA, 44 bp, Artificial Sequence
  primer SCS_aceE_P3
cattccaacaggaggtgtggaattggccgatcaagcaaaacttg

SEQ ID NO: 57 - DNA, 72 bp, Artificial Sequence
  primer SCS_aceE_P4
ctgcaaggcgattaagttgggtaacgccagggttttcccagtcacgacgttcgacaccctcgttggtggaag

SEQ ID NO: 58 - DNA, 76 bp, Artificial Sequence
  primer opcA_P1
tttatgcttccggctcgtatgttgtgtggaattgtgagcggataacaattccctgcgtacgctgcaactctttacg

SEQ ID NO: 59 - DNA, 74 bp, Artificial Sequence
  primer opcA_P2
atacacgcttaggtccccacgtagtaccacacacaacgcgagaaacggcatttttgcccctaaattatggcctg

SEQ ID NO: 60 - DNA, 79 bp, Artificial Sequence
  primer opcA_P3
gcggactcacccgcgtgaagggcatcaatggccacccagcgtcgtcctgaggagaaatacaacactatggttgatgtag

SEQ ID NO: 61 - DNA, 75 bp, Artificial Sequence
  primer opcA_P4
ctgcaaggcgattaagttgggtaacgccagggttttcccagtcacgacgtggaatcacccgcactggcttgagag

SEQ ID NO: 62 - DNA, 70 bp, Artificial Sequence
  primer pcg0007_39-cg2766_P1
tttatgcttccggctcgtatgttgtgtggaattgtgagcggataacaattacacacacctggggttatcc

SEQ ID NO: 63 - DNA, 98 bp, Artificial Sequence
  primer pcg0007_39-cg2766_P2
tactacgtggggacctaagcgtgtattatggaaacgtctgtatcggataagtagcgaggagtgttcgttaaaaatgacaagtgagaattccgaatccc

SEQ ID NO: 64 - DNA, 103 bp, Artificial Sequence
  primer pcg0007_39-cg2766_P3
tatccgatacagacgtttccataatacacgcttaggtccccacgtagtaccacacacaacgcgagaaacggcagtcaattaagtgtagtcgctaggtgaaaac

SEQ ID NO: 65 - DNA, 70 bp, Artificial Sequence
  primer pcg0007_39-cg2766_P4
ctgcaaggcgattaagttgggtaacgccagggttttcccagtcacgacgtacacaaagaagcaccaaccg

SEQ ID NO: 66 - DNA, 72 bp, Artificial Sequence
  primer 184f7primer pcg0007_39-actA_P1
tttatgcttccggctcgtatgttgtgtggaattgtgagcggataacaattcactatgggtgcctagcccatg

SEQ ID NO: 67 - DNA, 95 bp, Artificial Sequence
  primer 184f7primer pcg0007_39-actA_P2
tactacgtggggacctaagcgtgtattatggaaacgtctgtatcggataagtagcgaggagtgttcgttaaaaatgtctgatcgcattgcttcag

SEQ ID NO: 68 - DNA, 97 bp, Artificial Sequence
  primer 184f7primer pcg0007_39-actA_P3
tatccgatacagacgtttccataatacacgcttaggtccccacgtagtaccacacacaacgcgagaaacggcagatgttgtgtcccaaggataaacc

SEQ ID NO: 69 - DNA, 70 bp, Artificial Sequence
  primer 184f7primer pcg0007_39-actA_P4
ctgcaaggcgattaagttgggtaacgccagggttttcccagtcacgacgtcgcatgatgtgctgatgcag

SEQ ID NO: 70 - DNA, 48 bp, Artificial Sequence
  primer 184f7
cggtatttcacaccgcatactagtaccatccagaatcgtttctaagcg

SEQ ID NO: 71 - DNA, 42 bp, Artificial Sequence
  primer 184F8
gggatccgtcgacctgcatccacgaagccacgagataatctg

SEQ ID NO: 72 - DNA, 37 bp, Artificial Sequence
  primer 244-4Go F2 Inf SpeI
ggatcttggtactagtcgctgagacagttgagagaag

SEQ ID NO: 73 - DNA, 35 bp, Artificial Sequence
  primer 253-4Go R3
ggatcttggtactagtaggaattcgatgcggatcc

SEQ ID NO: 74 - DNA, 37 bp, Artificial Sequence
  primer 685
gaattcctgcagcccggggtaccacactgtttccttg

SEQ ID NO: 75 - DNA, 43 bp, Artificial Sequence
  primer 686
tcgagctcggtacccgggactagtccaagtgcgtggcgagttt

SEQ ID NO: 76 - DNA, 46 bp, Artificial Sequence
  primer 687
ccaagcttgcatgcctgcaggactagtaacgggcggtgaagggcaa

SEQ ID NO: 77 - DNA, 45 bp, Artificial Sequence
  primer 693
cttcgaaatgtttgacctgcaggacagcctttaaacgctcctttt

SEQ ID NO: 78 - protein, 718 aa, C. glutamicum
MNDSRNRGRKVTRKAGPPEAGQENHLDTPVFQAPDASSNQSAVKAETAGNDNRDAAQGAQGSQDSQGSQNAQGSQNRESGNNNRNRSNNNRRGGRGRRGSGNANEGANNNSGNQNRQGGNRGNRGGGRRNVVKSMQGADLTQRLPEPPKAPANGLRIYALGGISEIGRNMTVFEYNNRLLIVDCGVLFPSSGEPGVDLILPDFGPIEDHLHRVDALVVTHGHEDHIGAIPWLLKLRNDIPILASRFTLALIAAKCKEHRQRPKLIEVNEQSNEDRGPFNIRFWAVNHSIPDCLGLAIKTPAGLVIHTGDIKLDQTPPDGRPTDLPALSRFGDEGVDLMLCDSTNATTPGVSGSEADVAPTLKRLVGDAKQRVILASFASNVYRVQAAVDAAVASNRKVAFNGRSMIRNMEIAEKLGYLKAPRGTIISMDDASRMAPHKVMLITTGTQGEPMAALSRMARREHRQITVRDGDLIILSSSLVPGNEEAVFGVINMLAQIGATVVTGRDAKVHTSGHGYSGELLFLYNAARPKNAMPVHGEWRHLRANKELAISTGVNRDNVVLAQNGVVVDMVNGRAQVVGQIPVGNLYVDGVTMGDIDADILADRTSLGEGGLISITAVIDNRTGRLLERPTVQTSGFSEDAKSMMGEVTELSETTMNDLAAEGENDPYRMVQQLRRKLSRFVEQKWKRQPVIMPTVIPMTAETTHIGDDEVRASRESL

SEQ ID NO: 79 - protein, 709 aa, C. glutamicum
MNDSRNRGRKVTRKAGPPEAGQENHVETPVFQAPDASSNQSTVKAETAGNDNRDSSQGSQAQGSQAKESGDAKSGNNNRNRSSNNRRGGRGRRGSGNANEGSNNNGGNRGNRGNRGGGRRNVVKSMQGADLTQRLPEPPKAPSNGLRIYALGGISEIGRNMTVFEYNNRLLIVDCGVLFPSSGEPGVDLILPDFGPIEDHLHRVDALVVTHGHEDHIGAIPWLLKLRHDIPILASRFTLALIAAKCKEHRQRPKLIEVNEKSSEDRGPFNIRFWAVNHSIPDCLGLAIKTPAGLVIHTGDIKLDQTPPDGRPTDLPALSRFGDEGVDLMLCDSTNATTPGVSGSEADVAPTLKRLVGDAKQRVILASFASNVYRVQAAVDAAVAANRKVAFNGRSMIRNMEIAEKLGYLKAPRGTIISMDDASRMAPHKVMLITTGTQGEPMAALSRMARREHRQITVRDGDLIILSSSLVPGNEEAVFGVINMLAQIGATVVTGRDAKVHTSGHGYSGELLFLYNAARPKNAMPVHGEWRHLRANKELAISTGVNRDNVVLAQNGVVVDLVNGRAQVVGQIPVGNLYVDGVTMGDIDADILADRTSLGEGGLISITAVIDNRTGRLLERPTVQTSGFSEDAKSMMGEVTELAETTMNDLAAEGENDPYRMVQQMRRKLSRFVEQKWKRQPVIMPTVIPMTAETRHIDDDEVRASRESL

SEQ ID NO: 80 - protein, 683 aa, C. ulcerans
MTEPRNRSRKVTRKAGPPETTEAPAFQAPDTSVEVSAKDEAEKKTSGEGSSNQEASSKGNRGRSNSGRAGNGTRNGRSRRGGNPQNNKSNRGRRNVVKSMQGADLTERLPEPPKAPKNGLRIYALGGISEIGRNMTVFEYNNKMLLVDCGVLFPSSGEPGVDLILPDFGPIEDKLDKVEALVVTHGHEDHIGAIPWLLKLRPDLPIYASKFTLALIAAKCREHRQRPKLIEVNEKSDINRGPFNIRFWAVNHSIPDCLGLAIKTGAGLVIHTGDIKLDQTPTDGRPTDLPALSRFGDEGVDLMLCDSTNATTPGVSGSEADIAPTLKRLVGDAKQRVILASFASNVYRVQAAVDAAVASGRKVAFNGRSMIRNMEIAEKMGYLKAPRGTIVSMDDAAKMAPHKVMLITTGTQGEPMAALSRMARREHRQITVRDGDLIILSSSLVPGNEEAVFGVINMLAQIGATVVTSRDAKVHTSGHGYSGELLFLYNAARPVNAMPVHGEWRHLRANKELAISTGVERDRVVLAQNGVVVDLVDGRARVVGQIQIGNLYVDGVTMGDIDAGVLEERTSLGEGGLIAITAVIDNRTGRLLERPTVQAKGFSEDAVAMMPEVTELVENTMTDLAGEGENDPYRMVQQLRRRVSRFVEQKWRRKPMIMPTVIPTTQTQVEADEEEIRATRESL

SEQ ID NO: 81 - protein, 484 aa, C. glutamicum
MVWGMEHTSALTLIDSVLDPDSFISWDETPQYDNLNQGYAETLERARSKAKCDESVITGEGTVEGIPVAVILSDFSFLGGSLGTVASVRIMKAIHRATELKLPLLVSPASGGARMQEDNRAFVMMVSITAAVQRHREAHLPFLVYLRNPTMGGAMASWGSSGHLTFAEPGAQIGFLGPRVVELTTGRALPDGVQQAENLVKTGVIDGIVSPLQLRAAVAKTLKIIQPAEATDRFSPTTPGAALPVMEAIARSRDPHRPGIGEIMETLGADVVRLSGARAGALSSAVRVALARIGGRPVVLIGQDRRFTLGPQELRFARRGISLARELNLPIVSIIDTSGAELSQAAEELGIASSIARTLSKLIDAPLPTVSVIIGQGVGGGALAMLPADLVYAAENAWLSALPPEGASAILFRDTNHAAEIIERQGVQAHALLSQGLIDGIVAETEHFVEEILGTISNALSELDNNPERAGRDSRFTRFERLAQ

SEQ ID NO: 82 - protein, 480 aa, C. glutamicum MB001
MEHTSALTLIDSVLDPDSFISWNETPQYDNLNQGYAETLERARSKAKCDESVITGEGTVEGIPVAVILSDFSFLGGSLGTVASVRIMKAIHRATELKLPLLVSPASGGARMQEDNRAFVMMVSITAAVQRHREAHLPFLVYLRNPTMGGAMASWGSSGHLTFAEPGAQIGFLGPRVVELTTGHALPDGVQQAENLVKTGVIDGIVSPLQLRAAVAKTLKVIQPVEATDRFSPTTPGVALPVMEAIARSRDPQRPGIGEIMETLGADVVKLSGARAGALSPAVRVALARIGGRPVVLIGQDRRFTLGPQELRFARRGISLARELNLPIVSIIDTSGAELSQAAEELGIASSIARTLSKLIDAPLPTVSVIIGQGVGGGALAMLPADLVYAAENAWLSALPPEGASAILFRDTNHAAEIIERQGVQAHALLSQGLIDGIVAETEHFVEEILGTISNALSELDNNPERAGRDSRFTRFERLAQ

SEQ ID NO: 83 - protein, 480 aa, C. crudilactis
MAYTSALSLIDTVLDSDSFASWDTPPRYGNIDPKYAETLAKARSKSECNESIITGEGTVDGIAVAVILSDFSFLGGSLGTVASMRIMNAIHRATERKMPLLVSPASGGARMQEDNRAFVIMVSITAAVQRHREAHLPFLVYLRNPTMGGAMASWGSSGHITFAEPGAQIGFLGPRVVELSTGKPLPEGVQQAENLVARGVIDGIVSPMQLRAAVSKTVKVLQPAVLGAHFPTTAPGSGLPVMEAIARSRDPRRPGIGEIIETLGEDVVKLSGARRGTAMRAVRVGLARLAGRPVVLIGQDRHFALGPQDLSFARRGISLARELNLPIVSIIDTSGAELSQDAEELGIASSIARTLAKLIDAPLPTVSVILGQGVGGGALAMLPADKVYAAENAWLSALPPEGASAILFRSTDHAAEIMERQGVQAQTLLSQGLIDGIISEAVPFVEEVLGTISNALSELASNPERAGRESRFKRFERLAR

SEQ ID NO: 84 - protein, 414 aa, C. glutamicum
MAFGFFSRRKKKNKDKNPNENSAVPAHSEDSPQEVFEGNGRQVGDPIEQQVDRDARGRLTAADFLPDADLPQLNRSRANMLRRELEYRFSLQNAHINIDGNTAMIQRSDGGAAHVSLRTLAMNAAGLDNFDQLPELVESFVHGTLADATLNDLSTADLYKALRLRLLPTPGEGDDLVEHGLDRESQIRDDSILRTFTSDMSIALVLDTEHAIRIQPLKELEEFDDLSALERAADRNTWQELYDANVDASFVDAESDSEGSSFWAFESNSYYLGSAPLFLNDLLAKWAPDLDQSDGVIFAVPDRDLLIARPVTTGEDLMNGITAMVRIAMRFGLGNPTSISPRLHLLRDNQVTTFTDFRVVSPEMEAEWEDSAFDAPPAGAIGIEVRPDPYLMERLQQGGFGDFGDFGKPRDLDM

SEQ ID NO: 85 - protein, 383 aa, C. crudilactis
MAFGFFGRKKERKDRHSGDPERDSKSRLTSADLLPDTDLPQLNRSRANMLRQELEYRFALQDAHINIDGHSAMVQRADGGAAHVSLRTLAMNAAGLENFDQLPELVENFVHGTLADATLKDLSTADLYKALRLRLLPTPSENDELAEYGLTIESKIRDDSILRTFTSDMSIALVLDTEHAIRIQPLKELERFDDLGALERAADRNTWQELYDASVDVTFVDAESDSEGSSFWTFESNSYYLGSAPLFLGDLLAKWAPDLDQSDGVIFAVPDRDLLIARPISTGENLMNGITAMVRTAMRFGLGNPTSISPRLHMLHDNQVITFTDYRIVSPEMEAEWEENSFDAPPAGAIGIEVRPDPYLMERLQQGGFGDFGDFGKPRDLGM

SEQ ID NO: 86 - protein, 373 aa, C. efficiens YS-314
MEQKVERDGNGILTPADFLPDTDLPQLNRSRANMLRRELEYRFALKDAQITIDGASAMVHRPDGGAAHVSLRTLAMNAAGLDDLGQLPELVDNFVHGTLADATLSTLSTADLYKSLRLRLLPNPEQGDSILETSDGVERQIRETSVLRDFTSDTSIALVLDTEHAIRIQPLHELEEFDSLDALERAADRNTWQELLDADVDVTYFNNDEGGEGSDFWAFESSSYYLGSSPLFLTDLLHRWAPDLDQSRGVIFAVPDRDLLIARPVTTGENLMNGITSMVRIAMRFGLGNPTSISPRLHLLYDNQINTFTDYRIIEPTEDEEWAVPTADAPQPGSIGIEVRPDAYLMEMLQQDGFGDSFDDFGPRDLGPDDFRY

SEQ ID NO: 87 - protein, 316 aa, C. vitaeruminis DSM 20294
MIFELPDSSTRDIAKTLVRIRDTGGQVTTSRVLSLIVIARKEDDVEAVIRATNEASREHPSRVIVLIQGDPTVPSSRLDAEVRIGGDAGASELILINLDGEVANNLVHVVTPLLLPDTPIVTWWAYAAPVDPSSDPMGIIAQRRITDSLFDPPQDALYNRRNHYVPGDSDIAWARLTPWRGVLASTLDQPPHEMVQEVRVVGPQDSPSVDLAAGWLIDRLGVPVSRLVDEHAQACDADTIPVNRVELDRPSGTVVLEVLPDQDTLSVTFPGKEVALVAISRRSTSDCLAEELRHLDPDVAYQRALSGLAHVDYPQG

SEQ ID NO: 88 - protein, 317 aa, C. matruchotii ATCC 33806
MIFTLSDTDTHEIARTLVEIRDDGSQGTTSRVLTLIAVAKDTDNIRSVISASKDASREHPCRVIVLVTGDPDRNTRIDAEVRIGGDAGASEIIVIDLEGEVTRHLVHVVTPLLLPDTPIVVWWPYTAPVNPAEDPLGRIAQRRITDSQKDPLVDALYNRRNSYTDGDSDLAWARITPWRGVVASALDQPPFEPVIEAKVYGATNCPSVDLAAGWLAARLGVPVTRIAVDDPSVKKQASLNVTPVERVELVRPSGATVLEALDSQTIGVSVPNREPARVAVNLRTDADCLAEELRHLDPDHAYDEVLRGLNQVSYPVV

SEQ ID NO: 89 - protein, 353 aa, C. falsenii DSM 44353
MIIDLPNTSTHIISQKLSALREERGEVATGRVLTLIVVASSEDDLPRIIDATHEASREHPARVLILVNHRKSDEVHLDAEIRLGGDAGASEIIVMHNHGELVAHRASVVTPLLLPDTPIVAWWPTNGPRNPAADAIGMLATRRITDSLHDDDTDALFRRRMTYTRGDSDMAWSRITLWRGLLASSLDQPPYEPIDSVELTGPADDPSTDIAAGWLAEKLQVPVTRHNSGTDAIPRDDKGNKTISIEKAVLHRESATVEVTVVDSETVSMKVGDKESLVTLGRRPLADCLAEELRHLDPDYAFGHALRGLVRVNRPERTNRLPNYRGASIPDEAPDRWSASVADDHFAEQESRR

SEQ ID NO: 90 - protein, 310 aa, C. halotolerans YIM 70093
MIITLPDTDSREVAASLLEAREQNSRTTGRVLTLIVAANLNDNVDAIISAARGASHEHPARVLVFITGETSGKAQLDAEVRVGGDAGASEMVIMRVSGELAGHLESVVTPLLLPDTPIVTWWPTEAPENPSAHPLGRLSQRRITNAQRDGGEDILDRLRAAYTPGDSDMMWSRITAWRGIVASSLDQHPHGAVESATLVGPPEDPSVDIAAGWLADRLGVEIIREDCEPAEDPTLFPIRSLRFNRAPGPLKVDVIDHRTVQVTLPGRPESLVAMSVRSDSECLSEELRHLDPDTAYAQALRALNRVTHRS

SEQ ID NO: 91 - protein, 325 aa, C. pyruviciproducens ATCC BAA-1742
MHQLRNTSTKEIASKLEQIETSGMQPRTNHVLTLIVVINGKTNKDRLFHTISSSSKEHPSRVLVLEPLAEEHSLGFHASSVIDAELHVGGDAGASEIIWMQLRGEVTEHLDSVVTPLLLPDTPIVVWWPASAPVNPSHDPLGKLAMRRITDSFFDPAEDAIYRRRSNYTPGDSDLCWSRLTPWRGILASALDQPPFEPVRSAKVFGLAVSPTVDLAAGWLADVLGVPVYREVRGTADRPVDHNGNPATPVAEVILERDTGSVRLVVADSETIELTIGDGPATKIAQGRRPDVDCLSEEMRHLVPDQAYAGALDGLSKVTYLEADQ

SEQ ID NO: 92 - protein, 303 aa, C. casei UCMA 3821
MITPMPNTTTSKIARGLVEAQEHYTLTTSRVLTLIVVVNVDDEVDAVLDSVRDASHEHPSRVLVVITGSRDAEPLLNAELRVGGEAGASEIVVMHLFGPLADNAESVVTPLLLPDTPIVVWWPTAAPEGPSKTPIGKLGQRRITYAAAKGDLRRLSSGYAPGDTDMAWGEITQWRGIVASSLDRLPHEPVKRVEISGPSDSLAVDLAAAWMLDRLAVPVKRVVSDAPGEKFTIHSVRFFRETSDIIIDASEEGSVRVNVPGSPESMVALNTRSRAEILSEELRHLDADEAYAHTLRGLAQVDY

SEQ ID NO: 93 - protein, 350 aa, C. terpenotabidum Y-11
MTTPESTGVGSSGVGQLDGRMNLGRTTTHRITRALRSLREKRGEVTTGRVLTLIVSARSEEDLDSVVRGANEASREHPARVIVVIHDRSTDEVSLDAELIVGGQAGAAEIIFMRLHGELADHADSVVMPLLLPDTPVVVWWPFTSPRNPAGDSLGRLASRRITDSFSDAAAGKGAKAIFRRRIGYTPGDSDLVWSRITPWRGLLSSAVEQHAGDTVTGVDITGPAEDPAVDIAAGWLADRFSVPVTRYAGTSPAVPIDDEGLATAPVERVVLHFTGGDLVMEVLDHRSVRILDGETDNIVTLHRRTLGECLAEELRHLEPDAAFGDALHGLPRVRVAREPAPAPVEESHR

SEQ ID NO: 94 - protein, 319 aa, C. glutamicum NRRL B-11474
MIFELPDTTTQQISKTLTRLRESGTQVTTGRVLTLIVVTDSESDVAAVTESTNEASREHPSRVIILVVGDKTAENKVDAEVRIGGDAGASEMIIMHLNGPVADKLQHVVTPLLLPDTPIVAWWPGESPKNPSQDPIGRIAQRRITDALYDRDDALEDRVENYHPGDTDMTWARLTQWRGLVASSLDHPPHSEITSVRLTGASGSTSVDLAAGWLARRLNVPVIREVTDAPTVSTDEFGTPLLAIQRLEIVRTTGSIIITIHDAHTLQVEMPESGNAPSLVAIGRRSESDCLSEELRHMDPDLGYQHALSGLSSVKLETV

SEQ ID NO: 95 - DNA, 87 bp, Artificial Sequence
  promoter 3121
gtggctaaaacttttggaaacttaagttacctttaatcggaaacttattgaattcgggtgaggcaactgcaactctggacttaaagc

SEQ ID NO: 96 - DNA, 98 bp, Artificial Sequence
  promoter 0755
aataaatttataccacacagtctattgcaatagaccaagctgttcagtagggtgcatgggagaagaatttcctaataaaaactcttaaggacctccaa

SEQ ID NO: 97 - DNA, 86 bp, Artificial Sequence
  promoter 3381
cgccggataaatgaattgattattttaggctcccagggattaagtctagggtggaatgcagaaatatttcctacggaaggtccgtt

SEQ ID NO: 98 - DNA, 4093 bp, C. glutamicum
gtgatggcatcgcgcacctcaaactgtgcacccgcttctaagccaagcgctccaagatcaaggcgaacagtagcctcacgagcactacgtggatccaagttgaccacaatcaacacggtatttccggtcaaagcatcaaccttggagtaggcgatgatctgatcattgtctgcttcgtggaagtggatgttgcgtagctgctgcaaggcagggttcgcgcgacggatctggttgagcagagcgatgtaatcctcgagggaatcgccacgctccagagcgccctcgaaatcgcggggacgcagctcgtacttctcagaatccaagtactcttccgaaccaggcttgacggcctcgtgctcaaagagctcatatccggaatatacgccccacacaggagacatcgtggcggccaatgcggcgcggatagcgaacattgcgcgtccaccatgctgcagagacgcatgcaaaatgtcgggagtgttcacaaacaggttcggacgagaaatatccgccatgcgggcgatctcagtagcgaactcggtgagctcctccttggtgaccttccacgtgaagtaggtgtaagactgggagaaaccaatcttggccaagccatacagacgtgccgggcgagtaaacgcctccgctaggaaaatgacctcagggtttgatttatggatggcagaaataagccactgccagaaattagcgggcttagtgtgcgggttgtccacgcggaatgtggtcacacccaaatccacccagaacttcaccacacgatagacctcttcatagatcttcggagcagcattatcaaagttgatgggataaatatcctggtacttcttcggtgggttttctgcatacgcaatggtgccatcagccaacaccgtgaaaaactcgcggtgttcctgcgcccatggatgatcaggggcagcctgcagagctagatcgagtgcaacttccaaattgagttcccgtgcgcgagccaacaacgcctggaaatcttcaatggtgcccaaccgcggatgcgttgcatcatgcccaccatctttagaaccaatagcccacggcgaacccacatcatgaggttccggggtcagcgtattattgcggcccttgcggttgacctcgccaatcggatggatcggcgggaagtaaacagtatcgaagcccatcttcgcgacacgctccaacgcctgagcagtggtagcgaaagtgccatgaacgggggtgccggactcatcccaaccacctgtggaacgagggaaaagctcataccaagagttgaccaaagcggcacgacgctcgacacgaacctgcaggttttcacccatcgtgaccaactcacgcaacgggttaagttctaaaagatgagtgacactcgcggtgagcgctggggcgagacgtgcgcgtacatcgccgccacgccgcagagaggaggcgacgtcgaaaagcgcagtcctatcctctttagacaaattctccgcagcccgttcaaacagctgagccccgtgctcaaagtcgttatacaactcatctgaaccctggcctgcctcaattttagtggtaatcgcatgacgccacgtagacataggatccgaccacgcatccacacgaaatgtccaattaccagggacatcaggaacgaaaaatgcattcgactgatcctgattatcaggagtggggcgcatatgaatctgaataggctcagccgccactgaagaatcctccggaccggacacattcaacgtcgcggcaattgcatcatggccttcacgccacacaacagccgagacaggaacaatctcacccactactgctttcgctggatttccatccaaaatccggggacgaacatcatcgataccgagacggccagtcacagttttctcctcaacatgcagtgtgaagtgcgaacaaacgcacacaagcttaaaagttcgatcaagcagacacaccggcaattagccgggggtatgtctccattatgaaagcttaacggagctgtgtcacttccacacggaaccggagaaatcagtcaagatgggagatgtgactaactctataaaaaactctgcagctgcatcttttccctctgattcgagtgaactcaatgacgaattcaatgaagacctcctcatcgatttccaaggagtcttctttaagagggatggtcgaaacctcgtcggccccgtcgactggcaagtagaactagatgaacgctgggtaattatcggaccaaacggagccggcaaaaccacactcatccgcatggcatccgccgaagaattcccctccggaggaatggcacgcctcatgggagaacgcatcggcaaaactgacatgcgcgacctccgctccatgatcggcgtcagctcctccgcattaggcaaccgaatcccctccgaagaaaaagtctctgacctagtcatctccgcaggctacgcaatcctcggccgctggcgcgaagactacgacgaaatggacttcggacaagccaccgaaatcctcgaacaagtcggagccatgcacctagccgaccgcacctggggaaccctctccgaaggcgaacgcaaacgagtcctggtcgcacgcgcactcatgaccaacccggaactcctcatccttgacgaaccaaccgcaggaatggacctcggcggacgcgaagacctcgtcggctacctcggagaactcgccatggacccagacgcacctgccatcgtcatgatcacccaccacgtcgaagaaatccccgccggattcacccacgcaatgctcctcgacgaaggtgaaatcgtagcccaaggcttgatcaacaccgtcatgacaaacgagaacctatccaaagcattccaccagccaatccaagtagaccgcatcggggaacgctactttgcccgccgtgtgagaaccgccaggagtcatagggctcagtaggttttttggagttgtgggccggtgttggaggggtggttgtttaataggcgacttcacgtatgcggacttcatgtgcactgacttcttgcgctcaacttctcatgtgctgacttcactccggattgctggcatttggtagtgatgtcgtgcgatgagaagttgagtatgcgaagtcggtaccgagaagtcgacctttaggacatttttagaggagtccacgcagagactctgtctccgtaagttcgatttcacggttacgatttcgaactgtcgatatcccgggtgcggtttctcaattacaatttcgcacttccaatttctcaagagcgataccactaggaaaagtgccattttccgattgaaatcgccgttgagaaattgagcgcaaggtatcgagtttgcgaaatcggtaataagaaatcggtaatgagaaatcgtaaccgtgaaatcgaactttaaacccaaatgaccgaaaaagttcaaaccaacaagcatcaagtgacatattctcgtgcggactgcgcacggaatccatagactggtctgcagaagttttacgtgtgggtttgcttcggatggggctttaacaagcttcacagatgttggtttttcataggcaagctatccagaagcaggctttacagaaagtcagggtgtggcaatggtgatgcagggcattggtggtaggaagctggcggcgacggtgctgttggttcgggatgggatcatcaatgggcgtcctgatgtggaggtttacattcaggagcgtgtgtctactatggctaattttcctcgggcgacggtgtttccgggtgggggtgttgattctcgggattttgcggatggtcacggtaaggaagtgtggaggggacctagcgcggaggagtggggtgtgcgtttaggcgtggagcctcatgttgcgtatgcgttggtttttgctgcggttcgtgagttgtttgaagaggccggtacgttgcttgcggagcatacggatggttctggtttggtgaagaatgctggtcagtatcacggatatcgggagttgttggagactcatgagatgtcgctgacggatatgttgcagagtgagaatttggcgattcgtagtgatttgattgtgccttttgccaggtgggcgagccctgagggtaatagg

SEQ ID NO: 99 - DNA, 74 bp, Artificial Sequence
  primer Pcg0007_39-cg1383_P1
tttatgcttccggctcgtatgttgtgtggaattgtgagcggataacaattgtgatggcatcgcgcacctcaaac

SEQ ID NO: 100 - DNA, 96 bp, Artificial Sequence
  primer Pcg0007_39-cg1383_P2
tatccgatacagacgtttccataatacacgcttaggtccccacgtagtaccacacacaacgcgagaaacggcacttgactgatttctccggttccg

SEQ ID NO: 101 - DNA, 103 bp, Artificial Sequence
  primer Pcg0007_39-cg1383_P3
tactacgtggggacctaagcgtgtattatggaaacgtctgtatcggataagtagcgaggagtgttcgttaaaaatgggagatgtgactaactctataaaaaac

SEQ ID NO: 102 - DNA, 70 bp, Artificial Sequence
  primer Pcg0007_39-cg1383_P4
ctgcaaggcgattaagttgggtaacgccagggttttcccagtcacgacgtcctattaccctcagggctcg

SEQ ID NO: 103 - DNA, 991 bp, Artificial Sequence
  Pcg0007_39 promoter inserted in front of cg1383 start codon
tgccgtttctcgcgttgtgtgtggtactacgtggggacctaagcgtgtattatggaaacgtctgtatcggataagtagcgaggagtgttcgttaaaaatgggagatgtgactaactctataaaaaactctgcagctgcatcttttccctctgattcgagtgaactcaatgacgaattcaatgaagacctcctcatcgatttccaaggagtcttctttaagagggatggtcgaaacctcgtcggccccgtcgactggcaagtagaactagatgaacgctgggtaattatcggaccaaacggagccggcaaaaccacactcatccgcatggcatccgccgaagaattcccctccggaggaatggcacgcctcatgggagaacgcatcggcaaaactgacatgcgcgacctccgctccatgatcggcgtcagctcctccgcattaggcaaccgaatcccctccgaagaaaaagtctctgacctagtcatctccgcaggctacgcaatcctcggccgctggcgcgaagactacgacgaaatggacttcggacaagccaccgaaatcctcgaacaagtcggagccatgcacctagccgaccgcacctggggaaccctctccgaaggcgaacgcaaacgagtcctggtcgcacgcgcactcatgaccaacccggaactcctcatccttgacgaaccaaccgcaggaatggacctcggcggacgcgaagacctcgtcggctacctcggagaactcgccatggacccagacgcacctgccatcgtcatgatcacccaccacgtcgaagaaatccccgccggattcacccacgcaatgctcctcgacgaaggtgaaatcgtagcccaaggcttgatcaacaccgtcatgacaaacgagaacctatccaaagcattccaccagccaatccaagtagaccgcatcggggaacgctactttgcccgccgtgtgagaaccgccaggagtcatagggctcagtag

SEQ ID NO: 104 - DNA, 4044 bp, C. glutamicum
gacaggtggatatcggagtatcccatggagccggagttgatcttgttacgcatgattggatcagatgcgtatggcatgcgccagcggatagcgtcagcttctgcaagtacgccatcgcagtcaggggcggtcgatgcgccagtgaacaggtcgattgcgtagtcgttgcctgcaccgtgtgcttctttagcccggttagcgattgccgtaggcagtgcctttgggtagccagcgccggtgaagccagagaaaccaaccttgtcaccgtggttaacaaactgtgccgcctcgtcggcggacatgagcttggagcgcagcttttctgaagcaatgcgatcagacatttttaacgaacactcctcgctacttatccgatacagacgtttccataatacacgcttaggtccccacgtagtaccacacacaacgcgagaaacggcagatgttgtgtcccaaggataaaccatcgtgacaggaaatgcccgaatactatctgtgatagtgggatttcaaggcccagcattacccccaaatttgcatgccccctgtttgcaagcctcccaaagaccaggaataaagctcaggaataaagctcagaccaaccctagagcccgcgaagcttaacccaaccccagcccccaaaaagtgaaagcacaccactttcctagttgcgccctgctcacaatttgcttcaaatattttgcccaacctgattcacgggggacaatagttaggtgactctaaaaatcggcccctttgaccttgcctcccctgtggttctagcccccatggctggtgtaaccaacgttgctttccgcacgctgtgccgcgaacaggaaatgcaacgcacgggaacaatctcggggctgtacgtctgtgaaatggtgactgcgcgtgctcttgttgagcgcaatgagaaaaccatgcacatgaccaccttcgcgccggatgaaaatccccgaagcttgcagctgtacacggttgacccgaagtacacctacgaagcggcgaagatgatcgttgatgaaaacttggcggatcatattgatatgaactttggctgcccggttccaaaggtcacgcgccgtggtggcggttctgcgattccttacaagcgccgtttgtttgaaaacatcgtttccgcggctgtgaaggctacggaaggcacggacattccggtgacggtgaagttccgcgttggtattgatgatgagcaccatactcacttggatgctggacgcattgctgtcgacgcaggcgccaagtccgtagcgcttcacgcccgcactgcggcgcagcgctattccggtgaggctgattggaacgagatcgcgcgcctgaaggagcatttggcagataccggcatcccagttttgggcaatggcgatattttcgcggcatccgatgcaacgcgcatgatggagcaaactggctgcgatggcgtcgtggttgggcgtggttgcctgggcaggccttggctctttgctgagctgtctgctgctgttcgtggagaagaaatcccagaggagcctaccttcggcgaagttacccaaatcatcctgcgccacgcagaactcctcatgcagcatgatggcgaaaccaaggggctgcgcgatctgcgtaagcacatgggttggtacctgcgcggtttccctgttggcggcgaattccgctccaatctggccaaggtttccacctatgtggagcttgaggatctccttgcaccatgggctgactccaccgccaaggcagaggacgcggaaggtgcacgaggtcgacagggcgctcctgcaaaggtggcacttccagatggctggttggacgatcctgaggatgccactgttcctaaaggcgcagaaatggaaaactccggagggtagttaatttaatacttacccccgccaatagttggctatcccccattattaggtgattcaaccgtaaaggggtgttttaggtctgcagaaataccattatcgccttatagtgttgccatgcgtactgcatatagacagcaattggatgaattcgcacacgatctaatcattttgtgtgatctaactaaggagtgcatggataaggcgactgatgccctcctgcgcacttctttggcatcggcagaaagcgctttaggcaatgcagaaaagcttgaagagcttcgtactggatgcgagtctcaagccgtcgaacttttggcgcttgaaactcctgtagcccgtgatcttcgccaggttgtctcctccatctacatcgtcgaggaaattacccgtatgggtgctctggcaatgcacgtagctaattccgtgcgtcgccgttaccccgatccggtgatcccggaggacatgcgtggctatttcaaggagatggcccgcctcgcagctgacatgacagatcatattcgtcagatcctcattgatcctgaaccagatcttgccctagagatggctaaaagcgatgacgcggtggatgatctgcatcagcacatcatgcgtattctcacgctgcgtccttggcctcacgacaccaagagcgcggttgatctgacgctgctttcccgcttctacgagcgttacgccgatcacacggtaaacgtggccgcccgtatcatttacctgtccaccgggctgcacccggaggagtacatggaaaagcgcgagcaacaaagggccgatgccgacatggagaagcgctgggccgagctggagcggcagttccgcaccagcgagtaaaaagctgcttctcgacgctaaaagccactacccaccatctgaaattaatcaggtggtgggtagtggctttaattcttcgatttatccgaagcggccggagatgtaatcttcggtttccttctgatctgggttttcgaagatcttcttggtaggtccgatttcaaccagacgacctggcctaccggtcgcctccagggagtagaacgcggtctgatcggacacacgtgcagcctgctgcatgttgtgggtcacgatgacgatggtgaactcttccttcagctcgtggataaggtcctccacagccagggttgagattgggtcaagcgcggagcaaggctcgtccatgaggaggatttctggctcaaccgcgatcgcgcgagcgatgcacagacgctgctgctgaccaccggagaggccgccgcctggcttgtccagacgatccttaacctcttcccacaggtttgcgccacgaagagacttctcagcaacttccttgagcttcttcttgttcttctcgccggaaagcttcagacctgcaaccacgttgtcctcaatggacatggttgggaatgggttagccttctggaagaccatgccgatggtgttacggactgcaactgggtcgatcttggagccgtagatgttctcgccgtcgagaaggatctcacccttgacgtatgcacctggggtgacctcgtgcatacggttgatggagcgaagaactgtggacttgccacagccggatggtccgatgaatgcggtgacagagcgtgcgggaacttcgaggttcacgttctgcactgcgtggaaatcaccgtagtagatgttgacatcattgagcttgagcttcgacatcgctttagatactcctgttgtattttctgagattgttattgcttgacagagaacttggcggagatgattcgtgcgccaatgttcaggacagcgatgatgagcaccagggtgagggctgcgccccacagcttgtccagcgttgctggtgcggtgccggccttgtacatatcaagcatcatcagtggaagggaagcctgcggaccgctgaatgggttccagttgatggcctgggaggaaccgaccaagaccagaactggtgcggactcacccatgacacgtgcgactgcgagcatgacgccggtgacgataccggacagtgcggttgggagaacgatctttgcgatggtcttccactttggcacgcccagtgcgtaggacgcttcacgcagatcctgaggaacaacgcggagcatttcttcggtgtttcggatgatcactggaaccatcagaatcaccagtg

SEQ ID NO: 105 - DNA, 73 bp, Artificial Sequence
  primer Pcg0007_39-phoU_P1
tttatgcttccggctcgtatgttgtgtggaattgtgagcggataacaattgacaggtggatatcggagtatcc

SEQ ID NO: 106 - DNA, 98 bp, Artificial Sequence
  primer Pcg0007_39-phoU_P2
tatccgatacagacgtttccataatacacgcttaggtccccacgtagtaccacacacaacgcgagaaacggcaaatcacctaataatgggggatagcc

SEQ ID NO: 107 - DNA, 100 bp, Artificial Sequence
  primer Pcg0007_39-phoU_P3
tactacgtggggacctaagcgtgtattatggaaacgtctgtatcggataagtagcgaggagtgttcgttaaaaatgcgtactgcatatagacagcaattg

SEQ ID NO: 108 - DNA, 72 bp, Artificial Sequence
  primer Pcg0007_39-phoU_P4
ctgcaaggcgattaagttgggtaacgccagggttttcccagtcacgacgtcactggtgattctgatggttcc

SEQ ID NO: 109 - DNA, 832 bp, Artificial Sequence
  Pcg0007_39 promoter in front of phoU start codon
tgccgtttctcgcgttgtgtgtggtactacgtggggacctaagcgtgtattatggaaacgtctgtatcggataagtagcgaggagtgttcgttaaaaatgcgtactgcatatagacagcaattggatgaattcgcacacgatctaatcattttgtgtgatctaactaaggagtgcatggataaggcgactgatgccctcctgcgcacttctttggcatcggcagaaagcgctttaggcaatgcagaaaagcttgaagagcttcgtactggatgcgagtctcaagccgtcgaacttttggcgcttgaaactcctgtagcccgtgatcttcgccaggttgtctcctccatctacatcgtcgaggaaattacccgtatgggtgctctggcaatgcacgtagctaattccgtgcgtcgccgttaccccgatccggtgatcccggaggacatgcgtggctatttcaaggagatggcccgcctcgcagctgacatgacagatcatattcgtcagatcctcattgatcctgaaccagatcttgccctagagatggctaaaagcgatgacgcggtggatgatctgcatcagcacatcatgcgtattctcacgctgcgtccttggcctcacgacaccaagagcgcggttgatctgacgctgctttcccgcttctacgagcgttacgccgatcacacggtaaacgtggccgcccgtatcatttacctgtccaccgggctgcacccggaggagtacatggaaaagcgcgagcaacaaagggccgatgccgacatggagaagcgctgggccgagctggagcggcagttccgcaccagcgagtaa

SEQ ID NO: 110 - DNA, 4137 bp, C. glutamicum
cggcgtcggcaaatttgtagagggcttcttcggtgaaggttccggcaggccccaaataggccacaacaattggtgcgtcgctcatggttacacagcttaacccgccgaactaaggtgggtgtccatgtctgttgctcaattagcgaaccgcttggcccaactctcccccgccgagcatggttttgcatggttcgaccctgaaatcaccgctggccatggcgttggcccgttgcatggcatggtgattccagccaaggacctcaacgatgtcgtgggcatgcccaccgcgttcggaaatgcatctcggcgcaaggtagcaacagacaccgatccgttcatccaaaatctcatcgaccgcggcgcgatcatcgctggcaaaacccaaaccagcgagctcggcatgacggcgtattgcgagcccatcggcatggacgcacccagcaacccggttttacctggtcacacgcctggaggttcgtccggtggtgcggccgttgctgttgccaggtcgcttgtcgacgccgcccacgcctcagacggtggcggctcgatccgggttccagccgccgcgtgtggcctggtcgggtttaaacccgcccacgattcccgaggtggaaacccctccacgcaggggtttatcacccgcgacgtggccacccaagtgcgtttgcacgcacttcaaccacgcaccagacgcctgcgcatcggcgttctcgctgagcccatccatgctaattcgcttgtcgacgcccccttcctgagcatcctcgaatccaccgcccacctcctggagaaagctggccacgaaatagtgtccgtacccctcccttatgacgcttgggcttttgacgcttatacagaagttttcatgatgaaatccgctggactaaccaacctgggtagccccattacaagatggttgagtgaacaaggccgtagtctttctccttctgatagacaatcatgtgtcaaggcttttgattccgtggctgagactgtacacggagcatgggacatcgatgttttattaacccctaccttggcttatgcacctcccaagattgggtacttttcatccatgccacctgaagaagacttccttgcacaaaccaaatggacgccgtgggcaacgctgttcaacatgaccggtggtgcagccatcagcgtgcctgttgaaggtgtcggcattcatcttggtgggatacgcgtacgagatgaagacctcttaggattagcagcatttcttgaaggagctgtggcatgagtagttcagtaatgtcaccgcgtcgaataaaggcggaaatacttatcgttttagccatcacctttggcatgagcggtctgcgatcaatcctgcgcctcatcgacgatctcctcgcaccggttgcgctcaacgaccagcaagttgcactgaatgcctcaatggcaaactccgcatggctggatctcgccttgcaattgtgttccgccggtgtgctgttttcctggggagcgctagcgatctatctgcttggcgaacgattccgctggataaaacccaaagattggacatggggtgcaggccttgcagcgctcatcggcattcccgggctgatcttctatgcgagtgccgttcatctgggtctttccaaacaggtcgtgcccaccactttggaaacctggtgggaaatcccagtgctgcttatatggtcagccgccaacgcatttggtgaagagatcgtggtggtcatgtggttcttcaccagactgcgccaactgaagtggagcgtgccagcagtcattgtggcatcttcagttttgcgcggttcctaccacctctaccagggaatctctgcgggttttggcaacatcatcatgggagtagcgttcgcatatttctttcaccgcacgggcaaaatctggccactggtaatcgctcacttcttgattgatgcggtggcttttgtgggctactccgcaattggcgggaatttaagctggttagggctttaaactgaagttcctattttattgggattagaaaaacacgagagatttaaggcaaatccgctaaatacatctgtcccgcctaggcgctactctattaaccatggattcaccaggacagggcgaaatagcccgcgatagtcaaggacgcccgatactggaccggtatggtcggccggttcgggttcgtccgcaaccgcggcaaactcccccgactcctcgaacccctcccgtgaatgaaacaagggtctatcagccaaggcaaacgccaccgcgccagactcctccaaggcagacacctccacgacaaatgcctcctaggcaaaccccaccgaggcaggttcccccacaacagcagtaccagcagcctggtcagatcggacaccttcgcccgcagccgccagtgattacaaatggtggagggagacgtcgaaaagcaatatcttttaagccccgtggctgccttggctcgctcgcgggcctggttgctgtggtgctggtgctcgcttttgtggtcgcgctgtgggcggatgccaagctgaatcgcgtggatgcgacgcccgcgacgcaggtggcgaataccgcaggcacgaactggctgctggtgggttcggactcgcggcagggtttaagtgatgaggatattgagcggctgggtaccggcggagatatcggtgtgggtcgtacggacacgatcatggtgttgcatatgccgcgtactggcgagccgacgctgttgtcgattccgcgtgattcttatgtcaatgtccctggctggggcatggataaggcaaacgcggcgttcactgtgggtggcccgcagctgctcacgcagaccgtggaggaggcgactggtctgcgaattgatcactatgcggaaatcggcatgggtggtttggcgaacatggttgatgccgtgggcggcgtggaaatgtgtcctgctgagccgatgtatgatccgctggccaacctggatattcaagccggctgccaggaatttgatggggcgaccgcgctgggatacgtgcgcactcgtgccacatccctgggtgatctggaccgcgtggtgcgccagcgggagtttttctccgcgctgctgagtacggctacgtccccgggcacgttgctgaatccgttccgcaccttcccgatgatctccaacgcggtgggaacattcaccgtcggcgagggcgatcacgtgtggcacctggcccgattggcgctggcgatgcgcggaggaatcgtgacggagaccgtgccgattgcctcattcgcagattacgatgtgggaaatgttgcgatctgggacgaagctggagccgaagcactatttagctccatgcgctaaaaccccaggtaatcattcacaggaagtgtccagtcaactttttggttaattgattatcttacctagagacttggatacttccctccacggattgctaacgatccagttttatcgttggactcatgaaaaagatccacatgattccagcggccctcctcatcggttccctcgcacttgtcagctgcgcgccttcacaacccacccagaacgcgcagacggtttcttctgcaatgatggaaactcctacttatacttcatcatctaggtcggtggaatctaccgcggggacgatcgccgccaccgaggaagccgcgatgacgctgctggcgaatagttcgggggatccgggatctgattctgggggtgcgcgctatagcctcaattcgctcaaggtttccgagcaagccgcagcgaacgccgtattaaaggccgtgcttaacgacgcctcctaccaagagttcgcagactcctcctacttagaaatcacaggaacgccctccgccgacggaacatggggcatctcatttggcggaccttccgaatcggcctccgtggaattctccgacggcagcgtcagcttctcccccgtcgacatgacagtgcccgcaacacgattgcctcagatgggcgcattttatgaaaccctgaccgaagaacaattagacatgctggagactggcctcgcggtttccaccttggattcgagccaacaggaaatgcttctggacttggtctccaactcgatcggcctggcagatactgaaaccactgcgactgcaattagtgaaatccgagcaaaactt

SEQ ID NO: 111 - DNA, 70 bp, Artificial Sequence
  Pcg0007_39-cg3210-p1
tttatgcttccggctcgtatgttgtgtggaattgtgagcggataacaattcggcgtcggcaaatttgtag

SEQ ID NO: 112 - DNA, 103 bp, Artificial Sequence
  Pcg0007_39-cg3210-p2
tatccgatacagacgtttccataatacacgcttaggtccccacgtagtaccacacacaacgcgagaaacggcatcgtgtttttctaatcccaataaaatagga

SEQ ID NO: 113 - DNA, 93 bp, Artificial Sequence
  Pcg0007_39-cg3210-p3
tactacgtggggacctaagcgtgtattatggaaacgtctgtatcggataagtagcgaggagtgttcgttaaaaatggattcaccaggacaggg

SEQ ID NO: 114 - DNA, 80 bp, Artificial Sequence
  Pcg0007_39-cg3210-p4
ctgcaaggcgattaagttgggtaacgccagggttttcccagtcacgacgtagttttgctcggatttcactaattgcagtc

SEQ ID NO: 115 - DNA, 1342 bp, Artificial Sequence
  Pcg0007_39 in front of cg3210
tgccgtttctcgcgttgtgtgtggtactacgtggggacctaagcgtgtattatggaaacgtctgtatcggataagtagcgaggagtgttcgttaaaaatggattcaccaggacagggcgaaatagcccgcgatagtcaaggacgcccgatactggaccggtatggtcggccggttcgggttcgtccgcaaccgcggcaaactcccccgactcctcgaacccctcccgtgaatgaaacaagggtctatcagccaaggcaaacgccaccgcgccagactcctccaaggcagacacctccacgacaaatgcctcctaggcaaaccccaccgaggcaggttcccccacaacagcagtaccagcagcctggtcagatcggacaccttcgcccgcagccgccagtgattacaaatggtggagggagacgtcgaaaagcaatatcttttaagccccgtggctgccttggctcgctcgcgggcctggttgctgtggtgctggtgctcgcttttgtggtcgcgctgtgggcggatgccaagctgaatcgcgtggatgcgacgcccgcgacgcaggtggcgaataccgcaggcacgaactggctgctggtgggttcggactcgcggcagggtttaagtgatgaggatattgagcggctgggtaccggcggagatatcggtgtgggtcgtacggacacgatcatggtgttgcatatgccgcgtactggcgagccgacgctgttgtcgattccgcgtgattcttatgtcaatgtccctggctggggcatggataaggcaaacgcggcgttcactgtgggtggcccgcagctgctcacgcagaccgtggaggaggcgactggtctgcgaattgatcactatgcggaaatcggcatgggtggtttggcgaacatggttgatgccgtgggcggcgtggaaatgtgtcctgctgagccgatgtatgatccgctggccaacctggatattcaagccggctgccaggaatttgatggggcgaccgcgctgggatacgtgcgcactcgtgccacatccctgggtgatctggaccgcgtggtgcgccagcgggagtttttctccgcgctgctgagtacggctacgtccccgggcacgttgctgaatccgttccgcaccttcccgatgatctccaacgcggtgggaacattcaccgtcggcgagggcgatcacgtgtggcacctggcccgattggcgctggcgatgcgcggaggaatcgtgacggagaccgtgccgattgcctcattcgcagattacgatgtgggaaatgttgcgatctgggacgaagctggagccgaagcactatttagctccatgcgctaa

SEQ ID NO: 116 - DNA, 4233 bp, C. glutamicum
cttttgccctcggtggccaaacatccgctgagtcctaccttgtcattgacaagatcatcgatgcggcccgcaagtccggcgccgacgccatccaccctggctacggcttcctcgcagaaaacgctgacttcgcagaagcagtcatcaacgaaggcctgatctggattggaccttcacctgagtccatccgcgccctcggcgacaaggtcaccgctcgccacatcgcagataccgccaaggctccaatggctcctggcaccaaggaaccagtaaaagacgcagcagaagttgtggctttcgctgaagaattcggtctcccaatcgccatcaaggcagctttcggtggcggcggacgtggcatgaaggttgcctacaagatggaagaagtcgctgacctcttcgagtccgcaacccgtgaagcaaccgcagcgttcggccgcggcgagtgcttcgtggagcgctacctggacaaggcacgccacgttgaggctcaggtcatcgccgataagcacggcaacgttgttgtcgccggaacccgtgactgctccctgcagcgccgtttccagaagctcgtcgaagaagcaccagcaccattcctcaccgatgaccagcgcgagcgtctccactcctccgcgaaggctatctgtaaggaagctggctactacggtgcaggcaccgttgagtacctcgttggctccgacggactgatctccttcctcgaggtcaacacccgcctccaggtggaacacccagtcaccgaagagaccaccggcatcgacctggtccgcgaaatgttccgcatcgcagaaggccacgagctctccatcaaggaagatccagctccacgcggccacgcattcgagttccgcatcaacggcgaagacgctggctccaacttcatgcctgcaccaggcaagatcaccagctaccgcgagccacagggcccaggcgtccgcatggactccggtgtcgttgaaggttccgaaatctccggacagttcgactccatgctggcaaagctgatcgtttggggcgacacccgcgagcaggctctccagcgctcccgccgtgcacttgcagagtacgttgtcgagggcatgccaaccgttatcccattccaccagcacatcgtggaaaacccagcattcgtgggcaacgacgaaggcttcgagatctacaccaagtggatcgaagaggtttgggataacccaatcgcaccttacgttgacgcttccgagctcgacgaagatgaggacaagaccccagcacagaaggttattgtggagatcaacggccgtcgcgttgaggttgcactcccaggcgatctggcactcggtggcaccgctggtcctaagaagaaggccaagaagcgtcgcgcaggtggcgcaaaggctggcgtatccggcgatgcagtggcagctccaatgcagggcactgtcatcaaggtcaacgtcgaagaaggcgctgaagtcaacgaaggcgacaccgttgttgtcctcgaggctatgaagatggaaaaccctgtgaaggctcataagtccggaaccgtaaccggccttactgtcgctgcaggcgagggtgtcaacaagggcgttgttctcctcgagatcaagtaagttcttggattcctgttagtcaaaaaccgggtcgcatctgatgttcagatgtggcccggttttactatttgtgaatacactctgcccctgttgtacattcgacaacaagatttcggtaaaaaggtggtcaaaatcaggaaatcttgttgtcgaatgtacaccgcattgtgggctttcacggttcgctgcgttgattgttatttagccagcccgctccccacacttaaccggtgatcaacgagcccacatacccggcgcggtgctggaacagcccgagaaattcaagatcgtttggggttggctgctcgcccgctgatacttctcgttcaatggagtggatgactgcttcgcggattgcggttccatcggcgtagccttgccagttgatctcggtggtgtctttcatggtgtgctccccttgtggttttttattttctttaacgctaatcggctgacggctttcaatgcacaaggtaaaatttgtgtaattgtgtcgtgtggatttgaaggtttttgtaaatctagttaaatcatgaggtcataagcttttcgggaggtggtcaaaagatgtcgaagctttacgcgggggcaaggatcaatgcactgcgccgaacccaccaactgacccaatcagcattagccgacaagcttgatctctccacgagctatctcaaccagttggaaaatgatggccggccgcttactgccacggtgcttttgcaactgatgaaagtctttgatgttgaggccagttatttctcccctgaccggggcacagccacggctacccgactggcagaaaccttggctatgaatcaggggccgacgatgtcgatggatgatctgttagatttcgcggatcgtttccctcagttggcacagcatattatccagcctgctgaggtcgatcccgcacatagttctgcgcatgattttgttcgggattattttgccacccacaagaactacattgattctcttgatcgccttggtgaggagttggcaaccgccattggtcagccggggcttcgggttaccaggctcgcgcagttgcttgatgcggagtacaacatcacggtgcgtttccgggcgccagatattactggccggaggcactttgatccccaatcgcgtcagattctgctgcggcaagatctcagcgaggcgcagcagtgttttcagttggcggaggaattgacgtttcttgctcatgcagagctcttggataccctgaccacagatcaaccggatctcccttatgaggcagctatccgcctggctaaggtgggtctctcccaatacttcgcggctgctgttgtcatgccgtacacccgctttttggaattcgcccaggacaagcactatgacatcgagttgatctctgaggcgtttggagtgtctttcgagtccgcgtgccaccgcttgtctacgctgcagcgttcgggggcgtcaggggtgccgtttttctttgtgcgctcggaccgggcaggaaatatttccaagaggcaatctgcagctacgttccatttctcgcgaacagacggcacttgtcctttgtgggcgctgcatcgagcttttgaacgtcaaggaaacatcacccgccaggttgctcgcatgccggatggccggacctatttgtggctcgcacgcgcggtgaaaggtcgaactcatggtttcgggcatcctgctgcggaattcgccatcggcctgggctgcgatatcagcgaggcaccaggcttggtgtattcccaaggcctcaatttggatccagagtccgccgcagagatcggccctggttgtcggatctgtcctcgggagaactgtgtgcagcgtgcattcccaccatcgggtcaagaatctatccgcccagcccctgtccaactcctcaactaaaaaggcagcacttaaaaaacactatggaacccatggaactaaacggcggtcgtttctacgcccgcaagctcacctcagataatcggattgatgatgtcccggcgctctcactgatccttcccgatgcggcacagttcatcgcggacgccaccacagcctggcacgaggacaccgaatacacctgggctatctgcgagcagaccagtgttgaactactggctttggcacgccttgatcccaagcagcgcacactcgaggtcgtgcccgcaggtgatcccgccacccaattgcccaatgatccagtgctcgctccgaaaaccatcggcgatgctgtggcggagggccgcgacaccatcaccagatgggcacaaggctatttagaaatcagcctcactgattaggttctctccgaaatgggcaccacctccctttcttccactccgttgtaggcagacaatggtcggatcagcgcattgttttcaaattgctccacgatgtgtgccgtccaccctgacactcgggccagcacaaacagcggtgtgaaaaagtcgacggggaatcccagcatgtaataggcagggccggccgggaagtcgaggttgggtttaatgccagtgcgagcctccatgacttcctgcatcgactcatacatgtgcacccatttttgaccacggtgacgagcagcaagggagcgcatggatttctccatggaggggaccctgga

SEQ ID NO: 117 - DNA, 76 bp, Artificial Sequence
  Pcg0007_39-cg0800-p1
tttatgcttccggctcgtatgttgtgtggaattgtgagcggataacaatttccagggtcccctccatggagaaatc

SEQ ID NO: 118 - DNA, 92 bp, Artificial Sequence
  Pcg0007_39-cg0800-p2
tactacgtggggacctaagcgtgtattatggaaacgtctgtatcggataagtagcgaggagtgttcgttaaaaatgtcgaagctttacgcgg

SEQ ID NO: 119 - DNA, 93 bp, Artificial Sequence
  Pcg0007_39-cg0800-p3
tatccgatacagacgtttccataatacacgcttaggtccccacgtagtaccacacacaacgcgagaaacggcaaaaccacaaggggagcacac

SEQ ID NO: 120 - DNA, 72 bp, Artificial Sequence
  Pcg0007_39-cg0800-p4
ctgcaaggcgattaagttgggtaacgccagggttttcccagtcacgacgtcttttgccctcggtggccaaac

SEQ ID NO: 121 - DNA, 1423 bp, Artificial Sequence
  Pcg0007_39 in front of cg0800
tgccgtttctcgcgttgtgtgtggtactacgtggggacctaagcgtgtattatggaaacgtctgtatcggataagtagcgaggagtgttcgttaaaaatgtcgaagctttacgcgggggcaaggatcaatgcactgcgccgaacccaccaactgacccaatcagcattagccgacaagcttgatctctccacgagctatctcaaccagttggaaaatgatggccggccgcttactgccacggtgcttttgcaactgatgaaagtctttgatgttgaggccagttatttctcccctgaccggggcacagccacggctacccgactggcagaaaccttggctatgaatcaggggccgacgatgtcgatggatgatctgttagatttcgcggatcgtttccctcagttggcacagcatattatccagcctgctgaggtcgatcccgcacatagttctgcgcatgattttgttcgggattattttgccacccacaagaactacattgattctcttgatcgccttggtgaggagttggcaaccgccattggtcagccggggcttcgggttaccaggctcgcgcagttgcttgatgcggagtacaacatcacggtgcgtttccgggcgccagatattactggccggaggcactttgatccccaatcgcgtcagattctgctgcggcaagatctcagcgaggcgcagcagtgttttcagttggcggaggaattgacgtttcttgctcatgcagagctcttggataccctgaccacagatcaaccggatctcccttatgaggcagctatccgcctggctaaggtgggtctctcccaatacttcgcggctgctgttgtcatgccgtacacccgctttttggaattcgcccaggacaagcactatgacatcgagttgatctctgaggcgtttggagtgtctttcgagtccgcgtgccaccgcttgtctacgctgcagcgttcgggggcgtcaggggtgccgtttttctttgtgcgctcggaccgggcaggaaatatttccaagaggcaatctgcagctacgttccatttctcgcgaacagacggcacttgtcctttgtgggcgctgcatcgagcttttgaacgtcaaggaaacatcacccgccaggttgctcgcatgccggatggccggacctatttgtggctcgcacgcgcggtgaaaggtcgaactcatggtttcgggcatcctgctgcggaattcgccatcggcctgggctgcgatatcagcgaggcaccaggcttggtgtattcccaaggcctcaatttggatccagagtccgccgcagagatcggccctggttgtcggatctgtcctcgggagaactgtgtgcagcgtgcattcccaccatcgggtcaagaatctatccgcccagcccctgtccaactcctcaactaa

(19) United States
(12) Patent Application Publication
(10) Pub. No.: US 2024/0254526 A1
(43) Pub. Date: Aug. 1, 2024

(54) ENGINEERED STRAINS OF CORYNEBACTERIA

(71) Applicant: Archer Daniels Midland Company, Decatur, IL (US)

(72) Inventors: Shawn Manchester, Decatur, IL (US); Peter Enyeart, Decatur, IL (US)

(73) Assignee: Archer Daniels Midland Company, Decatur, IL (US)

(21) Appl. No.: 17/274,093

(22) PCT Filed: Sep. 6, 2019
(86) PCT No.: PCT/US19/49893

§ 371 Date: Mar. 5, 2021

Related U.S. Application Data
(60) Provisional application No. 62/728,384, filed on Sep. 7, 2018.

(51) Int. Cl. C12P 13/08; C12N 1/20; C12N 15/77; C12R 1/15

(52) U.S. Cl. CPC C12P 13/08; C12N 1/205; C12N 15/77; C12N 2800/101; C12R 2001/15

(57) ABSTRACT

This disclosure provides engineered strains of Corynebacteria for the cost-effective production of lysine, tools and methods used to produce the engineered strains, and methods of using the engineered strains to produce lysine.

### CROSS-REFERENCE TO RELATED APPLICATIONS

This application is a national stage entry of International Application No. PCT/US19/49893, filed Sep. 6, 2019, which itself claims priority to U.S. Provisional Patent Application No. 62/728,384, filed Sep. 7, 2018, the contents of each are incorporated herein by reference.

Each reference, patent, and published patent application cited in this disclosure is incorporated herein by reference in its entirety.

This application incorporates by reference a 195 kb text file created on Sep. 7, 2018 and named “BIO066US00sequencelisting.txt,” which is the sequence listing for this application.

### TECHNICAL FIELD

This disclosure relates generally to lysine production.

### BRIEF DESCRIPTION OF THE FIGURES

FIG. 1A-B is a multiple alignment of SEQ ID NO:3 with the amino acid sequences of three corynebacterial ribonuclease J (rnaJ) proteins identified by National Center for Biotechnology Information (NCBI) Reference Nos. WP_011014791.1 (SEQ ID NO:78), WP_096456687.1 (SEQ ID NO:79), and WP_023635875.1 (SEQ ID NO:80). A box identifies the amino acid in each sequence that alignments with amino acid 448 of SEQ ID NO:3. FIG. 1A, alignment with amino acids 1-560 of SEQ ID NO:3. FIG. 1B, alignment with amino acids 561-718 of SEQ ID NO:3.

FIG. 2 is a multiple alignment of SEQ ID NO:6 with the amino acid sequences of three corynebacterial accDA proteins identified by NCBI Reference Nos. WP_075348128 (SEQ ID NO:81), AGT04828.1 (SEQ ID NO:82), and WP_066564978.1 (SEQ ID NO:83). A box identifies the amino acid in each sequence that alignments with amino acid 310 of SEQ ID NO:6.

FIG. 3 is a multiple alignment of SEQ ID NO:9 with the amino acid sequences of three corynebacterial cg1144 proteins identified by NCBI Reference Nos. WP_011897001 (SEQ ID NO:84), WP_066565124 (SEQ ID NO:85), and EEW49979 (SEQ ID NO:86). A box identifies the amino acid in each sequence that alignments with amino acid 66 of SEQ ID NO:9.

### DETAILED DESCRIPTION

This disclosure provides engineered strains of Corynebacteria for the cost-effective production of lysine, tools and methods used to produce the engineered strains, and methods of using the engineered strains to produce lysine.

### Structural Alterations

This disclosure provides the following four structural alterations that can be engineered into Corynebacteria to improve lysine production: (a) insertion of a promoter in front of the cg1383 start codon, (b) replacement of the native phoU promoter; (c) replacement of the native cg3210 promoter; and (d) replacement of the native cg0800 promoter.

a. Insertion of a Promoter in Front of the cg1383 Start Codon

cg1383 is also known as NCg11179 and encodes an ATPase component of the ABC-type molybdenum transport system (e.g., WP_011014216.1). In some embodiments, a promoter is inserted in front of the cg1383 start codon. The inserted promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the inserted promoter is promoter Pcg0007_39 (SEQ ID NO:20); see US 2017/0159045 and WO 2017/00376.

b. Replacement of the Native phoU Promoter

phoU encodes a phosphate uptake regulator (e.g., BAB99964.1) and is also known as cgl2571 and NCgl2482. In some embodiments, the native phoU promoter is replaced with a phoU replacement promoter. The phoU replacement promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the phoU replacement promoter is promoter Pcg0007_39 (SEQ ID NO:20); see US 2017/0159045 and WO 2017/00376.

c. Replacement of the Native Cg3210 Promoter

cg3210 is also known as NCgl2802 and encodes a cell envelope-related transcriptional regulator (e.g., WP_011266029.1). In some embodiments, the native cg3210 promoter is replaced with a cg3210 replacement promoter. The cg3210 replacement promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the cg3210 replacement promoter is promoter Pcg0007_39 (SEQ ID NO:20); see US 2017/0159045 and WO 2017/00376.

d. Replacement of the Native Cg0800 Promoter

cg0800 is also known as NCg10668 or prpR and encodes a transcriptional regulatory of the MerR family (e.g., WP_011013825.1). In some embodiments, the native cg0800 promoter is replaced with a cg0800 replacement promoter. The cg0800 replacement promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the cg0800 replacement promoter is promoter Pcg0007_39 (SEQ ID NO:20); see US 2017/0159045 and WO 2017/00376.

### Additional Structural Alterations

In some embodiments, engineered strains of Corynebacteria comprising one or more of the structural alterations described above may also comprise one or more of the additional structural alterations (e)-(o) described below.

e. Altered Corynebacterial Ribonuclease J Proteins

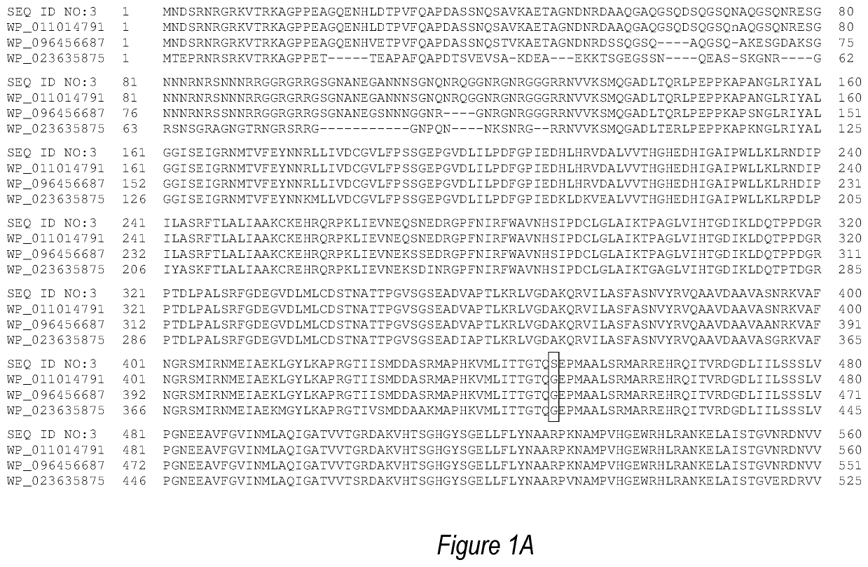

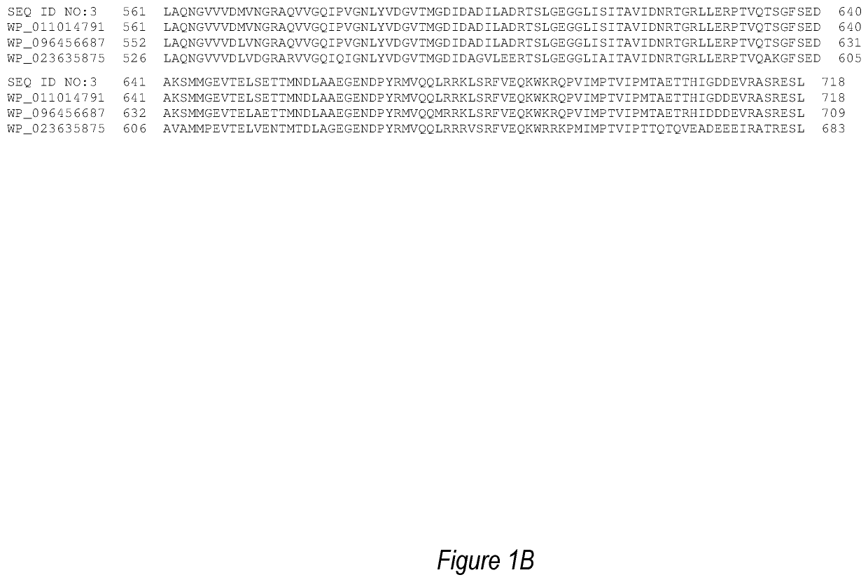

Engineered strains of Corynebacteria comprising any of the structural alterations described above can also comprise an altered corynebacterial ribonuclease (rnaJ) protein. Altered corynebacterial rnaJ proteins comprise a G448S substitution. One example of such a protein is shown in SEQ ID NO:3. “A corynebacterial rnaJ protein comprising a G448S substitution” as used herein means an rnaJ protein from a strain of Corynebacterium which, when compared with SEQ ID NO:3 using the NCBI's BLAST® alignment tool, has serine instead of glycine at the position that aligns with amino acid 448 of SEQ ID NO:3, as illustrated in FIG. 1A-B. Corynebacterial rnaJ proteins which can be altered to comprise the G448S substitution include, but are not limited to, proteins (regardless of how named) identified with the amino acid sequences provided by the NCBI Reference numbers in Table 1.

TABLE 1
NCBI Reference Numbers of Corynebacterial rnaJ Proteins

| Reference number | Species and/or Strain(s) |
|---|---|
| WP_011014791.1 | C. glutamicum 617, 1006, AJ1511, AS 1.299, |
| | ATCC 13032 (K051) and DSM 20300, ATCC |
| | 13869, ATCC 21831, C1, CP, MB001, TQ2223, |
| | XV, and ZL-; B. flavum ZL-1 |
| WP_003857476.1 | C. glutamicum AS 1.542, ATCC 13870, |
| | ATCC 14067, ATCC 21493, MT, S9114, SCgG1, |
| | SCgG2, SYPA 5-5, SYPS-062, SYPS-062-33a, |
| | T6-13, and Z188; B. flavum ATCC 15168 |
| WP_077312485.1 | C. glutamicum |
| WP_089158555.1 | C. glutamicum |
| WP_044030039.1 | C. glutamicum |
| WP_040967540.1 | C. glutamicum |
| WP_059289432.1 | C. glutamicum |
| WP_063967576.1 | C. glutamicum |
| WP_011897391.1 | C. glutamicum |
| WP_096456687.1 | C. glutamicum |
| WP_080794542.1 | Corynebacterium sp. Marseille-P2417 |
| WP_015651527.1 | C. callunae |
| WP_011075647.1 | C. efficiens |
| WP_095075511.1 | C. ulcerans |
| WP_014836513.1 | C. ulcerans |
| WP_087454146.1 | C. ulcerans |
| WP_013911733.1 | C. ulcerans |
| WP_014525921.1 | C. ulcerans |
| WP_023635875.1 | C. ulcerans |
| WP_014300762.1 | C. pseudotuberculosis |
| WP_013242122.1 | C. pseudotuberculosis |
| AJC14005.1 | C. pseudotuberculosis |
| WP_058832312.1 | C. pseudotuberculosis |
| WP_014523406.1 | C. pseudotuberculosis |
| WP_014558731.1 | C. pseudotuberculosis |
| OMH75460.1 | C. pseudotuberculosis |
| WP_014367221.1 | C. pseudotuberculosis |
| WP_092284332.1 | C. spheniscorum |
| WP_020976441.1 | C. argentoratense |
| WP_075726535.1 | C. aquilae |
| WP_027012113.1 | C. freiburgense |
| WP_034995241.1 | C. matruchotii |
| WP_005526573.1 | C. matruchotii |
| WP_018339963.1 | C. caspium |
| WP_018020369.1 | C. ciconiae |
| WP_023019341.1 | Corynebacterium sp. KPL1995 and KPL1989 |
| OUJ24662.1 | C. kefirresidentii |
| WP_018119731.1 | C. propinquum |
| ART21077.1 | C. striatum |
| ERS47112.1 | Corynebacterium sp. KPL1856 |
| WP_021352644.1 | C. pseudodiphtheriticum |
| WP_027017879.1 | C. pseudodiphtheriticum |
| WP_075691844.1 | C. sphenisci |
| WP_046202207.1 | C. kroppenstedtii |
| WP_012731758.1 | C. kroppenstedtii |
| WP_064833244.1 | Corynebacterium sp. |
| | EPI-003-04-2554_SCH2473622 |
| WP_070446719.1 | Corynebacterium sp. HMSC05D03, |
| | HMSC066C02, and HMSC072D12 |
| WP_070527124.1 | Corynebacterium sp. HMSC074E01 |
| | and HMSC074C01 |
| WP_010190304.1 | C. aurimucosum |
| WP_070719120.1 | Corynebacterium sp. HMSC069E04 |
| WP_070644981.1 | Corynebacterium sp. HMSC062A03 |
| WP_083303801.1 | Corynebacterium sp. HMSC072A02 |
| WP_070683378.1 | Corynebacterium sp. HMSC056E09 |
| WP_046649347.1 | C. minutissimum |
| WP_083299156.1 | Corynebacterium sp. HMSC055A01, |
| | HMSC065D07, and HMSC078C09 |
| WP_016829761.1 | C. diphtheriae |
| WP_010935082.1 | C. diphtheriae |
| WP_045143380.1 | C. diphtheriae |
| WP_003851901.1 | C. diphtheriae |
| WP_072574180.1 | C. diphtheriae |
| WP_071571681.1 | C. diphtheriae |
| OLN14990.1 | C. diphtheriae |
| WP_014319143.1 | C. diphtheriae |
| WP_071572121.1 | C. diphtheriae |
| WP_088246382.1 | C. diphtheriae |
| WP_088266276.1 | C. diphtheriae |
| WP_014302046.1 | C. diphtheriae |
| WP_071575574.1 | C. diphtheriae |
| WP_047262243.1 | C. mustelae |
| WP_048379222.1 | C. renale |
| WP_098389157.1 | C. renale |
| WP_079005470.1 | C. stationis |
| WP_083640700.1 | C. stationis |
| WP_082869553.1 | C. stationis |
| WP_054468244.1 | C. ulcerans |
| WP_015401129.1 | C. halotolerans |
| WP_014010148.1 | C. variable |
| WP_085548717.1 | C. pollutisoli |
| WP_047253276.1 | C. testudinoris |
| WP_040086051.1 | C. humireducens |
| EPD69865.1 | C. pyruviciproducens ATCC BAA-1742 |
| OFO43484.1 | Corynebacterium sp. HMSC073D01 |
| WP_046439708.1 | C. kutscheri |
| WP_006063636.1 | C. durum |
| WP_070434230.1 | Corynebacterium sp. HMSC28B08 |
| WP_025253035.1 | C. vitaeruminis |
| WP_035002300.1 | C. jeikeium |
| WP_025402845.1 | C. falsenii |
| WP_066566197.1 | C. crudilactis |
| WP_053545137.1 | C. deserti |
| WP_042621626.1 | C. marinum |
| WP_055121720.1 | C. oculi |
| WP_026165808.1 | C. mastitidis |
| WP_047240411.1 | C. epidermidicanis |
| WP_082099190.1 | C. camporealensis |
| EEW49517.1 | C. efficiens YS-314 |
| EEG27126.1 | C. matruchotii ATCC 33806 |
| WP_013888590.1 | C. resistens |
| WP_099298604.1 | Corynebacterium sp. Marseille-P4122 |
| WP_055176585.1 | C. lowii |

f. Altered Corynebacterial accDA Proteins

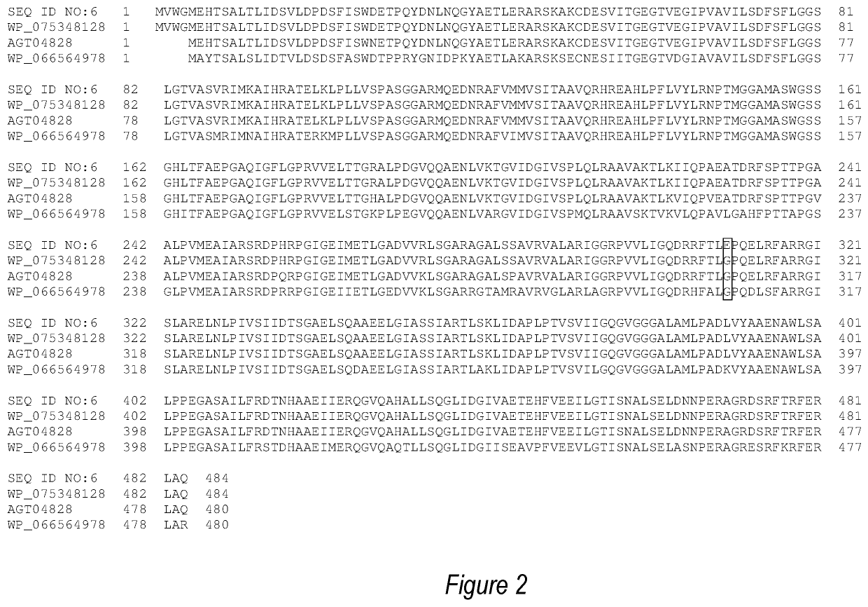

Engineered strains of Corynebacteria comprising any of the structural alterations described above can also comprise an altered corynebacterial accDA protein. Altered corynebacterial accDA proteins comprise a G310E substitution. One example of such a protein is shown in SEQ ID NO: 6. “A corynebacterial accDA protein comprising a G310E substitution” as used herein means an accDA protein from a strain of Corynebacterium which, when compared with SEQ ID NO:6 using the National Center for Biotechnology Information's BLAST® alignment tool, has glutamic acid instead of glycine at the position that aligns with amino acid 310 of SEQ ID NO:6, as illustrated in FIG. 2. Corynebacterial accDA proteins which can be altered to comprise the G310E substitution include, but are not limited to, proteins (regardless of how named) identified with the amino acid sequences provided by the NCBI Reference numbers in Table 2.

TABLE 2
NCBI Reference Numbers of Corynebacterial accDA Proteins

| Reference No. | Species and/or Strain(s) |
|---|---|
| WP_075348128.1 | C. glutamicum AJ1511, ZL-6, ATCC 13869, XV, CP, |
| | and WM001; B. flavum ZL-1 |
| WP_081099520.1 | C. glutamicum |
| WP_077311448.1 | C. glutamicum |
| WP_087061768.1 | C. glutamicum |
| ALP51538.1 | C. glutamicum |
| WP_074508465.1 | C. glutamicum |
| WP_080724089.1 | C. glutamicum |
| WP_003862368.1 | C. glutamicum ATCC 14067, SYPS-062, SYPS-062-33a, |
| | ATCC 21493; B. flavum ATCC 15168 |
| WP_074491484.1 | C. glutamicum |
| WP_011013915.1 | C. glutamicum |
| CAC42827.1 | C. glutamicum |
| WP_081253003.1 | C. glutamicum |
| WP_080506411.1 | C. glutamicum |
| BAF53919.1 | C. glutamicum |
| AMA01529.1 | C. glutamicum |
| SJM44016.1 | C. glutamicum |
| AIK87334.1 | C. glutamicum |
| KEI22570.1 | C. glutamicum ATCC 14067 |
| WP_074508304.1 | C. glutamicum |
| WP_003858326.1 | C. glutamicum SCgG1, SCgG2, S9114, Z188, AS 1.542, |
| | and T6; C. crenatum MT, and SYPA 5-5 |
| OKX89395.1 | C. glutamicum |
| AGT04828.1 | C. glutamicum MB001 |
| ANE09779.1 | C. glutamicum |
| OKX77810.1 | C. glutamicum |
| OKX76530.1 | C. glutamicum |
| WP_066564978.1 | C. crudilactis |
| BAU95211.1 | C. glutamicum |
| WP_096454843.1 | C. glutamicum |
| WP_006770129.1 | C. efficiens |
| WP_053544383.1 | C. deserti |
| WP_047261525.1 | C. mustelae |
| WP_027013104.1 | C. freiburgense |
| WP_075725271.1 | C. aquilae |
| WP_005527314.1 | C. matruchotii |
| WP_080796909.1 | Corynebacterium sp. Marseille-P2417 |
| WP_005523810.1 | C. matruchotii |
| WP_034651919.1 | C. vitaeruminis |
| WP_047239789.1 | C. epidermidicanis DSM 45586 |
| WP_020975778.1 | C. argentoratense |
| WP_084035934.1 | C. glucuronolyticum |
| WP_025252271.1 | C. vitaeruminis |
| WP_005395753.1 | Corynebacterium sp. HMSC073D01; C. glucuronolyticum |
| | ATCC 51866 |
| WP_005391688.1 | C. glucuronolyticum |
| WP_046095662.1 | C. ulcerans |
| WP_046693956.1 | C. ulcerans |
| WP_023635342.1 | C. ulcerans |
| WP_029975236.1 | C. ulcerans |
| WP_044032730.1 | C. ulcerans |
| WP_014525474.1 | C. ulcerans |
| WP_013911032.1 | C. ulcerans |
| WP_046649933.1 | C. xerosis |
| WP_095076119.1 | C. ulcerans |
| WP_038617892.1 | C. ulcerans |
| WP_087453611.1 | C. ulcerans |
| AKN76575.1 | C. ulcerans FRC58 |
| WP_014366686.1 | C. pseudotuberculosis |
| WP_048740779.1 | C. halotolerans |
| WP_014523140.1 | C. pseudotuberculosis |
| WP_032802070.1 | C. pseudotuberculosis |
| WP_071307711.1 | C. pseudotuberculosis |
| WP_013241466.1 | C. pseudotuberculosis |
| WP_014558622.1 | C. pseudotuberculosis |
| WP_048588670.1 | C. pseudotuberculosis |
| WP_014800179.1 | C. pseudotuberculosis |
| WP_070834218.1 | Corynebacterium sp. HMSC073H12 |
| WP_070595314.1 | Corynebacterium sp. HMSC063G05 and HMSC064E08; |
| | C. amycolatum ICIS 9 |
| WP_040426984.1 | C. pyruviciproducens |
| EPD71091.1 | C. pyruviciproducens ATCC BAA-1742 |
| WP_070522630.1 | Corynebacterium sp. HMSC11E11 |
| WP_035120392.1 | C. freneyi |
| WP_070605452.1 | C. sp. HMSC055G02 |
| WP_070565960.1 | Corynebacterium sp. HMSC077G01 and HMSC072D01 |
| WP_070653141.1 | Corynebacterium sp. HMSC065H09 and HMSC072B08 |
| WP_049189720.1 | C. jeikieum 212_CJEI; Corynebacterium |
| | sp. HMSC064E07 |
| WP_070545912.1 | Corynebacterium sp. HMSC064H12, HMSC077C02, |
| | and HMSC070B05 |
| WP_070437593.1 | Corynebacterium sp. HMSC14H10 |
| WP_069359660.1 | C. amycolatum |
| WP_070601703.1 | Corynebacterium sp. HMSC063F04 and HMSC075F02 |
| WP_070642800.1 | Corynebacterium sp. HMSC076C10 |
| WP_070429963.1 | Corynebacterium sp. HMSC14B06 and HMSC077G07 |
| WP_018019334.1 | C. ciconiae |
| WP_016423306.1 | Corynebacterium sp. HFH0082 |
| AFM06966.2 | C. pseudotuberculosis Cp162 |
| WP_038626403.1 | Corynebacterium sp. ATCC 6931 and HMSC11H10 |
| WP_070862586.1 | Corynebacterium sp. HMSC072B09 and HMSC073B01 |
| WP_049171363.1 | C. jeikeium |
| WP_070851988.1 | Corynebacterium sp. HMSC074C03 |
| WP_049180908.1 | C. jeikeium 805_CJEI; Corynebacterium sp. |
| | HMSC074C11, HMSC063A05, and HMSC064E10 |
| WP_049193043.1 | C. jeikeium |
| WP_070855978.1 | Corynebacterium sp. HMSC074C05 |
| WP_088611726.1 | C. jeikeium |
| WP_005511812.1 | C. amycolatum |
| WP_088266872.1 | C. diphtheriae |
| WP_070858287.1 | Corynebacterium sp. HMSC061H03 |
| WP_048758709.1 | C. vitaeruminis |
| WP_072564663.1 | C. diphtheriae |
| WP_014303117.1 | C. diphtheriae |
| WP_014306652.1 | C. diphtheriae |
| WP_070794870.1 | C. diphtheriae |

g. Altered Corynebacterial cg1144 Proteins

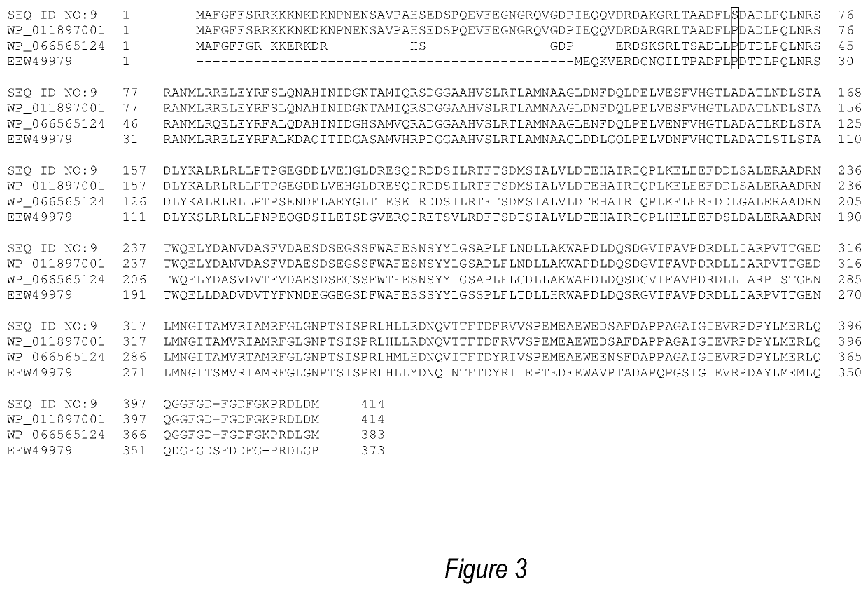

Engineered strains of Corynebacteria comprising any of the structural alterations described above can also comprise an altered corynebacterial cg1144 protein. Altered corynebacterial cg1144 proteins comprise a P66S substitution. One example of such a protein is shown in SEQ ID NO:9. “A corynebacterial cg1144 protein comprising a P66S substitution” as used herein means a cg1144 protein from a strain of Corynebacterium which, when compared with SEQ ID NO:9 using the National Center for Biotechnology Information's BLAST® alignment tool, has serine instead of proline at the position that aligns with amino acid 66 of SEQ ID NO:9, as illustrated in FIG. 3. Corynebacterial cg1144 proteins which can be altered to comprise the P66S substitution include, but are not limited to, proteins (regardless of how named) identified with the amino acid sequences provided by the NCBI Reference numbers in Table 3.

TABLE 3
NCBI Reference Numbers of Corynebacterial cg1144 Proteins

| Reference No. | Species and/or Strains(s) |
|---|---|
| BAB98402.1 | C. glutamicum ATCC 13032 |
| WP_004568112.1 | C. glutamicum 617, ATCC 21493, B Co 03.31, ATCC |
| | 13032 (K051, DSM 20300), MB001, ATCC 14067, AJ1511, CS176, |
| | ZL-6, ATCC 21831, B253, CP, Y1, USDA-ARS-USMARC-56828, C1, |
| | XV, TQ2223, WM001, ATCC 14067, 1006, SYPS-062, SYPS-062-33a, |
| | and AS 1.299; B. flavum 15168 and ZL-1 |
| WP_011897001.1 | C. glutamicum |
| WP_044029870.1 | C. glutamicum |
| WP_074506400.1 | C. glutamicum |
| WP_003856809.1 | C. glutamicum AS 1.542, MT, S9114, SCgG1, SCgG2, |
| | SYPA 5-5, T6-13, and Z188 |
| WP_096455129.1 | C. glutamicum |
| WP_053544501.1 | C. deserti |
| WP_066565124.1 | C. crudilactis |
| WP_011075272.1 | C. efficiens |
| WP_015650827.1 | C. callunae |
| EEW49979.1 | C. efficiens YS-314 |
| WP_080795733.1 | Corynebacterium sp. Marseille-P2417 |
| WP_015400428.1 | C. halotolerans |
| WP_042621053.1 | C. marinum |
| AJK68412.1 | C. marinum DSM 44953 |
| WP_049168821.1 | C. propinquum |
| WP_027017701.1 | C. pseudodiphtheriticum |
| WP_049148216.1 | C. propinquum |
| WP_018119926.1 | C. propinquum |
| WP_023019702.1 | Corynebacterium sp. KPL1995, KPL1989, and |
| | EPI-003-04-2554_SCH2473622 |
| WP_085550290.1 | C. pollutisoli |
| WP_040085436.1 | C. humireducens |
| EKX88113.1 | C. durum F0235 |
| WP_051033899.1 | C. durum |
| WP_020934330.1 | C. maris |
| WP_075814724.1 | Corynebacterium sp. CNJ-954 |
| WP_021353009.1 | C. pseudodiphtheriticum |
| WP_014008877.1 | C. variable |
| WP_052540339.1 | C. glyciniphilum |
| AHW65364.1 | C. glyciniphilum AJ 3170 |
| WP_015400429.1 | C. halotolerans |
| WP_075814720.1 | Corynebacterium sp. CNJ-954 |
| SNV91221.1 | C. urealyticum |
| CUU66362.1 | C. variable |
| WP_020934331.1 | C. maris |
| WP_012361047.1 | C. urealyticum |
| WP_015381984.1 | C. urealyticum |
| WP_071055926.1 | C. jeikeium |
| SCX01561.1 | C. jeikeium |
| WP_049050177.1 | C. jeikeium 931_CJEI; Corynebacterium |
| | sp. HMSC08A12 |
| EEW16595.1 | C. jeikeium ATCC 43734 |
| WP_034965445.1 | C. jeikeium |
| WP_034986706.1 | C. jeikeium |
| WP_011272910.1 | C. jeikeium |
| WP_038550379. | C. glyciniphilum |
| WP_070760374.1 | Corynebacterium sp. HMSC27B11 |
| WP_035004904.1 | C. jeikeium |
| WP_070759388.1 | Corynebacterium sp. HMSC22B11 |
| WP_020440292.1 | C. terpenotabidum |
| WP_066586577.1 | C. provencense |
| WP_013887703.1 | C. resistens |
| WP_010120483.1 | C. nuruki |
| WP_034981842.1 | C. jeikeium |
| WP_034971855.1 | C. jeikeium |
| WP_043012061.1 | C. jeikeium Cj19409; Corynebacterium |
| | sp. HMSC058E07 |
| WP_034969387.1 | C. jeikeium |
| WP_025401902.1 | C. falsenii |
| WP_034974957.1 | C. jeikeium |
| WP_034992651.1 | C. jeikeium |
| WP_035007616.1 | C. jeikeium |
| WP_035002809.1 | C. jeikeium |
| WP_048737023.1 | C. falsenii |
| WP_099298200.1 | Corynebacterium sp. Marseille-P4122 |
| WP_018021816.1 | C. doosanense |
| WP_049168823.1 | C. propinquum |
| WP_049148215.1 | C. propinquum |
| WP_018119927.1 | C. propinquum |
| WP_029157673.1 | C. bovis |
| WP_021353010.1 | C. pseudodiphtheriticum 090104 and DSM 44287; |
| | Corynebacterium sp. KPL1995, |
| | KPL1989, and EPI-003-04-2554_SCH2473622 |
| WP_048737018.1 | C. falsenii |
| WP_047262329.1 | C. mustelae |

h. Insertion of a Promoter in Front of the cg2766 Start Codon

Engineered strains of Corynebacteria comprising any of the structural alterations described above can also comprise a promoter inserted in front of the cg2766 start codon. The inserted promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the inserted promoter is promoter Pcg0007_39 (SEQ ID NO:20); see US 2017/0159045 and WO 2017/00376.

i. Replacement of the Native actA Promoter

Engineered strains of Corynebacteria comprising any of the structural alterations described above can also comprise a replacement actA promoter in place of the native actA promoter. The actA replacement promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the actA replacement promoter is promoter Pcg0007_39 (SEQ ID NO:20).

j. Replacement of the Native opcA Coding Sequence

Engineered strains of Corynebacteria comprising any of the structural alterations described above can also comprise a coding sequence for an opcA protein of a different Corynebacterium species or strain in place of the native opcA coding sequence. Suitable opcA proteins are listed in Table 4A and Table 4B. Using the NCBI's BLAST® alignment tool, the amino acid sequences of the opcA proteins in Table 4A are less than 75% identical to the amino acid sequence of the opcA protein of NRRL B-11474 (SEQ ID NO:94), and the amino acid sequences of the opcA proteins in Table 4B are more than 75% identical to the amino acid sequence of the opcA protein of NRRL B-11474 (SEQ ID NO:94).

In some embodiments the replacement opcA coding sequence is under the control of a promoter that drives expression of both zwf and opcA. In some embodiments, the replacement opcA coding sequence is under the control of an inserted opcA promoter. The inserted opcA promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the inserted opcA promoter is promoter Pcg0007_39 (SEQ ID NO:20).

TABLE 4A
NCBI Reference Numbers for Corynebacterial opcA Proteins

| Reference Number | Species and/or Strain(s) |
|---|---|
| WP_046439480.1 | C. kutscheri |
| WP_099298719.1 | Corynebacterium sp. Marseille-P22 |
| WP_006823740.1 | C. casei UCMA 3821 (GenBank Accession No. |
| | CCE56308.1), LMG S-19264; Corynebacterium |
| | sp. JB4; SEQ ID NO: 92 |
| WP_095661089.1 | C. glaucum |
| WP_095276461.1 | Corynebacterium sp. NBT06-6, NML99-0020, |
| | and NML92-0415 |
| WP_095553723.1 | Corynebacterium sp. NML 150383 |
| WP_095548171.1 | Corynebacterium sp. NML00-0156 |
| WP_095545764.1 | Corynebacterium sp. NML 120412 |
| WP_095536183.1 | Corynebacterium sp. NML93-0607 |
| SNV73072.1 | C. imitans |
| WP_092286146.1 | C. spheniscorum |
| WP_092255258.1 | C. cystitidis |
| WP_092148525.1 | C. mycetoides |
| WP_088266936.1 | C. diphtheriae |
| WP_088265045.1 | C. diphtheriae |
| WP_088246432.1 | C. diphtheriae |
| WP_087454020.1 | C. ulcerans |
| WP_087117967.1 | C. urinapleomorphum |
| WP_049360793.1 | C. aurimucosum 1237_CAUR; C. kefirresidentii SB |
| WP_086891441.1 | C. striatum |
| WP_085958015.1 | C. fournierii |
| WP_085713610.1 | C. diphtheriae |
| WP_085549995.1 | C. pollutisoli |
| SLM96782.1 | C. xerosis |
| WP_084759291.1 | C. phoceense |
| WP_084168239.1 | C. atypicum |
| WP_084082961.1 | C. freneyi |
| WP_082796495.1 | C. variabile |
| WP_081920634.1 | C. variabile |
| WP_081803842.1 | C. glyciniphilum |
| WP_078057488.1 | C. provencense |
| WP_076598597.1 | C. appendicis |
| WP_075816106.1 | Corynebacterium sp. CNJ-954 |
| WP_075734014.1 | C. phocae |
| WP_075729852.1 | C. flavescens |
| WP_075726270.1 | C. aquilae |
| WP_075722895.1 | C. stationis |
| WP_075691979.1 | C. sphenisci |
| WP_075663775.1 | C. frankenforstense |
| WP_072590090.1 | C. diphtheriae |
| WP_071575625.1 | C. diphtheriae |
| WP_071572602.1 | Corynebacterium sp. NML120713 |
| WP_071571645.1 | C. diphtheriae |
| WP_071570066.1 | C. diphtheriae |
| WP_071568288.1 | Corynebacterium sp. NML140438 |
| WP_071566266.1 | Corynebacterium sp. NML130628 |
| WP_071056558.1 | C. jeikeium |
| WP_070976878.1 | Corynebacterium sp. MC3 |
| WP_070832090.1 | Corynebacterium sp. HMSC073H12 and HMSC074C03 |
| WP_070681225.1 | Corynebacterium sp. HMSC072A04, HMSC074H12, |
| | and HMSC035E02 |
| WP_070653088.1 | Corynebacterium sp. HMSC065H09 and HMSC072B08 |
| WP_070451416.1 | C. coyleae DSM 44184; Corynebacterium |
| | sp. HMSC11D10 and HMSC034E211 |
| WP_070866373.1 | Corynebacterium sp. HMSC072D01 |
| WP_070856734.1 | Corynebacterium sp. HMSC074C05 |
| WP_070843904.1 | Corynebacterium sp. HMSC034A01 |
| WP_070839048.1 | Corynebacterium sp. HMSC070H05 |
| WP_070828994.1 | Corynebacterium sp. HMSC036E10 |
| WP_070822776.1 | Corynebacterium sp. HMSC036D03 |
| WP_070816208.1 | Corynebacterium sp. HMSC034B08 |
| WP_070799051.1 | C. diphtheriae |
| WP_070770986.1 | Corynebacterium sp. HMSC075D04 |
| WP_070768006.1 | Corynebacterium sp. HMSC04H06 |
| WP_070761406.1 | Corynebacterium sp. HMSC27B11 |
| WP_070758918.1 | Corynebacterium sp. HMSC22B11 |
| WP_070738933.1 | Corynebacterium sp. HMSC073D01 |
| WP_070732205.1 | Corynebacterium sp. HMSC076D02 |
| WP_070683023.1 | Corynebacterium sp. HMSC056E09 |
| WP_070671164.1 | Corynebacterium sp. HMSC078H07 |
| WP_070643476.1 | Corynebacterium sp. HMSC062A03 and HMSC072D12 |
| WP_070642957.1 | Corynebacterium sp. HMSC076C10 |
| WP_070615011.1 | Corynebacterium sp. HMSC067D03 |
| WP_070570372.1 | Corynebacterium sp. HMSC055D05 |
| WP_070567126.1 | C. jeikeium FDAARGOS_328; Corynebacterium |
| | sp. HMSC077G01, HMSC063F04, HMSC064E07, HMSC072B09, and HMSC073B01 |
| WP_070564456.1 | Corynebacterium sp. HMSC072A02 and HMSC078A10 |
| WP_070549602.1 | Corynebacterium sp. HMSC077G07 |
| WP_070547341.1 | Corynebacterium sp. HMSC070E08 |
| WP_070536503.1 | Corynebacterium sp. HMSC05E07 |
| WP_070528425.1 | Corynebacterium sp. HMSC074E01, HMSC074C01, |
| | and HMSC036D02 |
| WP_070524283.1 | Corynebacterium sp. HMSC055A01, HMSC065D07, |
| | and HMSC078C09 |
| WP_070522356.1 | Corynebacterium sp. HMSC11E11 |
| WP_070494917.1 | Corynebacterium sp. HMSC30G07 |
| WP_070487506.1 | Corynebacterium sp. HMSC08A12 |
| WP_070484826.1 | Corynebacterium sp. HMSC08F01 |
| WP_070473987.1 | Corynebacterium sp. HMSC05H05 |
| WP_070462224.1 | Corynebacterium sp. HMSC29G08 |
| WP_070444875.1 | Corynebacterium sp. HMSC055A01, HMSC065D07, |
| | and HMSC078C09 |
| WP_070435059.1 | Corynebacterium sp. HMSC28B08 |
| WP_070423128.1 | Corynebacterium sp. HMSC05C01 |
| WP_070420257.1 | Corynebacterium sp. HMSC06C06 |
| WP_070362427.1 | Corynebacterium sp. NML98-0116 and HMSC08D02 |
| WP_062035107.1 | C. simulans PES1; Corynebacterium sp. HMSC06G04 |
| WP_061922333.1 | C. simulans Wattiau and 1B08; Corynebacterium |
| | sp. HMSC06D04, HMSC08C04, HMSC077D03, HMSC071F07; |
| | Corynebacterium accolens AH4003 |
| WP_049193712.1 | C. jeikeium 401_CJE1; Corynebacterium |
| | sp. HMSC055G02 |
| WP_046649108.1 | C. aurimucosum 944_CAUR; Corynebacterium |
| | sp. HMSC068G04 and HMSC056F09; C. minutissimum 1941 |
| WP_043015176.1 | C. jeikeium Cj30184; Corynebacterium |
| | sp. HMSC058E07 |
| WP_005531605.1 | C. striatum 216, ATCC 6940, 797_CAUR, 587_CAUR, |
| | 542_CAUR, 1329_CAUR, 1327_CAUR, 2230, 2245, 2023, and 2247; |
| | Corynebacterium sp. HMSC05D08 |
| OHF39428.1 | Corynebacterium sp. HMSC074A01 |
| WP_066493224.1 | Corynebacterium sp. DNF00584, CMW7794, |
| | HMSC072G08, HMSC077B05, HMSC077D10, and HMSC0665A05 |
| WP_066839768.1 | C. stationis |
| WP_066794092.1 | C. stationis |
| WP_066525378.1 | C. bouchesdurhonense |
| WP_049167646.1 | C. propinquum |
| WP_049158908.1 | C. striatum |
| WP_049148304.1 | C. propinquum |
| WP_065421559.1 | C. halotolerans |
| WP_064833329.1 | Corynebacterium sp. EPI-003-04-2554_SCH2473622 |
| WP_063936745.1 | C. afermentans |
| WP_058832265.1 | C. pseudotuberculosis |
| WP_006063324.1 | C. durum |
| WP_058093366.1 | C. pseudotuberculosis |
| WP_055178564.1 | C. lowii |
| WP_055121854.1 | C. oculi |
| AKK06005.1 | C. mustelae |
| WP_053412142.1 | C. lactis |
| WP_052204189.1 | C. riegelii |
| WP_038628980.1 | C. jeikeium 805_CJEI; Corynebacterium sp. |
| | ATCC 6931, HMSC11H10, HMSC074C11, HMSC063A05, HMSC064E10, |
| | and HMSC074C04 |
| WP_049049964.1 | C. jeikeium |
| WP_049378186.1 | C. aurimucosum |
| WP_048758558.1 | C. vitaeruminis |
| WP_048737982.1 | C. falsenii |
| WP_016422736.1 | C. jeikeium 239_CJEI and 212_CJEI; C. urealyticum |
| | 1055_CURE; Corynebacterium sp. HFH0082, HMSC14H10, |
| | HMSC064H12, HMSC063G05, HMSC077C02, HMSC064E08, HMSC070B05, |
| | HMSC061H03, HMSC075F02; C. amycolatum_ICIS |
| | 53 and ICIS 9 |
| WP_048381636.1 | C. renale |
| WP_047263492.1 | C. mustelae |
| WP_047259618.1 | C. uterequi |
| WP_047253131.1 | C. testudinoris |
| WP_047240275.1 | C. epidermidicanis |
| WP_005526431.1 | C. matruchotii |
| WP_046650939.1 | C. xerosis |
| WP_046645947.1 | C. striatum |
| WP_046204153.1 | C. argentoratense |
| WP_046202297.1 | C. kroppenstedtii |
| WP_046095943.1 | C. ulcerans |
| AHW64140.1 | C. glyciniphilum AJ 3170 |
| WP_042621478.1 | C. marinum |
| WP_042530750.1 | C. singulare |
| WP_042404782.1 | C. jeddahense |
| WP_040354603.1 | C. ammoniagenes |
| WP_040085900.1 | C. humireducens (GenBank Accession No. AJE33249.1, |
| | SEQ ID NO: 25) |
| WP_039675253.1 | C. minutissimum |
| WP_038620584.1 | C. ulcerans |
| WP_038611699.1 | C. ureicelerivorans |
| WP_035113157.1 | C. auriscanis |
| WP_035104698.1 | C. camporealensis |
| WP_035012334.1 | C. jeikeium |
| WP_035010427.1 | C. jeikeium |
| WP_035008179.1 | C. jeikeium |
| WP_035003885.1 | C. jeikeium |
| WP_035001868.1 | C. jeikeium |
| WP_034998920.1 | C. ihumii |
| WP_034987985.1 | C. jeikeium |
| WP_034981960.1 | C. jeikeium |
| WP_034974411.1 | C. jeikeium |
| WP_034967970.1 | C. jeikeium |
| WP_034663424.1 | C. tuscaniense |
| WP_027020497.1 | C. sputi |
| WP_027012302.1 | C. freiburgense |
| WP_025402713.1 | C. falsenii (GenBank Accession No. AHI03141.1), |
| | SEQ ID NO: 89 |
| WP_025252883.1 | C. vitaeruminis (GenBank Accession No. AHI22863.1); |
| | SEQ ID NO: 87 |
| WP_023022439.1 | Corynebacterium sp. KPL1859, KPL1855, and KPL1814 |
| WP_023021500.1 | Corynebacterium sp. KPL1860, KPL1857, KPL1856, |
| | KPL1821, KPL1817, and KPL1856 |
| WP_023019439.1 | Corynebacterium sp. KPL1995 and KPL1989 |
| WP_023017462.1 | Corynebacterium sp. KPL2004, KPL1998, KPL1996, |
| | KPL1986, KPL1824, and KPL1996 |
| WP_005279118.1 | C. accolens ATCC 49725; Corynebacterium |
| | sp. KPL1818 |
| WP_022862033.1 | C. massiliense |
| WP_021353956.1 | C. pseudodiphtheriticum |
| WP_020976309.1 | C. argentoratense |
| WP_005324852.1 | C. tuberculostearicum SK141; |
| | C. pseudogenitalium ATCC 33035 |
| WP_020934820.1 | C. maris |
| WP_020441165.1 | C. terpenotabidum (GenBank Accession No. AGP30804.1), |
| | SEQ ID NO: 93 |
| WP_019194592.1 | C. timonense |
| WP_018582009.1 | C. pilosum |
| WP_018339807.1 | C. caspium |
| WP_018295562.1 | C. lubricantis |
| WP_018119593.1 | C. propinquum |
| WP_018118857.1 | C. mastitidis |
| WP_018023988.1 | C. ulceribovis |
| WP_018021704.1 | C. doosanense |
| WP_018019762.1 | C. ciconiae |
| WP_018017013.1 | C. capitovis |
| WP_016457552.1 | C. pyruviciproducens (GenBank Accession No. EPD70204.1), |
| | SEQ ID NO: 91 |
| WP_010265064.1 | C. bovis |
| WP_010186759.1 | C. aurimucosum |
| EFG82419.1 | C. ammoniagenes DSM 20306 |
| WP_006840267.1 | C. lipophiloflavum |
| WP_005521071.1 | C. matruchotii (GenBank Accession No. EEG26974.1), |
| | SEQ ID NO: 88 |
| WP_005510779.1 | C. amycolatum |
| WP_005389976.1 | C. glucuronolyticum |
| WP_005294809.1 | C. jeikeium |
| WP_005290056.1 | C. genitalium |
| WP_005283075.1 | C. accolens |
| WP_015400950.1 | C. halotolerans (GenBank Accession No. AGF72531.1), |
| | SEQ ID NO: 90 |
| WP_014525815.1 | C. ulcerans |
| WP_014367098.1 | C. pseudotuberculosis |
| WP_014306922.1 | C. diphtheriae |
| WP_014009994.1 | C. variabile |
| WP_013911577.1 | C. ulcerans |
| WP_013888447.1 | C. resistens |
| WP_013241959.1 | C. pseudotuberculosis |
| WP_012731602.1 | C. kroppenstedtii |
| WP_012360244.1 | C. urealyticum |
| WP_011273572.1 | C. jeikeium |
| WP_003851588.1 | C. diphtheriae |

TABLE 4B
NCBI Reference Numbers for Corynebacterial OpcA Proteins

| Reference Number | Species and/or Strain |
|---|---|
| WP_075348233.1 | C. glutamicum XV |
| WP_003862265.1 | C. glutamicum ATCC 14067, ATCC 21831, AR1, |
| | USDA-ARS-USMARC-56828, YI, SYPS-062, SYPS-062-33a, and |
| | ATCC 21493; B. flavum ATCC 15168 |
| WP_040967467.1 | C. glutamicum |
| WP_003856048.1 | C. glutamicum SCgG1, SCgG2, S9114, Z118, |
| | CS176, AS 1.542, TG-13; C. crenatum MT |
| WP_074508163.1 | C. glutamicum |
| WP_011897297.1 | C. glutamicum |
| WP_011014458.1 | C. glutamicum |
| WP_087497587.1 | C. glutamicum |
| WP_066565817.1 | C. crudilactis |
| WP_096456023.1 | C. glutamicum |
| WP_053544982.1 | C. deserti |
| WP_015651366.1 | C. callunae |
| WP_080794830.1 | Corynebacterium sp. Marseille-P2417 |
| WP_006767698.1 | C. efficiens |

k. Insertion of at Least One Additional Copy of lysA, ask, asd, ddh, and/or dapB

Engineered strains of Corynebacteria comprising any of the structural alterations described above can also comprise at least one additional copy of at least one gene selected from the group consisting of lysA, ask, asd, ddh, and dapB. The additional copies can be provided using one or more vectors. Example 1 describes two integration vectors that can be used to insert copies of ask, asd, ddh, and dapB.

In some embodiments, the engineered Corynebacterium contains at least one additional copy of lysA, ask, asd, ddh, and dapB.

In some embodiments, the engineered Corynebacterium contains at least one additional copy of ask, asd, ddh, and dapB. In other embodiments, the engineered Corynebacterium contains at least one additional copy of lysA, asd, ddh, and dapB. In other embodiments, the engineered Corynebacterium contains at least one additional copy of lysA, ask, ddh, and dapB. In other embodiments, the engineered Corynebacterium contains at least one additional copy of lysA, ask, asd, and dapB. In other embodiments, the engineered Corynebacterium contains at least one additional copy of lysA, ask, asd, and ddh.

In some embodiments, the engineered Corynebacterium contains at least one additional copy of asd, ddh, and dapB. In other embodiments, the engineered Corynebacterium contains at least one additional copy of ask, ddh, and dapB. In other embodiments, the engineered Corynebacterium contains at least one additional copy of ask, asd, and dapB. In other embodiments, the engineered Corynebacterium contains at least one additional copy of ask, asd, and ddh. In other embodiments, the engineered Corynebacterium contains at least one additional copy of lysA, ddh, and dapB. In other embodiments, the engineered Corynebacterium contains at least one additional copy of lysA, asd, and dapB. In other embodiments, the engineered Corynebacterium contains at least one additional copy of lysA, asd, and ddh. In other embodiments, the engineered Corynebacterium contains at least one additional copy of lysA, ask, and dapB. In other embodiments, the engineered Corynebacterium contains at least one additional copy of lysA, ask, and ddh.

In some embodiments, the engineered Corynebacterium contains at least one additional copy of ddh and dapB. In other embodiments, the engineered Corynebacterium contains at least one additional copy of asd and dapB. In other embodiments, the engineered Corynebacterium contains at least one additional copy of asd and ddh. In other embodiments, the engineered Corynebacterium contains at least one additional copy of lysA and dapB. In other embodiments, the engineered Corynebacterium contains at least one additional copy of lysA and ddh. In other embodiments, the engineered Corynebacterium contains at least one additional copy of lysA, and ask.

In other embodiments, the engineered Corynebacterium contains at least one additional copy of lysA. In other embodiments, the engineered Corynebacterium contains at least one additional copy of ask. In other embodiments, the engineered Corynebacterium contains at least one additional copy of asd. In other embodiments, the engineered Corynebacterium contains at least one additional copy of ddh. In other embodiments, the engineered Corynebacterium contains at least one additional copy of dapB.

In any of the embodiments described above in which the engineered Corynebacterium contains at least one additional copy of lysA, the additional copy of lysA is a codon-optimized version described below.

l. Replacement of the Start Codon of aceE

In some embodiments, the start codon of the native aceE gene in the genome of any of the engineered strains of Corynebacteria described above is replaced. In some of these embodiments, the start codon is replaced with GTG, CTG, or TTG. In some embodiments, the start codon is replaced with TTG.

m. Replacement of the Native pyc Gene Promoter

Engineered strains of Corynebacteria comprising any of the structural alterations described above can also comprise a pyc replacement promoter in place of the native pyc gene promoter. The pyc replacement promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the pyc replacement promoter is promoter Pcg1860 (SEQ ID NO:11; see US 2017/0159045).

n. Insertion of a Promoter in Front of the zwf Open Reading Frame

Engineered strains of Corynebacteria comprising any of the structural alterations described above can also comprise a promoter inserted in front of the zwf open reading frame. The inserted promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. In some embodiments, the additional promoter is promoter Pcg0007_39 (SEQ ID NO:20).

o. Insertion of a Codon-Optimized lysA Coding Sequence

Engineered strains of Corynebacteria comprising any of the structural alterations described above can comprise a codon-optimized lysA coding sequence under the control of a promoter and including terminator sequence. The promoter can be a promoter from a different gene of the Corynebacterium species or strain being engineered or can be a heterologous promoter (i.e., a promoter of another Corynebacterium species or strain or an artificially constructed promoter). These promoters include, but are not limited to, promoters disclosed in Nešvera et al., 2012; Pátek et al., 2003(a); Pátek et al., 2003(b); Pátek et al., 2013; Rytter et al., 2014; Shang et al., 2017; Yim et al., 2013; US 2017/0159045; and WO 2017/00376. Terminator sequences include, but are not limited to, those disclosed in Pfeifer-Sancar et al., 2013.

In some embodiments, the promoter is Pcg0007_39 (SEQ ID NO:20). In some of these embodiments, the codon-optimized sequence is SEQ ID NO:16. In some of these embodiments, the terminator is the sod terminator (nucleotides 1436-1516 of SEQ ID NO:17). In some embodiments, the genome of the engineered Corynebacterium contains the nucleotide sequence SEQ ID NO:17, which is codon-optimized lysA sequence SEQ ID NO:16 under the control of promoter Pcg0007_39 (SEQ ID NO:20) and having a sod terminator (nucleotides 1436-1516 of SEQ ID NO:17).

### Nucleic Acids and Vectors

Vectors can be used to engineer a Corynebacterium having one or more of the structural alterations described above, resulting in improved lysine production compared with the corresponding native Corynebacterium (i.e., the Corynebacterium which has not been engineered to include the structural alterations). Such corynebacteria include, but are not limited to, the Corynebacterium deposited as NRRL B-11474, the Corynebacterium deposited as NRRL B-67439, and the corynebacterial species and strains in Tables 1, 2, 3, 4A, and 4B. Methods of delivering vectors to Corynebacteria are well known and include, for example, calcium phosphate transfection, DEAE-dextran mediated transfection, microinjection, lipid-mediated transfection, electroporation, conjugation, and infection.

Nucleic acids that can be used to engineer the alterations described above are well known and are described, for example, in U.S. Pat. Nos. 7,368,276, 6,927,046, US 2017/0159045, and WO 2017/00376 and in the Examples, below. Nucleic acids encoding the altered corynebacterial rnaJ, accDA, and cg1144 proteins described above can be included in vectors in which a coding sequence is operably linked to a suitable regulatory sequence for expression in a Corynebacterium. SEQ ID NO:2, SEQ ID NO:5, and SEQ ID NO:8 are examples of nucleotide sequences encoding SEQ ID NO:3, SEQ ID NO:6, and SEQ ID NO:9, respectively, but any nucleotide sequence that encodes the altered corynebacterial protein can be used. The nucleotide sequences can be optimized for expression in various species or strains of Corynebacteria as is well known in the art.

The regulatory sequence includes a suitable mRNA ribosome binding site and a sequence for regulating the termination of transcription and translation and may include other elements, such as a promoter or operator. Once transformed into a host Corynebacterium, the vector may replicate or function independently of the host genome or may integrate into the genome itself. The vector that is used is not specifically limited and may be any vector known in the art, as long as it can replicate in a Corynebacterium host. See, for example, Lee, 2014; Knoppová et al., 2007; and Pátek & Nešvera, 2013.

A vector can include at least one selectable marker, such as an antibiotic resistance gene. Suitable antibiotics include, e.g., amikacin, ampicillin, augmentin (amoxicillin plus clavulonic acid), cefazolin, cefoxitin, ceftazidime, ceftiofur, cephalothin, chloramphenicol, enrofloxacin, florfenicol, gentamicin, imipenem, kanamycin, penicillin, sarafloxicin, spectinomycin, streptomycin, tetracycline, ticarcillin, and tilmicosin.

### Engineered Strains of Corynebacteria

This disclosure provides strains of Corynebacteria engineered to contain at least one of the four alterations described above—i.e., (a) insertion of a replacement promoter in front of the cg1383 start codon; (b) replacement of the native phoU promoter; (c) replacement of the native cg3210 promoter; and (d) replacement of the native cg0800 promoter—as well as at least one of the following alterations:
(e) alteration of the rnaJ coding sequence to encode a ribonuclease J protein comprising a G448S substitution;
(f) alteration of the native accDA coding sequence to encode an acetyl-CoA carboxylase carboxyl transferase subunit alpha/beta protein comprising a G310E substitution;
(g) alteration of the native cg1144 coding sequence to encode a cg1144 protein comprising a P66S substitution;
(h) insertion of a promoter in front of the cg2766 start codon;
(i) replacement of the native actA promoter;
(j1) replacement of the native opcA coding sequence by coding sequence for an opcA protein of a different Corynebacterium species or strain;
(j2) replacement of the native opcA coding sequence by coding sequence for an opcA protein of a different Corynebacterium species or strain under the control of an inserted opcA promoter;
(k) insertion of at least one additional copy of at least one gene selected from the group consisting ask, asd, ddh, and dapB;
(l) replacement of the start codon of the native aceE gene;
(m) replacement of the native pyc gene promoter;
(n) insertion of an additional promoter in front of the zwf open reading frame; and
(o) insertion of a codon-optimized lysA coding sequence.

The following non-limiting embodiments of engineered Corynebacteria fall within the description above.

Embodiment 1. The Corynebacterium deposited with the Agriculture Research Culture Collection (NRRL) International Depository Authority, 1815 N. University Street, Peoria, IL 61604 on Dec. 22, 2017, under the provisions of the Budapest Treaty and assigned Accession No. NRRL B-67535.

Embodiment 2. A Corynebacterium having a bacterial genome consisting essentially of the genome of the bacterium deposited as NRRL B-67439 but for up to 4 structural alterations selected from the group consisting of:
(a) insertion of a promoter in front of the cg1383 start codon;
(b) replacement of the native phoU promoter;
(c) replacement of the native cg3210 promoter; and
(d) replacement of the native cg0800 promoter.

Embodiment 3. A Corynebacterium having a bacterial genome consisting essentially of the genome of the bacterium deposited as NRRL B-11474 but for
(i) at least one structural alteration selected from the group consisting of:
   (a) insertion of a promoter in front of the cg1383 start codon;
   (b) replacement of the native phoU promoter;
   (c) replacement of the native cg3210 promoter; and
   (d) replacement of the native cg0800 promoter; and
(ii) at least one additional structural alteration selected from the group consisting of:
   (e) alteration of the rnaJ coding sequence to encode a corynebacterial rnaJ protein comprising a G448S substitution;
   (f) alteration of the native accDA coding sequence to encode a corynebacterial accDA protein comprising a G310E substitution;
   (g) alteration of the native cg1144 coding sequence to encode a corynebacterial cg1144 protein comprising a P66S substitution;
   (h) insertion of a promoter in front of the cg2766 start codon;
   (i) replacement of the native actA promoter;
   (j1) replacement of the native opcA coding sequence by a replacement opcA coding sequence for an opcA protein of a different Corynebacterium;
   (j2) replacement of the native opcA coding sequence by a replacement opcA coding sequence for an opcA protein of a different Corynebacterium under the control of an inserted opcA promoter;
   (k) insertion of at least one additional copy of at least one gene selected from the group consisting of lysA, ask, asd, ddh, and dapB;
   (l) replacement of the native start codon of aceE by GTG, CTG, or TTG;
   (m) replacement of the native pyc gene promoter by promoter Pcg1860 (SEQ ID NO:11);
   (n) insertion of promoter Pcg0007_39 (SEQ ID NO:20) in front of the zwf open reading frame; and
   (o) insertion of a codon-optimized lysA coding sequence.

Embodiment 4. The Corynebacterium of Embodiment 2 or 3, wherein the promoter in front of the cg1383 start codon is promoter Pcg0007_39 (SEQ ID NO:20).

Embodiment 5. The Corynebacterium of any of Embodiments 2-4, wherein the native phoU promoter is replaced by promoter Pcg0007_39 (SEQ ID NO:20).

Embodiment 6. The Corynebacterium of any of Embodiments 2-5, wherein the native cg3210 promoter is replaced by promoter Pcg0007_39 (SEQ ID NO:20).

Embodiment 7. The Corynebacterium of any of Embodiments 2-6, wherein the native cg0800 promoter is replaced by promoter Pcg0007_39 (SEQ ID NO:20).

Embodiment 8. The Corynebacterium of any of Embodiments 3-7, in which (e) the native rnaJ coding sequence is altered to encode the corynebacterial rnaJ protein comprising a G448S substitution.

Embodiment 9. The Corynebacterium of any of Embodiments 3-8, in which (e) the native rnaJ coding sequence is altered to encode the amino acid sequence SEQ ID NO:3.

Embodiment 10. The Corynebacterium of any of Embodiments 3-9, in which (f) the native accDA coding sequence is altered to encode the corynebacterial accDA protein comprising the G310E substitution.

Embodiment 11. The Corynebacterium bacterium of any of Embodiments 3-10, in which (f) the native accDA coding sequence is altered to encode the amino acid sequence SEQ ID NO:6.

Embodiment 12. The Corynebacterium of any of Embodiments 3-11, in which (g) the native cg1144 coding sequence is altered to encode the corynebacterial cg1144 protein comprising the P66S substitution.

Embodiment 13. The Corynebacterium bacterium of any of Embodiments 3-12, in which (g) the native cg1144 coding sequence is altered to encode the amino acid sequence SEQ ID NO:9.

Embodiment 14. The Corynebacterium bacterium of any of Embodiments 3-13, in which (h) the promoter is inserted in front of the cg2766 start codon.

Embodiment 15. The Corynebacterium of any of Embodiments 3-14, in which (h) the promoter inserted in front of the cg2766 start codon is promoter Pcg0007_39 (SEQ ID NO:20).

Embodiment 16. The Corynebacterium bacterium of any of Embodiments 3-15, in which (i) the native actA promoter is replaced.

Embodiment 17. The Corynebacterium of any of Embodiments 3-16 in which (i) the native actA promoter is replaced by promoter Pcg0007_39 (SEQ ID NO:20).

Embodiment 18. The Corynebacterium bacterium of any of Embodiments 3-17, in which (j) the native opcA coding sequence is replaced by a replacement opcA coding sequence for an opcA protein of a different Corynebacterium.

Embodiment 19. The Corynebacterium of any of Embodiments 3-18, in which the native opcA coding sequence is replaced by a replacement opcA coding sequence for an opcA protein of a Corynebacterium selected from the group consisting of Corynebacterium vitaeruminis DSM 20294, Corynebacterium matruchotii ATCC 33806, Corynebacterium falsenii DSM 44353, Corynebacterium halotolerans YIM 70093, Corynebacterium pyruviciproducens ATCC BAA-1742, Corynebacterium casei UCMA 3821, Corynebacterium terpenotabidum Y-11, and C. humireducens NBRC 106098.

Embodiment 20. The Corynebacterium of any of Embodiments 3-19, in which the native opcA coding sequence is replaced by a replacement opcA coding sequence for an opcA protein of C. humireducens NBRC 106098.

Embodiment 21. The Corynebacterium of any of Embodiments 3-20, in which the replacement opcA coding sequence is under control of an inserted opcA promoter.

Embodiment 22. The Corynebacterium of Embodiment 21, in which the inserted opcA promoter is promoter Pcg0007_39 (SEQ ID NO:20).

Embodiment 23. The Corynebacterium bacterium of any of Embodiments 3-22, in which (k) the genome of the bacterium contains the at least one additional copy of the at least one gene selected from the group consisting of lysA, ask, asd, ddh, and dapB.

Embodiment 24. The Corynebacterium bacterium of Embodiment 23, in which the genome of the bacterium contains (a) at least one additional copy of lysA, ask, asd, ddh, and dapB.

Embodiment 24. The Corynebacterium bacterium of Embodiment 23, in which the genome of the bacterium contains:
(b) at least one additional copy of ask, asd, ddh, and dapB;
(c) at least one additional copy of lysA, asd, ddh, and dapB;
(d) at least one additional copy of lysA, ask, ddh, and dapB;
(e) at least one additional copy of lysA, ask, asd, and dapB; or
(f) at least one additional copy of lysA, ask, asd, and ddh.

Embodiment 25. The Corynebacterium bacterium of Embodiment 23, in which the genome of the bacterium contains:
(g) at least one additional copy of asd, ddh, and dapB;
(h) at least one additional copy of ask, ddh, and dapB;
(i) at least one additional copy of ask, asd, and dapB;
(j) at least one additional copy of ask, asd, and ddh;
(k) at least one additional copy of lysA, ddh, and dapB;
(l) at least one additional copy of lysA, asd, and dapB;
(m) at least one additional copy of lysA, asd, and ddh;
(n) at least one additional copy of lysA, ask, and dapB; or
(o) at least one additional copy of lysA, ask, and ddh.

Embodiment 26. The Corynebacterium bacterium of Embodiment 23, in which the genome of the bacterium contains:
(p) at least one additional copy of ddh and dapB;
(q) at least one additional copy of asd and dapB;
(r) at least one additional copy of asd and ddh;
(s) at least one additional copy of lysA and dapB;
(t) at least one additional copy of lysA and ddh;
(u) at least one additional copy of lysA, and ask.

Embodiment 27. The Corynebacterium bacterium of Embodiment 23, in which the genome of the bacterium contains:
(v) at least one additional copy of lysA;
(w) at least one additional copy of ask;
(x) at least one additional copy of asd;
(y) at least one additional copy of ddh; or
(z) at least one additional copy of dapB.

Embodiment 28. The Corynebacterium bacterium of any of Embodiments 3-27, in which the start codon of aceE is replaced by TTG, GTG, or CTG.

Embodiment 29. The Corynebacterium bacterium of any of Embodiments 3-28, in which the start codon of aceE is replaced by TTG.

Embodiment 30. The Corynebacterium bacterium of any of Embodiments 3-29, in which the native pyc gene promoter is replaced.

Embodiment 31. The Corynebacterium bacterium of any of Embodiments 3-30, in which the native pyc gene promoter is replaced by promoter Pcg1860 (SEQ ID NO:11).

Embodiment 32. The Corynebacterium bacterium of any of Embodiments 3-31, in which a promoter is inserted in front of the zwf open reading frame.

Embodiment 33. The Corynebacterium bacterium of any of Embodiments 3-32, in which promoter Pcg0007_39 (SEQ ID NO:20) is inserted in front of the zwf open reading frame.

Embodiment 34. The Corynebacterium of any of Embodiments 3-33, which contains (o) a codon-optimized lysA coding sequence.

Embodiment 35. The Corynebacterium of Embodiment 34, in which the codon-optimized lysA coding sequence is under control of Pcg0007_39 (SEQ ID NO:20).

Embodiment 36. The Corynebacterium of Embodiment 34 or 35, which comprises a heterologous terminator for the codon-optimized lysA coding sequence.

Embodiment 37. The Corynebacterium bacterium of any of Embodiments 34-36, which comprises the nucleotide sequence SEQ ID NO:17.

### Production of Lysine

Methods of using Corynebacteria to produce lysine are well known in the art, and the engineered Corynebacteria provided in this disclosure can be used with any of these methods. See, for example, U.S. Pat. Nos. 8,048,649, 7,635,579, 7,504,242, 7,300,777, 7,267,967, 7,160,711, 7,141,388, 7,122,369, 6,927,046, 6,830,903, US 2002/0086371, US 2004/0043458, US 2009/0325244, EP 1246921, and EP 1619252.

Those skilled in the art will appreciate that there are numerous variations and permutations of the above described embodiments that fall within the scope of the appended claims.

### Example 1. Preparation of L-Lysine Pathway Four-Gene Constructs

### Example 1A. Construction of an Integration Vector Containing Lysine Biosynthesis Genes ask-asd-dapB-ddh in the bioD Region

pBKMS vector is a pBR322 derivative unable to replicate in C. glutamicum and which contains a kanamycin resistance gene marker and a levansucrase sacB gene from Bacillus subtilis under the control of a strong synthetic promoter for sucrose counter-selection. The 5.4 kb ask-asd-ddh-dapB-orj2′ cassette (4Go) was digested from pFC3-ask-asd-dapB-ddh plasmid (U.S. Pat. No. 7,368,276) with PmeI and XmaI and ligated into pDElia11 (U.S. Pat. No. 6,927,046) linearized with HincII and XmaI to construct pD11-KBDH.

To generate a 9.4 kb homology region containing a SpeI site, a DNA fragment was amplified from NRRL B-11474 genomic DNA using primers 18417 (SEQ ID NO:70) and 184F8 (SEQ ID NO:71) and cloned into the pBKMS vector NdeI/PstI sites by IN-FUSION® (Clontech). The resulting plasmid pBKMS 184 3p was digested with SpeI and a second PCR product similarly obtained from NRRL B-11474 genomic DNA using primers 161-184f5 and 162-184r6 was inserted by IN-FUSION® reaction to generate pBKMS184. The 5.4 kb 4Go cassette was amplified from pD11-KDBH with primers 244-4Go F2 Inf SpeI (SEQ ID NO:72) and 253-4Go R3 (SEQ ID NO:73), digested with SpeI and cloned by ligation into pBKMS 184 SpeI site.

### Example 1B. Construction of a Vector Containing the Lysine Biosynthesis Genes ask-asd-dapB-ddh and a FarI Homology Region for Targeted Integration

The 3′ FarI homology region from a NRRL B-11474 derived strain was amplified by PCR using primers FarI 3P InfusF (SEQ ID NO:30) and FarI 3P InfusR AvrII (SEQ ID NO:31). The purified PCR fragment was cloned by IN-FUSION® reaction (Clontech) into pBKMS digested with NdeI and PstI to generate pBKMS FarI 3p. Similarly, the 5′ FarI homology region from a NRRL B-11474-derived strain was PCR amplified with primers FarI 5p InfusF (SEQ ID NO:32) and FarI 5p InfusR (SEQ ID NO:33) and cloned into the XbaI/AvrII sites of pBKMS FarI 3p to generate pBKMS FarI.

The 5.4 kb ask-asd-ddh-dapB-orj2′ cassette (4Go) was digested from pFC3-ask-asd-dapB-ddh plasmid (U.S. Pat. No. 7,368,276) with PmeI and XmaI and ligated into pDElia11 (U.S. Pat. No. 6,927,046) linearized with HincII and XmaI. The resulting vector pD11-KBDH was digested with NruI and SwaI to eliminate a ddh-dapB 2.9-kb fragment to generate pD11KD. pD11KD was then digested with SmaI and a 1.36 kb PCR fragment amplified from pD11KDBH using primers 685 (SEQ ID NO:74) and 686 (SEQ ID NO:75) was inserted using IN-FUSION® (Clontech) to generate pD11KDH. A 986-bp dapB fragment was then amplified from pD11KDBH with primers 687 (SEQ ID NO:76) and 693 (SEQ ID NO:77) and cloned into the pD11KDH SbfI site by IN-FUSION® (Clontech) reaction. The KBDH fragment was excised from the resulting pD11KBDH R plasmid by restriction with SpeI and cloned into the AvrII site of pBKMS FarI to generate pBKMSFarI4GRA.

### Example 2. Allelic Replacement in C. glutamicum

This examples describes the methods used for allelic replacement in Example 4, below. Strains were cultured in Medium B (Table 5) or BHI broth (BD Biosciences) at 30° C. until OD 660 nm reached 0.5. Cells were harvested at 4° C. by centrifugation, washed twice in ice-cold deionized water, and resuspended in ice-cold 10% glycerol to generate electrocompetent C. glutamicum cells. Plasmid and cells were mixed together, transformed by electroporation, plated on BHI agar plates with 10 μg/ml kanamycin, and incubated until transformants (kanamycin resistant, sucrose sensitive) which have integrated the plasmid appeared. Transformants were further cultivated overnight at 30° C. in Medium B (Table 5) supplemented with an additional 5% sucrose (final concentration 10% sucrose) and plated on Medium B to select for strains that excised the plasmid through a second recombination event and thus were sucrose resistant and kanamycin sensitive. Clones were further screened by PCR and sequencing to verify the presence of the desired mutation.

TABLE 5
Media used in Examples 2 and 3.

| | Medium | Medium | Medium | Medium | Medium | Medium | |
|---|---|---|---|---|---|---|---|
| | B | C | D | E | F | G | units |
| Volume | 1000 | 1000 | 1000 | 1000 | 1000 | 1000 | ml |
| glucose | | | 30 | 75 | 35 | 50 | g |
| Sucrose | 50 | 50 | | | | | g |
| L-alanine | 0.5 | | | | | | mg |
| L-methionine | 0.5 | | | | | | mg |
| l-threonine | 0.25 | | | | | | mg |
| KH₂PO₄ | | | | | | 1 | g |
| K₂HPO₄ | 3 | 3 | | 0.75 | 1 | | g |
| Urea | 3 | 3 | | | | | g |
| MgSO₄*7H₂O | 0.5 | 0.5 | | 0.3 | 0.4 | 0.4 | g |
| yeast extract | | 35 | | | | | g |
| Polypeptone | 20 | | | | | | g |
| peptone (BBL) | | | | | | | |
| Beef Extract | 5 | | | | | | g |
| (BBL) | | | | | | | |
| d-Biotin | 0.756 | 0.756 | 3.024 | 0.225 | 0.3 | 0.3 | mg |
| Thiamine*HCl | 3 | 3 | 3 | | | | mg |
| Niacinamide | 0.125 | 0.125 | 0.125 | | | | g |
| pH (KOH) | 7.3 | 7.3 | | 7.4 | 7.4 | 7.3 | |
| MnSO₄*H₂O | | | 0.01 | 0.0075 | 0.01 | 0.01 | g |
| FeSO4*7H₂O | | | | | 0.1 | 0.01 | g |
| (NH₄)₂SO₄ | | | | 37.5 | 50 | 50 | g |
| CSL Roquette | | | 20 | 15 | 20 | 20 | g |
| Raffinae¹ | | | 5 | | | | g |
| CaCO₃ | | | 50 | 50 | 50 | 50 | g |

### Example 3. Genomic DNA Extraction

For the extraction of genomic DNA, the selected strains were grown overnight in Medium B (Table 5) at 30° C. Cultures were precipitated by centrifugation at 5000×g (4° C.) for 10 minutes. The pellets were suspended in 10 ml of a solution containing 25 mM Tris-HCl pH 8.0, 10 mM EDTA, 50 mM glucose, and 20 mg/ml lysozyme and incubated for 2 hours at 37° C. The incubation was extended for an additional 2 hours following addition of 1.3 ml 10% SDS, 67 μl 10 mg/ml RNAse A, and 167 μl of Proteinase K (20 mg/ml stock). Genomic DNA was further purified by phenol-chloroform extraction and precipitation by addition of two volumes of ice cold ethanol and 0.1 volume of 3M sodium acetate (pH 5.2). After incubation at −80° C. for 1 hour, the DNA pellet was separated by centrifugation at 14,000 rpm (4° C.) for 1 hour, washed with 70% ethanol, air dried, and dissolved in nuclease-free water.

### Example 4. Assembly of Insertion Fragments into a pK18mobsacB-Derived Vector and Transformation into C. glutamicum Strains

### Example 4A. Assembly of Plasmids for Integration and Allelic Replacement

All DNA fragments used in the generation of upstream or downstream homologous recombination regions, as well as inserts, were either amplified from purified genomic DNA extracted from strain NRRL B-11474 or from plasmids containing sequences derived from this strain as described in Example 2 using the polymerase chain reaction (PCR), or were chemically synthesized (DNA 2.0). A pZ vector derived from pK18mobsacB (Schaffer et al., Gene 145: 69-73, 1994; Accession FJ437239) containing an URA3 gene for selection in yeast was used as the vector backbone for introducing the changes into C. glutamicum. Homology arms and inserts were assembled into the vector backbone by homologous recombination in yeast (Ma et al., Gene (58): 201-16, 1987). Each DNA fragment contained a 50-bp overlap on each side to ensure correct assembly with its adjacent parts. For assembly, the linearized vector backbone, the two homology arms, and optionally the inserted DNA fragment were simultaneously transformed into Saccharomyces cerevisiae CEN.PK (Entian & Kotter, Methods in Microbiology 36: 629-66, 2007) and plated on synthetic complete agar plates without uracil (Sigma). The assembled plasmids were extracted from yeast using a ZYMOPREP™ I yeast plasmid miniprep kit (Zymo Research) and propagated in E. coli 10-Beta cells (New England Biolabs) with 50 μg/ml kanamycin selection before transformation into C. glutamicum as described in Example 2.

### Example 4B. Construction of Transformation Vector to Introduce rnaJ_{G448S} Allele

Two homology arms were amplified using PCR from NRRL B-11474 genomic DNA obtained as described in Example 3.

The approximately 2.1 kb downstream homology arm (nucleotides 2026 to 4101 from SEQ ID NO:1) was amplified using primers SNP_084_P1 (SEQ ID NO:38) and SNP_084_P2 (SEQ ID NO:39). Primer SNP_084_P1 includes a 50-nucleotide 5′ extension that overlaps with the pZ backbone described in Example 4A. Primer SNP_084_P2 contains a G to A substitution at position 25 to introduce the G448S substitution. Similarly, the approximately 2.1 kb upstream homology arm (nucleotides 1 to 2075 from SEQ ID NO:1) was amplified using primers SNP_084_P3 (SEQ ID NO:40) and SNP_084_P4 (SEQ ID NO:41). Primer SNP_084_P3 contains a C to T substitution at position 20 to introduce the G448S substitution. SNP_084_P4 includes a 50-base 5′ extension that overlaps with the pZ described in Example 4A. Alignment of the two homology arms results in a 50-bp overlap defined by primers SNP_084_P2 and SNP_084_P3. The two homology arms were then assembled into pZ as described in Example 4A and transformed into C. glutamicum NRRL B-11474 and its derived strains as described in Example 2. Resulting strains carry the altered rnaJ coding sequence (SEQ ID NO:2), encoding the rnaJ amino acid sequence with the G448S substitution (SEQ ID NO:3).

### Example 4C. Construction of Transformation Vector to Introduce accDA_{G310E} Allele

Two homology arms were amplified from NRRL B-11474 genomic DNA by PCR. The 2070 bp downstream homology arm (nucleotides 2032 to 4101 from SEQ ID NO:4) was amplified using primers SNP_033_P1 (SEQ ID NO:46) and SNP_033_P2 (SEQ ID NO:47). Primer SNP_033_P1 includes a 50-nucleotide 5′ extension that overlaps with the pZ vector backbone described in Example 4A. Primers SNP_033_P2 contains a G to A substitution at position 20 to introduce the G310E mutation. Similarly, the approximately 2.1 kb upstream homology arm (nucleotides 1 to 2071 from SEQ ID NO:4) was amplified using primers SNP_084_P3 (SEQ ID NO:48) and SNP_084_P4 (SEQ ID NO:49). Primer SNP_033_P3 contains a C to T substitution at position 21 to introduce the G310E mutation. SNP_033_P4 includes a 50-base 5′ extension that overlaps with the pZ backbone described in Example 4A. Alignment of the two homology arms results in a 40-bp overlap defined by primers SNP_033_P2 and SNP_033_P3. The two homology arms were then assembled into pZ as described in Example 4A and transformed into C. glutamicum NRRL B-11474 and its derived strains as described in Example 2. Resulting strains carry the altered accDA coding sequence (SEQ ID NO:5), encoding the accDA amino acid sequence with the G310E substitution (SEQ ID NO:6).

### Example 4D. Construction of Transformation Vector to Introduce cg1144_{P66S} Allele

Two homology arms were amplified from NRRL B-11474 genomic DNA by PCR. The 2037 bp upstream homology arm (nucleotides 1 to 2037 from SEQ ID NO:7) was amplified using primers SNP_316_P1 (SEQ ID NO:50) and SNP_316_P2 (SEQ ID NO:51). Primer SNP_316_P1 includes a 50-nucleotide 5′ extension that overlaps with the pZ vector backbone described in Example 4A. Primer SNP_316_P2 contains a G to A substitution at position 21 to introduce the P66S mutation. Similarly, the 2070 bp downstream homology arm (nucleotides 1998 to 4067 from SEQ ID NO:7) was amplified using primers SNP_316_P3 (SEQ ID NO:52) and SNP_316_P4 (SEQ ID NO:53). Primer SNP_316_P3 contains a C to T substitution at position 20 to introduce the P66S mutation. SNP_316_P4 includes a 50-base 5′ extension that overlaps with the pZ backbone described in Example 4A. Alignment of the two homology arms results in a 40-bp overlap defined by primers SNP_316_P2 and SNP_316_P3. The two homology arms were then assembled into pZ as described in Example 4A and transformed into C. glutamicum NRRL B-11474 and its derived strains as described in Example 2. Resulting strains carry the altered cg1144 coding sequence (SEQ ID NO:8), encoding the cg1144 amino acid sequence with the P66S substitution (SEQ ID NO:9).

### Example 4E. Construction of Transformation Vector to Replace the Native pyc Promoter by Promoter Pcg1860

Two homology arms were amplified from NRRL B-11474 genomic DNA by PCR. The 2043 bp upstream homology arm (nucleotides 1 to 2043 from SEQ ID NO:10) was amplified using primers Pcg1860_pyc_P1 (SEQ ID NO:42) and Pcg1860_pyc_P2 (SEQ ID NO:43). Similarly, the 2050 bp downstream homology arm (nucleotides 2161 to 4210 from SEQ ID NO:10) was amplified using primers Pcg1860_pyc_P3 (SEQ ID NO:44) and Pcg1860_pyc_P4 (SEQ ID NO:45). The 93-bp Pcg1860 nucleotide sequence (SEQ ID NO:11) was obtained by overlap of the 5′ extensions of primers Pcg1860_pyc_P2 and Pcg1860-pyc_P3. The two homology arms were then assembled into pZ as described in Example 4A and transformed into C. glutamicum NRRL B-11474 and its derived strains as described in Example 2. Resulting strains carry the promoter Pcg1860 (SEQ ID NO:11) instead of the pyc promoter (SEQ ID NO:12).

### Example 4F. Construction of Transformation Vector to Introduce aceE_{atg>ttg} Allele

Two homology arms were amplified from NRRL B-11474 genomic DNA by PCR. The 2072 bp upstream homology arm (nucleotides 1 to 2072 from SEQ ID NO:13) was amplified using primers SNP_aceE_P1 (SEQ ID NO:54) and SNP_aceE_P2 (SEQ ID NO:55). Primer SNP_aceE_P1 includes a 50-nucleotide 5′ extension that overlaps with the pZ vector backbone described in Example 4A. Primer SNP_aceE_P2 contains a T to A substitution at position 22 to introduce the A mutation. Similarly, the 2073 bp downstream homology arm (nucleotides 2029 to 4101 from SEQ ID NO:13) was amplified using primers SNP_aceE_P3 (SEQ ID NO:56) and SNP_aceE_P4 (SEQ ID NO:57). Primer SNP_aceE_P3 contains an A to T substitution at position 23 to introduce the T mutation. SNP_aceE_P4 includes a 50-base 5′ extension that overlaps with the pZ backbone described in Example 4. Alignment of the two homology arms results in a 44-bp overlap defined by primers SNP_aceE_P2 and SNP_aceE_P3. The two homology arms were then assembled into pZ as described in Example 4A and transformed into C. glutamicum NRRL B-11474 and its derived strains as described in Example 2. Resulting strains carry the TTG start codon in the aceE open reading frame (SEQ ID NO:14).

### Example 4G. Insertion of a Codon-Optimized lysA Coding Sequence

A cassette containing the codon optimized lysA fragment of SEQ ID NO:16 was inserted between nucleotides 2048 and 2049 of SEQ ID NO:15 and synthetically assembled (DNA 2.0) into the pZ vector described in Example 4A. The final plasmid contains two −2 kb homology arms (nucleotides 1 to 2048, and nucleotides 2049 to 4099 of SEQ ID NO:15) flanking a Pcg0007_39-lysA^{CO}-sodT (SEQ ID NO:17). The lysA^{CO} containing plasmid was transformed into C. glutamicum NRRL B-11474 and its derived strains as described in Example 2. Resulting strains carry an additional copy of the codon-optimized lysA coding sequence under the control of promoter Pcg0007_39.

### Example 4H. Insertion of a Promoter Upstream of the zwf Coding Sequence

Two homology arms were amplified from a NRRL B-11474 genomic DNA by PCR. The 2050 bp upstream homology arm (nucleotides 1 to 2050 from SEQ ID NO:19) was amplified using primers Pcg0007_39-zwf_P1 (SEQ ID NO:34) and Pcg0007_39-zwf_P2 (SEQ ID NO:35) Similarly, the 2050 bp downstream homology arm (nucleotides 2052 to 4101 from SEQ ID NO:19) was amplified using primers Pcg0007_39-zwf_P3 (SEQ ID NO:36) and Pcg0007_39-zwf_P4 (SEQ ID NO:37). The 93-bp Pcg0007_39 nucleotide sequence was obtained by overlap of the 5′ extensions of primers Pcg0007_39-zwf_P2 and Pcg0007_39-pyc_P3. The two homology arms were then assembled into pZ as described in Example 4A and transformed into C. glutamicum NRRL B-11474 and its derived strains as described in Example 2. Resulting strains result in the replacement of nucleotide 2051 of SEQ ID NO:19 by the promoter Pcg0007_39 (SEQ ID NO:20). Resulting strains carry the promoter Pcg0007_39 upstream of the zwf ORF (SEQ ID NO:21).

### Example 4I. Replacement of Native opcA Coding Sequence by the opcA Coding Sequence of C. humireducens NBRC 106098

Two homology arms were amplified from NRRL B-11474-derived strain BS2CZ genomic DNA (Example 3) by PCR. The 2039 bp upstream homology arm (nucleotides 1 to 2039 from SEQ ID NO:22) was amplified using primers opcA_P1 (SEQ ID NO:58) and opcA_P2 (SEQ ID NO:59). Similarly, the 2083 bp downstream homology arm (nucleotides 3000 to 5082 from SEQ ID NO:22) was amplified using primers opcA_P3 (SEQ ID NO:60) and opcA_P4 (SEQ ID NO:61). A gene cassette consisting of the 93-bp Pcg0007_39 nucleotide sequence (SEQ ID NO:20) and C. humireducens opcA (SEQ ID NO:23) carries sequence overlaps of the 5′ extensions of primers opcA_P2 and opcA_P3. The two homology arms and the gene cassette were then assembled into pZ as described in Example 4A and transformed into C. glutamicum NRRL B-11474 and its derived strains as described in Example 2. Resulting strains result in the replacement of nucleotide 2040 to 2999 of SEQ ID NO:22 by the Pcg0007_39-opcA (SEQ ID NO:24).

### Example 4J. Construction of Transformation Vector to Insert Promoter Pcg0007_39 in Front of the cg2766 Start Codon

Two homology arms were amplified from NRRL B-11474 genomic DNA by PCR. The 2050 bp downstream homology arm (nucleotides 2051 to 4100 from SEQ ID NO:26) was amplified using primers Pcg0007_39-cg2766_P1 (SEQ ID NO:62) and Pcg0007_39-cg2766_P2 (SEQ ID NO:63). Similarly, the 2050 bp upstream homology arm (nucleotides 1 to 2050 from SEQ ID NO:26) was amplified using primers Pcg0007_39-cg2766_P3 (SEQ ID NO:64) and Pcg0007_39-cg2766_P4 (SEQ ID NO:65). The 93-bp Pcg0007_39 nucleotide sequence (SEQ ID NO:20) was obtained by overlap of the 5′ extensions of primers Pcg0007_39-cg2766_P2 and Pcg0007_39-cg2766_P3. The two homology arms were then assembled into pZ as described in Example 4A and transformed into C. glutamicum NRRL B-11474 and its derived strains as described in Example 2. Resulting strains carry the promoter Pcg0007_39 inserted in front of cg2766 (SEQ ID NO:27).

### Example 4K. Construction of Transformation Vector to Replace the actA Promoter with Promoter Pcg0007_39

Two homology arms were amplified from NRRL B-11474 genomic DNA (Example 3) by PCR.

The 2021 bp downstream homology arm (nucleotides 2263 to 4283 from SEQ ID NO:28) was amplified using primers Pcg0007_39-actA_P1 (SEQ ID NO:66) and Pcg0007_39-actA_P2 (SEQ ID NO:67). Similarly, the 2050 bp upstream homology arm (nucleotides 1 to 2050 from SEQ ID NO:28) was amplified using primers Pcg0007_39-actA_P3 (SEQ ID NO:68) and Pcg0007_39-actA_P4 (SEQ ID NO:69). The 93-bp Pcg0007_39 nucleotide sequence (SEQ ID NO:20) was obtained by overlap of the 5′ extensions of primers Pcg0007_39-actA_P2 and Pcg0007_39-actA_P3. The two homology arms were then assembled into pZ as described in Example 4A and transformed into C. glutamicum NRRL B-11474 and its derived strains as described in Example 2. Resulting strains carry the promoter Pcg0007_39 in place of nucleotides 2051 to 2262 of SEQ ID NO:28 and inserted in front of actA (SEQ ID NO:29).

### Example 4L. Construction of Transformation Vector to Insert Promoter Pcg0007_39 in Front of the cg1383 Start Codon

Two homology arms were amplified from a NRRL B-11474 genomic DNA by PCR. The 2043 bp upstream homology arm (nucleotides 1 to 2043 of SEQ ID NO:98) was amplified using primers Pcg0007_39-cg1383_P1 (SEQ ID NO:99) and Pcg0007_39-cg1383_P2 (SEQ ID NO:100). Similarly, the 2050 bp downstream homology arm (nucleotides 2044 to 4093 of SEQ ID NO:98) was amplified using primers Pcg0007_39-cg1383_P3 (SEQ ID NO:101) and Pcg0007_39-cg1383_P4 (SEQ ID NO:102). The 93-bp Pcg0007_39 nucleotide sequence was obtained by overlap of the 5′ extensions of primers Pcg0007_39-cg1383_P2 and Pcg0007_39-cg1383_P3. The two homology arms were then assembled into pZ as described in Example 4A and transformed into C. glutamicum NRRL B-11474 and its derived strains as described in Example 2. Resulting strains carry the Pcg0007_39 promoter inserted in front of cg1383 start codon (SEQ ID NO:103).

### Example 4M. Construction of Transformation Vector to Replace the Native phoU Promoter with Promoter Pcg0007_39

Two homology arms were amplified from a NRRL B-11474 genomic DNA by PCR. The 1935 bp upstream homology arm (nucleotides 1 to 1935 of SEQ ID NO:104) was amplified using primers Pcg0007_39-phoU_P1 (SEQ ID NO:105) and Pcg0007_39-phoU_P2 (SEQ ID NO:106). Similarly, the 2050 bp downstream homology arm (nucleotides 1995 to 4044 of SEQ ID NO:104) was amplified using primers Pcg0007_39-phoU_P3 (SEQ ID NO:107) and Pcg0007_39-phoU_P4 (SEQ ID NO:108). The 93-bp Pcg0007_39 nucleotide sequence is obtained by overlap of the 5′ extensions of primers Pcg0007_39-phoU_P2 and Pcg0007_39-phoU_P3. The two homology arms are then assembled into pZ as described in Example 4A and transformed into C. glutamicum NRRL B-11474 and its derived strains as described in Example 2. Resulting strains carry the Pcg0007_39 promoter in front of phoU start codon (SEQ ID NO:109) and replacing nucleotides 1936-1994 of SEQ ID NO:104.

### Example 4N. Construction of Transformation Vector to Replace the Native Cg3210 Promoter with Promoter Pcg0007_39

Two homology arms were amplified from a NRRL B-11474 genomic DNA by PCR. The 1935 bp upstream homology arm (nucleotides 1 to 2030 of SEQ ID NO:110) were amplified using primers Pcg0007_39-cg3210_P1 (SEQ ID NO:111) and Pcg0007_39-cg3210_P2 (SEQ ID NO:112). Similarly, the 2047 bp downstream homology arm (nucleotides 2090 to 4136 of SEQ ID NO:110) were amplified using primers Pcg0007_39-cg3210_P3 (SEQ ID NO:113) and Pcg0007_39-3210_P4 (SEQ ID NO:114). The 93-bp Pcg0007_39 nucleotide sequence was obtained by overlap of the 5′ extensions of primers Pcg0007_39-cg3210_P2 and Pcg0007_39-cg3210_P3. The two homology arms are then assembled into pZ as described in Example 4A and transformed into C. glutamicum NRRL B-11474 and its derived strains as described in Example 2. Resulting strains carry the Pcg0007_39 replacing nucleotides 2031 to 2089 of SEQ ID NO:110, and inserted in front of cg3210 start codon (SEQ ID NO:115).

### Example 4O. Construction of Transformation Vector to Replace the Native Cg0800 Promoter with Promoter Pcg0007_39

Two homology arms were amplified from a NRRL B-11474 genomic DNA by PCR. The 2043 bp upstream homology arm (nucleotides 1 to 2043 of SEQ ID NO:116) was amplified using primers Pcg0007_39-cg0800_P3 (SEQ ID NO:117) and Pcg0007_39-cg0800_P4 (SEQ ID NO:118). Similarly, the 2050 bp downstream homology arm (nucleotides 2184 to 4233 of SEQ ID NO:116) was amplified using primers Pcg0007_39-cg0800_P1 (SEQ ID NO:119) and Pcg0007_39-0800_P2 (SEQ ID NO:120). The 93-bp Pcg0007_39 nucleotide sequence was obtained by overlap of the 5′ extensions of primers Pcg0007_39-cg0800_P2 and Pcg0007_39-cg0800_P3. The two homology arms were then assembled into pZ as described in Example 4A and transformed into C. glutamicum NRRL B-11474 and its derived strains as described in Example 2. Resulting strains carry the Pcg0007_39 promoter inserted in front of cg0800 start codon (SEQ ID NO:121), and replacing nucleotides 2044-2183 of SEQ ID NO:116.

### Example 5. Evaluation of Altered C. glutamicum Strains for Lysine Production in Microtiter Plates

### Example 5A. Lysine Production by Altered C. glutamicum Strains

Each of the altered strains described in the previous examples as well as strains resulting from combining the different alterations were evaluated for lysine production by a three-stage fermentation in microtiter plates. Cells were grown in Medium C (Table 5). After 48 hours a 10% inoculum was transferred to Medium D (Table 5). Ten percent of the cell culture was transferred after 19 hours to Medium E (Table 5) and grown for an additional 24 hours or 96 hours. Culture conditions were 32° C., 1000 rpm (Infors HT).

The amount of lysine produced was determined using a coupled lysine oxidase assay. Culture supernatants were added to a lysine oxidase reaction solution (250 mM potassium phosphate buffer, pH 7.5, 824 mg/ml phenol, 76 mg/ml 4-amino antipyrene (Sigma), 0.03 mg/ml peroxidase (Sigma), 0.015 units/ml Lysine oxidase (Sigma). Samples were incubated at 25° C. for 40 min and absorbance was read at 490 nm. The amount of dextrose in the microtiter plates was determined by a couple glucose oxidase assay. Culture supernatants were added to a glucose oxidase reaction solution (275 mM sodium maleate buffer pH 5.5, 730 mg/ml phenol, 680 mg/ml 4-amino antipyrene (Sigma), 0.027 mg/ml peroxidase (Sigma), 56 mg/L glucose oxidase (Sigma). Samples were incubated at 25° C. for 40 min and absorbance was read at 490 nm.

The amount of lysine (mM) produced by each recombinant strain and its immediate parent strain is shown in the Table 6. Table 7A is a list of genomic alterations, and Table 7B identifies which alterations are present in the tested strains with respect to the native genome of the bacterium deposited as NRRL B-11474.

TABLE 6

| Parent | Modified | Change from Parent to | Lysine production (mM) - 24 hrs | | Lysine production (mM) - 96 hrs | |
|---|---|---|---|---|---|---|
| Strain ID | Strain ID | Modified Strain | Parent Strain | Modified Strain | Parent Strain | Modified Strain |
| 34507 | 88274 | 0007_39-cg0800 | 5.51 +/− 3.24 | 4.84 +/− 1.85 | 47.62 +/− 6.82 | 54.13 +/− 1.02 |
| 121645 | 139804 | 0007_39-cg0800 | 20.22 +/− 4.62 | 14.59 +/− 1.54 | 49.84 +/− 4.51 | 53.08 +/− 1.41 |
| 151834 | 174547 | 0007_39-cg0800 | 5.33 +/− 9.59 | 12.71 +/− 10.45 | 57.98 +/− 4.13 | 56.78 +/− 4.55 |
| 174205 | 182533 | 0007_39-cg0800 | 12.82 +/− 7.93 | 17.86 +/− 2.46 | 55.51 +/− 5.11 | 61.54 +/− 2.09 |
| 174275 | 182550 | 0007_39-cg0800 | 18.06 +/− 5.8 | 20.24 +/− 5.46 | 58.89 +/− 4.31 | 57.48 +/− 3.8 |
| 174275 | 182557 | 0007_39-cg0800 | 18.06 +/− 5.8 | 19.3 +/− 2.73 | 58.89 +/− 4.31 | 63.14 +/− 2.55 |
| 175088 | 182566 | 0007_39-cg0800 | 19.65 +/− 5.47 | 19.83 +/− 5.4 | 59.7 +/− 3.89 | 57.61 +/− 3.73 |
| 175093 | 182574 | 0007_39-cg0800 | 26.29 +/− 8.54 | 21.54 +/− 6.77 | 55.33 +/− 6.84 | 60.49 +/− 2.8 |
| 175109 | 182591 | 0007_39-cg0800 | 24.07 +/− 6.91 | 16.86 +/− 6.38 | 53.94 +/− 3.57 | 56.96 +/− 2.37 |
| 175109 | 182598 | 0007_39-cg0800 | 24.07 +/− 6.91 | 33.71 +/− 5.63 | 53.94 +/− 3.57 | 58.56 +/− 2.41 |
| 175784 | 182601 | 0007_39-cg0800 | 26.06 +/− 11.12 | 17.64 +/− 6.7 | 57.43 +/− 2.77 | 59.8 +/− 2.57 |
| 175784 | 182608 | 0007_39-cg0800 | 26.06 +/− 11.12 | 25.56 +/− 2.25 | 57.43 +/− 2.77 | 61.07 +/− 2.22 |
| 176538 | 182609 | 0007_39-cg0800 | 25.6 +/− 6.2 | 28.81 +/− 2.81 | 57.82 +/− 5.05 | 62.76 +/− 1.23 |
| 176538 | 182616 | 0007_39-cg0800 | 25.6 +/− 6.2 | 24.35 +/− 2.7 | 57.82 +/− 5.05 | 64.08 +/− 1.6 |
| 176509 | 182617 | 0007_39-cg0800 | 23.21 +/− 11.77 | 9.03 +/− 3.72 | 62.92 +/− 4.61 | 66.83 +/− 3.8 |
| 176509 | 182624 | 0007_39-cg0800 | 23.21 +/− 11.77 | 15.26 +/− 2.45 | 62.92 +/− 4.61 | 66.74 +/− 1.18 |
| 174547 | 183652 | 0007_39-cg0800 | 12.71 +/− 10.45 | 20.12 +/− 6.82 | 56.78 +/− 4.55 | 61.19 +/− 1.97 |
| 175155 | 183691 | 0007_39-cg0800 | 21.4 +/− 5.14 | 21.27 +/− 2.26 | 56.03 +/− 3.66 | 52.58 +/− 2.62 |
| 175174 | 183700 | 0007_39-cg0800 | 23.97 +/− 5.96 | 24.65 +/− 5.13 | 57.11 +/− 2.8 | 55.95 +/− 4.59 |
| 175776 | 183708 | 0007_39-cg0800 | 30.83 +/− 10.18 | 20.58 +/− 4.21 | 58.82 +/− 4.55 | 55.64 +/− 4.54 |
| 175785 | 183715 | 0007_39-cg0800 | 26.69 +/− 6.46 | 26.39 +/− 2.62 | 57.18 +/− 3.68 | 53.79 +/− 2.97 |
| 175785 | 183716 | 0007_39-cg0800 | 26.69 +/− 6.46 | 23.11 +/− 5.68 | 57.18 +/− 3.68 | 51.52 +/− 2.25 |
| 178745 | 187964 | 0007_39-cg0800 | 18.28 +/− 9.23 | 8.24 +/− 1.95 | 60.96 +/− 4.7 | 63.09 +/− 1.94 |
| 180398 | 187972 | 0007_39-cg0800 | 23.43 +/− 12.62 | 10.26 +/− 1.19 | 57.63 +/− 3.9 | 59.75 +/− 1.86 |
| 180402 | 187984 | 0007_39-cg0800 | 22.94 +/− 15.61 | 13.82 +/− 2.75 | 52.86 +/− 5.07 | 57.71 +/− 2.64 |
| 178726 | 188102 | 0007_39-cg0800 | 15.7 +/− 12.23 | 5.54 +/− 3.79 | 62.47 +/− 3.79 | 60.2 +/− 1.47 |
| 178726 | 188104 | 0007_39-cg0800 | 15.7 +/− 12.23 | 4.62 +/− 4.5 | 62.47 +/− 3.79 | 61.32 +/− 1.82 |
| 180390 | 188154 | 0007_39-cg0800 | 26.07 +/− 11.03 | 19.31 +/− 8.45 | 57.26 +/− 2.63 | 58.87 +/− 2.1 |
| 176538 | 188162 | 0007_39-cg0800 | 25.6 +/− 6.2 | 22.58 +/− 2.65 | 57.82 +/− 5.05 | 61.01 +/− 1.67 |
| 176538 | 188169 | 0007_39-cg0800 | 25.6 +/− 6.2 | 14.06 +/− 4.59 | 57.82 +/− 5.05 | 61.24 +/− 1.71 |
| 175229 | 188170 | 0007_39-cg0800 | 14.97 +/− 7.27 | 15.19 +/− 3.38 | 42.82 +/− 18.51 | 64.79 +/− 1.95 |
| 181567 | 188178 | 0007_39-cg0800 | 16.82 +/− 11.51 | 4.96 +/− 3.88 | 58.84 +/− 5.97 | 61.7 +/− 3.84 |
| 180384 | 188186 | 0007_39-cg0800 | 27.09 +/− 13.8 | 23.88 +/− 12.17 | 56.9 +/− 3.43 | 60.93 +/− 2.15 |
| 182562 | 190037 | 0007_39-cg0800 | 15.13 +/− 6.02 | 18.61 +/− 1.81 | 61.26 +/− 3.72 | 60.35 +/− 1.59 |
| 182563 | 190045 | 0007_39-cg0800 | 23.62 +/− 7.18 | 19.63 +/− 1.79 | 62.13 +/− 3.81 | 60.13 +/− 1.86 |
| 182612 | 190053 | 0007_39-cg0800 | 21.67 +/− 4.04 | 17.96 +/− 6.16 | 62.94 +/− 4.44 | 58.86 +/− 2.58 |
| 182620 | 190061 | 0007_39-cg0800 | 19.34 +/− 6.03 | 18.17 +/− 5.41 | 61.92 +/− 4.76 | 63.53 +/− 3.46 |
| 182623 | 190069 | 0007_39-cg0800 | 23.86 +/− 4.55 | 25.51 +/− 7.13 | 58.63 +/− 5.35 | 59.76 +/− 3.09 |
| 179967 | 191527 | 0007_39-cg0800 | 9.16 +/− 5.93 | 9.28 +/− 4.58 | 61.7 +/− 5.41 | 58.22 +/− 4.37 |
| 182566 | 192419 | 0007_39-cg0800 | 19.83 +/− 5.4 | 22.34 +/− 3.99 | 57.61 +/− 3.73 | 53.9 +/− 2.05 |
| 176538 | 193279 | 0007_39-cg0800 | 25.6 +/− 6.2 | 26.4 +/− 2.41 | 57.82 +/− 5.05 | 56.47 +/− 4.58 |
| 182612 | 193295 | 0007_39-cg0800 | 21.67 +/− 4.04 | 24.95 +/− 5.13 | 62.94 +/− 4.44 | 56.12 +/− 5.52 |
| 191541 | 197907 | 0007_39-cg0800 | 13.96 +/− 7.55 | 19.08 +/− 3.27 | 63.49 +/− 3.11 | 62.35 +/− 2.05 |
| 191520 | 206822 | 0007_39-cg0800 | 14.26 +/− 5.09 | 14.33 +/− 2.29 | 64.17 +/− 3.8 | 61.85 +/− 1.8 |
| 193318 | 213002 | 0007_39-cg0800 | 24 +/− 6.35 | 21.79 +/− 3.62 | 61.14 +/− 3.26 | 57.62 +/− 1.17 |
| 193318 | 213012 | 0007_39-cg0800 | 24 +/− 6.35 | 24.84 +/− 3.03 | 61.14 +/− 3.26 | 61.88 +/− 4.03 |
| 00000 | 219284 | 0007_39-cg0800 | 5.06 +/− 5.44 | 3.82 +/− 3.9 | 24.04 +/− 7.01 | 36.76 +/− 4.58 |
| 121645 | 219308 | 0007_39-cg0800 | 20.22 +/− 4.62 | 20.06 +/− 8.89 | 49.84 +/− 4.51 | 52.49 +/− 4.39 |
| 34507 | 87642 | 0007_39-cg1383 | 5.51 +/− 3.24 | 10.65 +/− 2.82 | 47.62 +/− 6.82 | 48.74 +/− 4.66 |
| 81651 | 121624 | 0007_39-cg 1383 | 9.15 +/− 2.55 | 9.27 +/− 2.27 | 43.53 +/− 2.69 | 47.46 +/− 2.64 |
| 99349 | 121638 | 0007_39-cg1383 | 16.22 +/− 3.6 | 17.45 +/− 1.02 | 53.98 +/− 3.54 | 52.36 +/− 1 |
| 99370 | 121643 | 0007_39-cg 1383 | 13.58 +/− 3.8 | 11.72 +/− 2.78 | 51.8 +/− 4.46 | 49.6 +/− 1.97 |
| 71075 | 132568 | 0007_39-cg 1383 | 23.93 +/− 5.69 | 27.33 +/− 3.66 | 51.37 +/− 5.54 | 56.21 +/− 2.84 |
| 112373 | 134579 | 0007_39-cg 1383 | 19.39 +/− 4.03 | 17.46 +/− 1.34 | 52.21 +/− 4.81 | 51.73 +/− 1.92 |
| 121645 | 134603 | 0007_39-cg1383 | 20.22 +/− 4.62 | 18.52 +/− 3.41 | 49.84 +/− 4.51 | 52.89 +/− 2.6 |
| 108568 | 144042 | 0007_39-cg1383 | 26.79 +/− 7.43 | 14.54 +/− 8.65 | 52.58 +/− 5.38 | 60.47 +/− 5.14 |
| 108569 | 144051 | 0007_39-cg 1383 | 13.76 +/− 8.08 | 20.82 +/− 0.97 | 58.16 +/− 6.61 | 64.88 +/− 2.74 |
| 132598 | 148418 | 0007_39-cg1383 | 25.69 +/− 4.05 | 23.55 +/− 1.52 | 50.53 +/− 3.56 | 53.64 +/− 1.32 |
| 132581 | 148431 | 0007_39-cg1383 | 10.81 +/− 3.05 | 9.17 +/− 1.73 | 48.17 +/− 9.27 | 54.36 +/− 1.53 |
| 134595 | 148999 | 0007_39-cg1383 | 10.48 +/− 3.33 | 13.14 +/− 1.15 | 50.76 +/− 2.94 | 53 +/− 0.93 |
| 132599 | 149034 | 0007_39-cg1383 | 19.33 +/− 4.23 | 23.52 +/− 1.53 | 52.51 +/− 5.41 | 49.83 +/− 0.84 |
| 134610 | 149042 | 0007_39-cg1383 | 18.73 +/− 4.67 | 19.1 +/− 3.06 | 48.27 +/− 2.83 | 48.46 +/− 1.81 |
| 102381 | 149058 | 0007_39-cg1383 | 26.82 +/− 8.13 | 33.01 +/− 3.67 | 49.78 +/− 3.59 | 49.01 +/− 1.14 |
| 148401 | 151595 | 0007_39-cg1383 | 19.21 +/− 5.8 | 9.29 +/− 7.05 | 52.95 +/− 4.06 | 54.43 +/− 2.93 |
| 148401 | 151611 | 0007_39-cg 1383 | 19.21 +/− 5.8 | −11.43 +/− 1.47 | 52.95 +/− 4.06 | 0 +/− 0 |
| 148936 | 151753 | 0007_39-cg1383 | 20.42 +/− 10.42 | 29.69 +/− 4.69 | 44.38 +/− 7.76 | 52.61 +/− 7.57 |
| 148409 | 151764 | 0007_39-cg1383 | 11.55 +/− 4.46 | 10.69 +/− 2.77 | 56.02 +/− 2.26 | 56.33 +/− 1.12 |
| 148416 | 151804 | 0007_39-cg 1383 | 19.71 +/− 5.15 | 10.16 +/− 1.63 | 52.61 +/− 2 | 51.87 +/− 1.61 |
| 148989 | 151840 | 0007_39-cg 1383 | 22.37 +/− 3.54 | 23.75 +/− 3.91 | 41.08 +/− 3.55 | 40.95 +/− 3.54 |
| 149046 | 151904 | 0007_39-cg1383 | 18.43 +/− 5.34 | 19.02 +/− 3.73 | 52.81 +/− 5.31 | 50.01 +/− 4.06 |
| 149046 | 151914 | 0007_39-cg1383 | 18.43 +/− 5.34 | 17.83 +/− 3.84 | 52.81 +/− 5.31 | 46.8 +/− 0.96 |
| 148930 | 151920 | 0007_39-cg 1383 | 23.07 +/− 3.68 | 15.93 +/− 5.26 | 50.11 +/− 3.78 | 55.57 +/− 5.07 |
| 138706 | 151965 | 0007_39-cg1383 | 26.08 +/− 4.76 | 18.24 +/− 6.28 | 50.84 +/− 4.61 | 54.03 +/− 5.93 |
| 108568 | 152452 | 0007_39-cg 1383 | 26.79 +/− 7.43 | 25.7 +/− 1.89 | 52.58 +/− 5.38 | 54.45 +/− 0.96 |
| 102381 | 152468 | 0007_39-cg 1383 | 26.82 +/− 8.13 | 29.39 +/− 4.95 | 49.78 +/− 3.59 | 49.8 +/− 3.74 |
| 132590 | 152500 | 0007_39-cg 1383 | 26.43 +/− 7.04 | 24.96 +/− 2.09 | 43.76 +/− 5.2 | 44.2 +/− 0.9 |
| 151863 | 154589 | 0007_39-cg1383 | 17.14 +/− 4.77 | 22.8 +/− 1.45 | 55.32 +/− 4.32 | 52.28 +/− 2.72 |
| 151867 | 154600 | 0007_39-cg1383 | 19.54 +/− 4.74 | 18.29 +/− 5.44 | 48.04 +/− 5.55 | 49.91 +/− 5.85 |
| 151834 | 155462 | 0007_39-cg 1383 | 5.33 +/− 9.59 | 16.23 +/− 0.93 | 57.98 +/− 4.13 | 49.11 +/− 1.17 |
| 151834 | 155466 | 0007_39-cg 1383 | 5.33 +/− 9.59 | 15.89 +/− 1.64 | 57.98 +/− 4.13 | 49.56 +/− 0.95 |
| 151834 | 155469 | 0007_39-cg1383 | 5.33 +/− 9.59 | 17.14 +/− 1.1 | 57.98 +/− 4.13 | 49.3 +/− 1.13 |
| 151906 | 162735 | 0007_39-cg 1383 | 19.77 +/− 3.91 | 20.74 +/− 1.45 | 54.38 +/− 4.77 | 49.16 +/− 0.76 |
| 152616 | 163111 | 0007_39-cg 1383 | 12.28 +/− 4.92 | 10.65 +/− 2.71 | 55.25 +/− 2.57 | 53.29 +/− 1.51 |
| 152672 | 163167 | 0007_39-cg1383 | 22.62 +/− 6.35 | 16.67 +/− 2.9 | 53.52 +/− 4.63 | 48.45 +/− 1.79 |
| 154572 | 172144 | 0007_39-cg 1383 | 21.71 +/− 6.13 | 15.03 +/− 2.39 | 39.85 +/− 4.03 | 40.41 +/− 2.88 |
| 154591 | 172152 | 0007_39-cg 1383 | 17.14 +/− 5.9 | 12.96 +/− 1.6 | 50.61 +/− 3.77 | 52.72 +/− 2.16 |
| 154599 | 172168 | 0007_39-cg1383 | 20.27 +/− 4.57 | 15.68 +/− 3.72 | 50.86 +/− 3 | 51.08 +/− 1.63 |
| 155481 | 174249 | 0007_39-cg1383 | 16.87 +/− 6.35 | 14.66 +/− 13.4 | 55.17 +/− 4.66 | 56.8 +/− 4.07 |
| 162769 | 175204 | 0007_39-cg1383 | 9.47 +/− 2.63 | 9.35 +/− 0.75 | 51.09 +/− 4.23 | 57.99 +/− 1.77 |
| 155479 | 175787 | 0007_39-cg1383 | 25.76 +/− 13.89 | 14.66 +/− 7.05 | 54.62 +/− 4.15 | 55.9 +/− 2.03 |
| 158120 | 175821 | 0007_39-cg1383 | 24.09 +/− 11.01 | 14.42 +/− 6.62 | 55.15 +/− 5.86 | 53.72 +/− 1.55 |
| 155470 | 176476 | 0007_39-cg1383 | 19.35 +/− 8.82 | 25.29 +/− 6.91 | 54.58 +/− 4.66 | 59.02 +/− 3.23 |
| 155475 | 176484 | 0007_39-cg 1383 | 8.82 +/− 10.28 | 15.14 +/− 7.35 | 57.55 +/− 4.53 | 55.73 +/− 1.1 |
| 155475 | 176485 | 0007_39-cg 1383 | 8.82 +/− 10.28 | 13.75 +/− 8.3 | 57.55 +/− 4.53 | 55.32 +/− 1.25 |
| 163159 | 176545 | 0007_39-cg 1383 | 11.22 +/− 9.89 | 8.07 +/− 6.14 | 56.13 +/− 2.65 | 57.41 +/− 1.5 |
| 163159 | 176546 | 0007_39-cg1383 | 11.22 +/− 9.89 | 7.11 +/− 4.97 | 56.13 +/− 2.65 | 57.38 +/− 1.61 |
| 174232 | 180360 | 0007_39-cg1383 | 15.11 +/− 8.67 | 0.76 +/− 6.4 | 55.05 +/− 5.29 | 57.54 +/− 3.5 |
| 174547 | 183641 | 0007_39-cg1383 | 12.71 +/− 10.45 | 27.95 +/− 4.06 | 56.78 +/− 4.55 | 58.42 +/− 1.32 |
| 182550 | 193331 | 0007_39-cg 1383 | 20.24 +/− 5.46 | 24.15 +/− 2.8 | 57.48 +/− 3.8 | 58.09 +/− 1.39 |
| 193318 | 210851 | 0007_39-cg 1383 | 24 +/− 6.35 | 20.06 +/− 2.12 | 61.14 +/− 3.26 | 62.29 +/− 3.93 |
| 71075 | 101738 | 0007_39-cg3210 | 23.93 +/− 5.69 | 18.26 +/− 0.81 | 51.37 +/− 5.54 | 55.17 +/− 2.13 |
| 121645 | 139570 | 0007_39-cg3210 | 20.22 +/− 4.62 | 21.73 +/− 2.3 | 49.84 +/− 4.51 | 47.31 +/− 1.93 |
| 151834 | 174360 | 0007_39-cg3210 | 5.33 +/− 9.59 | 15.7 +/− 5.87 | 57.98 +/− 4.13 | 59.32 +/− 5.04 |
| 212960 | 279235 | 0007_39-cg3210 | 19.01 +/− 7.66 | 8.94 +/− 4.87 | 65.29 +/− 2.97 | 66.82 +/− 2.72 |
| 242504 | 279255 | 0007_39-cg3210 | 8.19 +/− 8.46 | 18.38 +/− 6.79 | 46.34 +/− 21.04 | 68.67 +/− 2.51 |
| 224392 | 279327 | 0007_39-cg3210 | 17.04 +/− 7.51 | 15.54 +/− 9.31 | 62.15 +/− 4.39 | 63.25 +/− 2.23 |
| 212953 | 279352 | 0007_39-cg3210 | 22.18 +/− 7.38 | 16.91 +/− 9.64 | 66.66 +/− 4.4 | 66.6 +/− 1.73 |
| 176509 | 182623 | 0007_39-cg3210 | 23.21 +/− 11.77 | 23.86 +/− 4.55 | 62.92 +/− 4.61 | 58.63 +/− 5.35 |
| 148936 | 151762 | 0007_39-phoU | 20.42 +/− 10.42 | 26.53 +/− 2.19 | 44.38 +/− 7.76 | 56.79 +/− 3.2 |
| 148409 | 151773 | 0007_39-phoU | 11.55 +/− 4.46 | 10.75 +/− 1.81 | 56.02 +/− 2.26 | 59.85 +/− 1.48 |
| 148401 | 151832 | 0007_39-phoU | 19.21 +/− 5.8 | 14.31 +/− 10.23 | 52.95 +/− 4.06 | 56.79 +/− 1.82 |
| 149046 | 151913 | 0007_39-phoU | 18.43 +/− 5.34 | 18.56 +/− 4.04 | 52.81 +/− 5.31 | 48.29 +/− 3.71 |
| 148930 | 151929 | 0007_39-phoU | 23.07 +/− 3.68 | 16.8 +/− 3.51 | 50.11 +/− 3.78 | 47.53 +/− 2.28 |
| 138706 | 151969 | 0007_39-phoU | 26.08 +/− 4.76 | 23.62 +/− 3.26 | 50.84 +/− 4.61 | 48.12 +/− 1.63 |
| 149004 | 151978 | 0007_39-phoU | 14.69 +/− 3.98 | 12.23 +/− 2.18 | 50.89 +/− 2.53 | 47.9 +/− 0.94 |
| 132590 | 152448 | 0007_39-phoU | 26.43 +/− 7.04 | 25.78 +/− 5.19 | 43.76 +/− 5.2 | 34.64 +/− 11.84 |
| 108568 | 152464 | 0007_39-phoU | 26.79 +/− 7.43 | 21.13 +/− 4.3 | 52.58 +/− 5.38 | 49.87 +/− 3.36 |
| 102381 | 152480 | 0007_39-phoU | 26.82 +/− 8.13 | 21.83 +/− 3 | 49.78 +/− 3.59 | 46.3 +/− 2.46 |
| 134595 | 152496 | 0007_39-phoU | 10.48 +/− 3.33 | 8.37 +/− 6.33 | 50.76 +/− 2.94 | 53.53 +/− 2.61 |
| 151790 | 152592 | 0007_39-phoU | 6.73 +/− 3.54 | 6.04 +/− 2.46 | 38.81 +/− 6.33 | 39.33 +/− 5.41 |
| 151772 | 152616 | 0007_39-phoU | 15.86 +/− 5.08 | 12.28 +/− 4.92 | 54.33 +/− 2.55 | 55.25 +/− 2.57 |
| 151778 | 152628 | 0007_39-phoU | 17.31 +/− 1.59 | 16.09 +/− 1.57 | 47.45 +/− 2.14 | 49.53 +/− 2.67 |
| 151605 | 152636 | 0007_39-phoU | 18.94 +/− 5.76 | 19.7 +/− 4.58 | 54.45 +/− 3 | 55.89 +/− 2.5 |
| 151834 | 152648 | 0007_39-phoU | 5.33 +/− 9.59 | 23 +/− 3.03 | 57.98 +/− 4.13 | 52.13 +/− 1.52 |
| 151739 | 152672 | 0007_39-phoU | 26.26 +/− 4.46 | 22.62 +/− 6.35 | 45.11 +/− 2.72 | 53.52 +/− 4.63 |
| 151920 | 155490 | 0007_39-phoU | 15.93 +/− 5.26 | 17.69 +/− 4.34 | 55.57 +/− 5.07 | 48.22 +/− 2.57 |
| 151920 | 155510 | 0007_39-phoU | 15.93 +/− 5.26 | 17.55 +/− 2.09 | 55.57 +/− 5.07 | 47.51 +/− 2.64 |
| 151904 | 155525 | 0007_39-phoU | 19.02 +/− 3.73 | 18.57 +/− 1.27 | 50.01 +/− 4.06 | 50.17 +/− 2.74 |
| 151965 | 155545 | 0007_39-phoU | 18.24 +/− 6.28 | 21.53 +/− 5.21 | 54.03 +/− 5.93 | 54.69 +/− 3.12 |
| 151863 | 162721 | 0007_39-phoU | 17.14 +/− 4.77 | 22.19 +/− 3.3 | 55.32 +/− 4.32 | 0 +/− 0 |
| 151906 | 162743 | 0007_39-phoU | 19.77 +/− 3.91 | 21.93 +/− 1.14 | 54.38 +/− 4.77 | 49.46 +/− 1.14 |
| 152645 | 162789 | 0007_39-phoU | 25.2 +/− 7.97 | 16.31 +/− 1.1 | 51.26 +/− 2.62 | 46.29 +/− 2.06 |
| 152645 | 163154 | 0007_39-phoU | 25.2 +/− 7.97 | 0 +/− 0 | 51.26 +/− 2.62 | 48.62 +/− 1.16 |
| 154572 | 172143 | 0007_39-phoU | 21.71 +/− 6.13 | 17.81 +/− 2.22 | 39.85 +/− 4.03 | 42.4 +/− 2.67 |
| 154591 | 172151 | 0007_39-phoU | 17.14 +/− 5.9 | 14.07 +/− 1.89 | 50.61 +/− 3.77 | 50.45 +/− 2.24 |
| 154650 | 172199 | 0007_39-phoU | 26.32 +/− 3.56 | 0 +/− 0 | 50.5 +/− 2.27 | 50.32 +/− 1.7 |
| 154660 | 172228 | 0007_39-phoU | 22.3 +/− 7.62 | 23.55 +/− 1.24 | 60.29 +/− 4.52 | 53.48 +/− 3.04 |
| 155464 | 174199 | 0007_39-phoU | 16.27 +/− 5.92 | 0 +/− 0 | 50.22 +/− 2.48 | 52.48 +/− 1.7 |
| 155475 | 174232 | 0007_39-phoU | 8.82 +/− 10.28 | 15.11 +/− 8.67 | 57.55 +/− 4.53 | 55.05 +/− 5.29 |
| 155479 | 174240 | 0007_39-phoU | 25.76 +/− 13.89 | 0 +/− 0 | 54.62 +/− 4.15 | 53.38 +/− 2.96 |
| 155481 | 174248 | 0007_39-phoU | 16.87 +/− 6.35 | 0 +/− 0 | 55.17 +/− 4.66 | 53.97 +/− 1.38 |
| 155554 | 174264 | 0007_39-phoU | 23.09 +/− 7.12 | 17.48 +/− 3.64 | 54.58 +/− 4.71 | 52.23 +/− 1.66 |
| 159431 | 174281 | 0007_39-phoU | 22.48 +/− 6.07 | 19.72 +/− 1.98 | 51.6 +/− 3.61 | 47.51 +/− 2.1 |
| 155470 | 175029 | 0007_39-phoU | 19.35 +/− 8.82 | 12.01 +/− 6.57 | 54.58 +/− 4.66 | 50.94 +/− 3.23 |
| 155475 | 175046 | 0007_39-phoU | 8.82 +/− 10.28 | 0 +/−0 | 57.55 +/− 4.53 | 51.7 +/− 2.26 |
| 155479 | 175062 | 0007_39-phoU | 25.76 +/− 13.89 | 0 +/−0 | 54.62 +/− 4.15 | 53.76 +/− 1.99 |
| 155554 | 175086 | 0007_39-phoU | 23.09 +/− 7.12 | 15.63 +/− 2.23 | 54.58 +/− 4.71 | 52.66 +/− 1.56 |
| 159431 | 175102 | 0007_39-phoU | 22.48 +/− 6.07 | 17.03 +/− 2.67 | 51.6 +/− 3.61 | 50.82 +/− 1.13 |
| 161403 | 175165 | 0007_39-phoU | 19.67 +/− 3.35 | 21.39 +/− 3.92 | 52.14 +/− 2.96 | 57.3 +/− 2.23 |
| 162718 | 175181 | 0007_39-phoU | 20.15 +/− 3.26 | 17.88 +/− 0.5 | 55.8 +/− 3.85 | 58.37 +/− 1.64 |
| 162753 | 175183 | 0007_39-phoU | 14.14 +/− 3.34 | 16.72 +/− 3.58 | 52.11 +/− 5.03 | 58.46 +/− 1.78 |
| 162753 | 175184 | 0007_39-phoU | 14.14 +/− 3.34 | 17.1 +/− 3.23 | 52.11 +/− 5.03 | 55.09 +/− 1.33 |
| 162769 | 175200 | 0007_39-phoU | 9.47 +/− 2.63 | 12.16 +/− 1.03 | 51.09 +/− 4.23 | 57.07 +/− 1.7 |
| 158120 | 175816 | 0007_39-phoU | 24.09 +/− 11.01 | 13.14 +/− 7.43 | 55.15 +/− 5.86 | 52.89 +/− 1.62 |
| 155554 | 176509 | 0007_39-phoU | 23.09 +/− 7.12 | 23.21 +/− 11.77 | 54.58 +/− 4.71 | 62.92 +/− 4.61 |
| 163159 | 176542 | 0007_39-phoU | 11.22 +/− 9.89 | 10.93 +/− 9.9 | 56.13 +/− 2.65 | 59.53 +/− 1.93 |
| 174275 | 183629 | 0007_39-phoU | 18.06 +/− 5.8 | 18.36 +/− 1.97 | 58.89 +/− 4.31 | 59.27 +/− 5.68 |
| 174547 | 186920 | 0007_39-phoU | 12.71 +/− 10.45 | 18.02 +/− 6.25 | 56.78 +/− 4.55 | 54.57 +/− 2.8 |
| 182566 | 187910 | 0007_39-phoU | 19.83 +/− 5.4 | 10.81 +/− 2.17 | 57.61 +/− 3.73 | 64.1 +/− 2.74 |
| 182574 | 187918 | 0007_39-phoU | 21.54 +/− 6.77 | 15.3 +/− 3.93 | 60.49 +/− 2.8 | 60.85 +/− 2.78 |
| 182591 | 187926 | 0007_39-phoU | 16.86 +/− 6.38 | 14.86 +/− 4.26 | 56.96 +/− 2.37 | 58.1 +/− 2.44 |
| 182601 | 187934 | 0007_39-phoU | 17.64 +/− 6.7 | 6.77 +/− 4.44 | 59.8 +/− 2.57 | 61.9 +/− 1.68 |
| 182617 | 187942 | 0007_39-phoU | 9.03 +/− 3.72 | 7.02 +/− 1.84 | 66.83 +/− 3.8 | 65.52 +/− 2.46 |
| 182550 | 187990 | 0007_39-phoU | 20.24 +/− 5.46 | 10.05 +/− 1.68 | 57.48 +/− 3.8 | 65.34 +/− 1.65 |
| 175776 | 188149 | 0007_39-phoU | 30.83 +/− 10.18 | 20.56 +/− 2.06 | 58.82 +/− 4.55 | 65.39 +/− 1.41 |
| 180390 | 188159 | 0007_39-phoU | 26.07 +/− 11.03 | 19.93 +/− 2.96 | 57.26 +/− 2.63 | 57.94 +/− 1.42 |
| 183661 | 190101 | 0007_39-phoU | 25 +/− 6.72 | 17.15 +/− 5.25 | 55.74 +/− 3.95 | 58.42 +/− 3.41 |
| 175776 | 193273 | 0007_39-phoU | 30.83 +/− 10.18 | 19.93 +/− 3.32 | 58.82 +/− 4.55 | 61.28 +/− 4.94 |
| 182566 | 193289 | 0007_39-phoU | 19.83 +/− 5.4 | 25.28 +/− 3.47 | 57.61 +/− 3.73 | 56.86 +/− 3.79 |
| 182566 | 193293 | 0007_39-phoU | 19.83 +/− 5.4 | 22.06 +/− 3.24 | 57.61 +/− 3.73 | 59.08 +/− 4.96 |
| 183175 | 196697 | 0007_39-phoU | 25.54 +/− 6.32 | 25.38 +/− 2.97 | 59.81 +/− 3.17 | 58.08 +/− 1.95 |
| 187911 | 197877 | 0007_39-phoU | 23.32 +/− 7.65 | 23.58 +/− 2.87 | 59.21 +/− 2.88 | 60.42 +/− 1.91 |
| 187929 | 197885 | 0007_39-phoU | 6.36 +/− 5.48 | 4.56 +/− 4.95 | 60.03 +/− 3.8 | 63.01 +/− 3.09 |
| 191496 | 197954 | 0007_39-phoU | 23.88 +/− 6.7 | 24.32 +/− 2.9 | 56.21 +/− 3.28 | 55.7 +/− 2.66 |
| 188141 | 201612 | 0007_39-phoU | 20.98 +/− 6.13 | 27.79 +/− 8.07 | 57.51 +/− 4.3 | 56.12 +/− 1.65 |
| 191500 | 201628 | 0007_39-phoU | 27.42 +/− 5.91 | 19.4 +/− 4.61 | 52.16 +/− 3.99 | 51.01 +/− 2.82 |
| 191504 | 201644 | 0007_39-phoU | 17.11 +/− 5.12 | 7.49 +/− 1.61 | 58.22 +/− 4.58 | 61.16 +/− 2.28 |
| 191520 | 201660 | 0007_39-phoU | 14.26 +/− 5.09 | 14.07 +/− 4.87 | 64.17 +/− 3.8 | 62.95 +/− 2.53 |
| 190123 | 201676 | 0007_39-phoU | 13.81 +/− 3.49 | 9.42 +/− 7.47 | 63.37 +/− 3 | 62.34 +/− 1.92 |
| 191520 | 206820 | 0007_39-phoU | 14.26 +/− 5.09 | 13.92 +/− 1.51 | 64.17 +/− 3.8 | 62.34 +/− 3.9 |
| 191500 | 206860 | 0007_39-phoU | 27.42 +/− 5.91 | 28.84 +/− 2.6 | 52.16 +/− 3.99 | 53.4 +/− 1.89 |
| 199384 | 212930 | 0007_39-phoU | 15.95 +/− 5.44 | 12.93 +/− 0.97 | 64.3 +/− 3.56 | 63.47 +/− 1.84 |
| 190061 | 212954 | 0007_39-phoU | 18.17 +/− 5.41 | 17.23 +/− 2.08 | 63.53 +/− 3.46 | 61.59 +/− 2.24 |

TABLE 7A

| Abbreviation | Description |
|---|---|
| 4GoB | insertion of lysine biosynthesis genes ask, asd, dapB, and ddh in the bioD |
| | region (Example 1) |
| 4GR | insertion of lysine biosynthesis genes ask, asd, dapB, and ddh in a FarI |
| | homology region (Example 1) |
| 0007_39-zwf | insertion of promoter Pcg0007_39 in front of the zwf open reading frame |
| DSS_084 | replacement of native maJ coding sequence with maJG448S allele |
| 1860-pyc | replacement of native pyc promoter with promoter Pcg1860 |
| DSS_033 | replacement of native accDA coding sequence with accDAG310E allele |
| DSS_316 | replacement of native cg1144 coding sequence with cg1144P66S allele |
| aceE_ATG > TTG | replacement of aceE start codon with TTG |
| lisa68 | insertion of codon-optimized lysA coding sequence under the control of |
| | promoter Pcg0007_39 and including sod terminator (SEQ ID NO: 17) |
| 0007_39_opca_P4 | replacement of native opcA coding sequence with opcA coding sequence |
| | for C. humireducens under the control of promoter Pcg0007_39 |
| 0007_39-actA | replacement of native actA promoter with promoter Pcg0007_39 |
| 0007_39-cg1383 | insertion of promoter Pcg0007_39 in front of cg1383 start codon |
| 0007_39-cg2766 | insertion of promoter Pcg0007_39 in front of cg2766 start codon |
| 0007_39-phoU | replacement of native phoU promoter with promoter Pcg0007_39 |
| 0007_39-cg3210 | replacement of native cg3210 promoter with promoter Pcg0007_39 |
| 0007_39-cg0800 | replacement of native cg0800 promoter with promoter Pcg0007_39 |
| Vhb04 | nitric oxide dioxygenase [Fischerella thermalis] |
| | WP_009454354.1 under the control of Pcg0007_39 |
| fumC_atg > gtg | replacement of fumC start codon with GTG |
| 0007_39-lysA | insertion of codon-optimized lysA sequence under the control of promoter |
| | Pcg0007_39 and having a sod terminator |
| 0007_39-CAA | insertion of codon-optimized glcp sequence (accession CAA34119.1) under |
| | the control of promoter Pcg0007_39 and codon optimized ppgK sequence |
| | (accession cg2091) under the control of Pcg1860 |
| 1860-xerD | replacement of native xerD promoter with promoter Pcg1860 |
| 0007_39-lpd | replacement of native lpd promoter with promoter Pcg0007_39 |
| 1860-aceF | replacement of native aceF promoter by promoter Pcg1860 |
| 0007_39-cg0725 | replacement of native cg0725 promoter with promoter Pcg0007_39 |
| DSS_121 | replacement of native cg3092 coding sequence with cg3092D213N allele |
| DSS_422 | replacement of native cg3180 coding sequence with cg3180W297* allele |
| 0007_39-SCO | insertion of codon-optimized SCO5578 sequence (accession NP_629713.1) |
| | under the control of promoter Pcg0007_39 and codon optimized ppgK |
| | sequence (accession cg2091) under the control of Pcg1860 |
| 1860-cg1768 | replacement of native cg1768 promoter by promoter Pcg1860 |
| 0007_39-rpoB | replacement of native rpoB promoter with promoter Pcg0007_39 |
| 0007_39-rpoB | same as 0007_39-rpoB |
| gene-383 | |
| 0007_39BL1631 | insertion of codon-optimized BL1631 sequence (accession NP_696783.1) |
| | under the control of promoter Pcg0007_39 and codon optimized ppgK |
| | sequence (accession cg2091) under the control of Pcg1860 |
| 0007_39-glf | replacement of native glf promoter with promoter Pcg0007_39 |
| glk | insertion of codon-optimized glf sequence (accession AAA27691.1) under |
| | the control of promoter Pcg0007_39 and codon optimized glk sequence |
| | (accession AHJ72364.1) under the control of Pcg1860 (at Ni487) |
| glk_2966 | same construct as above integrated at different insertion site (Ni2966) |
| 0007_39-NCgl1262 | replacement of native NCgl1262 promoter with promoter Pcg0007_39 |
| Pcg0007-amtB | replacement of native amtB promoter with Pcg0007 |
| Pcg0007_39-cg800 | replacement of native cg0800 promoter with Pcg0007_39 |
| 3121-mutM2 | replacement of native mutM promoter with promoter Pcg3121 |
| 1860-cg1144 | replacement of native cg1144 promoter with promoter Pcg1860 |
| 3121-gltD | replacement of native gltD promoter with promoter Pcg3121 |
| SCO_Ni2966 | same construct as 0007_39-SCO, integrated at Ni2966 |
| 1860-tkt | replacement of native tkt promoter with promoter Pcg1860 |
| 3381-cg1410 | replacement of native cg1410 promoter by promoter Pcg3381 (WO |
| | 2017/100376) |
| Pcg3121-prpB2 | replacement of the native promoter of cg0760 with Pcg0760 |
| 0007_39-nusG | replacement of native nusG promoter with promoter Pcg0007_39 |
| 0007_39-aroP | replacement of native aroP promoter with promoter Pcg0007_39 |
| 0007_39-rho | replacement of native rho promoter with promoter Pcg0007_39 |
| 0007_39-cg1503 | replacement of native cg1503 promoter with promoter Pcg0007_39 |
| 0007-39_ureR | replacement of native ureR promoter with promoter Pcg0007_39 |
| 0007_39-kgd | replacement of native kgd promoter with promoter Pcg0007_39 |
| 0007_39-cg2466 | replacement of native cg2466 promoter with promoter Pcg0007_39 |
| 0007_39-cg2899 | replacement of native cg2899 promoter with promoter Pcg0007_39 |
| 3121-cg1081 | replacement of native cg1081 promoter with promoter Pcg3121 |
| 0007_39-cg1486 | replacement of native cg1486 promoter with promoter Pcg0007_39 |
| 0007_39-Ncgl0304 | replacement of native Ncg10304 promoter with promoter Pcg0007_39 |
| 0007_39-Ncgl0304 | replacement of native Ncgl0304 promoter with promoter Pcg0007_39 |
| 0007_39-ncgl1511 | replacement of native Ncgl1511 promoter with promoter Pcg0007_39 |
| 0007_39-Ncgl0767 | replacement of native Ncgl0767 promoter with promoter Pcg0007_39 |
| 0755-dapA | replacement of native dapA promoter with promoter Pcg0755 |
| 0007_39-glpX | replacement of native glpX promoter with promoter Pcg0007_39 |

TABLE 7B
Genotypes of Modified Strains

| Modified | |
|---|---|
| Strain | Changes with respect to genome of NRRL B-11474 |
| 88274 | 0007_39-zwf; DSS_084; 1860-pyc; DSS_033; fumC_atg > gtg; 0007_39-opca_P4; 0007_39- |
| | cg0800 |
| 139804 | 0007_39-zwf; DSS_084; 1860-pyc; DSS_033; DSS_316; 0007_39-lysA; 0007_39-opcA_P4; |
| | 0007_39-cg0800 |
| 174547 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg0800 |
| 182533 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg0725; 0007-_39-rho; 0007_39-cg0800 |
| 182550 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800 |
| 182557 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800 |
| 182566 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800 |
| 182574 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-kgd; 0007_39-cg0800 |
| 182591 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-kgd; 0007_39-cg0800 |
| 182598 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-kgd; 0007_39-cg0800 |
| 182601 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 3121-cg1081; 0007_39-cg0800 |
| 182608 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 3121-cg1081; 0007_39-cg0800 |
| 182609 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg0800 |
| 182616 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg0800 |
| 182617 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU;0007_39-cg0800 |
| 182624 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-cg0800 |
| 183652 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg800; Pcg0007_39-cg800 |
| 183691 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-phoU; 0007_39-kgd; 0007_39-cg0800 |
| 183700 | 0007_39-zwf; DSS_084; 1860-pyc; DSS_033; DSS_316; 0007_39-lysA; 0007_39-lpd; |
| | opcA_P4; 1860-cg1768; glk; 0007_39-cg0725; 0007_39-cg2766; 0007_39-kgd; 0007_39- |
| | cg0800 |
| 183708 | 0007_39-zwf; DSS_084; 1860-pyc; DSS_033; DSS_316; 0007_39-lysA; 0007_39-lpd; |
| | opcA_P4; 0007_39-glf; 0007_39-cg1383; 0007_39-aroP; 0007_39-kgd; 0007_39-cg0800 |
| 183715 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-cg2466; 0007_39-cg0800 |
| 183716 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-cg2466; 0007_39-cg0800 |
| 187964 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-nusG; 0007_39- |
| | cg0800 |
| 187972 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-kgd; 0007_39-cg1503; 0007_39- |
| | cg0800 |
| 187984 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-kgd; 1860-xerD; 0007_39-cg0800 |
| 188102 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-mutM2; 0007_39-aroP; 0007_39-ureR; 0007_39- |
| | cg0800 |
| 188104 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-mutM2; 0007_39-aroP; 0007_39-ureR; 0007_39- |
| | cg0800 |
| 188154 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-kgd; 0007_39- |
| | cg0800 |
| 188162 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg0800 |
| 188169 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg0800 |
| 188170 | 0007_39-zwf; DSS_084; 1860-pyc; DSS_033; DSS_316; 0007_39-lysA; opcA_P4; lisa_68; |
| | 0007_39-rpoB; 0007_39-cg2766; 0007_39-aroP; 0007_39-cg0800 |
| 188178 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg2899; |
| | 0007_39-cg0800 |
| 188186 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-cg2899; 0007_39-kgd; 0007_39- |
| | cg0800 |
| 190037 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg2899; |
| | 0007_39-cg0800 |
| 190045 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-ureR; 0007_39- |
| | cg0800 |
| 190053 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg1486; 0007_39-cg0800 |
| 190061 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-cg1486; |
| | 0007_39-cg0800 |
| 190069 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-cg3210; |
| | 0007_39-cg0800 |
| 191527 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2960007_39-NCgl1262; 0007_39-cg0800 |
| 192419 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg1383; 0007_39-rpob; 0007_39-cg0800 |
| 193279 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg0800 |
| 193295 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg1486; 0007_39-cg0800 |
| 197907 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-cg1486; |
| | 0007_39-Ncgl0304; 0007_39-cg0800 |
| 206822 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-NCgl1262; 0007_39-cg0800 |
| 213002 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg1486; 1860-xerD; 0007_39-cg0800 |
| 213012 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg1486; 1860-xerD; 0007_39-cg0800 |
| 219284 | 0007_39-cg0800 |
| 219308 | 0007_39-zwf; DSS_084; 1860-pyc; DSS_033; DSS_316; 0007_39-lysA; opcA_P4; 0007_39- |
| | cg0800 |
| 87642 | 0007_39-zwf; DSS_084; 1860-pyc; DSS_033; fumC_atg > gtg; 0007_39-opca_P4; 0007_39- |
| | cg1383 |
| 121624 | 0007_39-zwf; DSS_084; 1860-pyc; DSS_033; fumC_ATG > TTG; opcA_P_4; Vhb04; 0007_39- |
| | cg1383 |
| 121638 | 0007_39-zwf; DSS_084; 1860-pyc; DSS_033; DSS_316; 0007_39-lysA; 0007_39-lpd; |
| | 0007_39-cg1383 |
| 121643 | 0007_39-zwf; DSS_084; 1860-pyc; DSS_033; fumC_ATG > GTG; opcA_P_4; 1860-aceF; |
| | 0007_39-lysA; 0007-39_BL1631; 0007_39-cg1383 |
| 132568 | 0007_39-zwf; DSS_084; 1860-pyc; DSS_033; DSS_316; 0007_39-lysA; 0007_39-cg1383 |
| 134579 | 0007_39-zwf; DSS_084; 1860-pyc; DSS_033; DSS_316; aceE_ATG > TTG; lisa68; opcA_P4; |
| | 0007_39-cg1383 |
| 134603 | 0007_39-zwf; DSS_084; 1860-pyc; DSS_033; DSS_316; 0007_39-lysA; opcA_P4; 0007_39- |
| | cg1383 |
| 144042 | 0007_39-zwf; DSS_084; 1860-pyc; DSS_033; DSS_316; aceE_ATG > TTG; lisa_68; DSS_121; |
| | 0007_39-cg1383 |
| 144051 | 0007_39-zwf; DSS_084; 1860-pyc; DSS_033; DSS_316; aceE_ATG > TTG; lisa_68; DSS_422; |
| | 0007_39-cg1383 |
| 148418 | 0007_39-zwf; DSS_084; 1860-pyc; DSS_033; fumC_ATG > GTG; opcA_P4; 1860-aceF; |
| | 0007_39-CAA; 0007_39-lysA; 0007_39-cg1383 |
| 148431 | 0007_39-zwf; DSS_084; 1860-pyc; DSS_033; fumC_ATG > GTG; opcA_P4; 1860-aceF; |
| | 0007_39-lysA; 0007_39-SCO; 0007_39-cg1383 |
| 148999 | 0007_39-zwf; DSS_084; 1860-pyc; DSS_033; DSS_316; 0007_39-lysA; 0007_39-lpd; |
| | opcA_P4; 0007_39-rpoB; 0007_39-cg1383 |
| 149034 | 0007_39-zwf; DSS_084; 1860-pyc; DSS_033; DSS_316; 0007_39-lysA; 0007_39-lpd; |
| | 0007_39-CAA; 0007_39-cg1383 |
| 149042 | 0007_39-zwf; DSS_084; 1860-pyc; DSS_033; DSS_316; 0007_39-lysA; opcA_P4; lisa_68; |
| | 0007_39-cg1383 |
| 149058 | 0007_39-zwf; DSS_084; 1860-pyc; DSS_033; DSS_316; 0007_39-lysA; 1860-xerD; 0007_39- |
| | cg1383 |
| 151595 | 0007_39-zwf; DSS_084; 1860-pyc; DSS_033; DSS_316; aceE_ATG > TTG; lisa68; opcA_P4; |
| | 0007_39-cg2766; 0007_39-cg1383 |
| 151611 | 0007_39-zwf; DSS_084; 1860-pyc; DSS_033; DSS_316; aceE_ATG > TTG; lisa68; opcA_P4; |
| | 0007_39-cg2766; 0007_39-cg1383 |
| 151753 | 0007_39-zwf; DSS_084; 1860-pyc; DSS_033; DSS_316; 0007_39-lysA; 0007_39-lpd; |
| | opcA_P4; 0007_39-glf; 0007_39-cg1383 |
| 151764 | 0007_39-zwf; DSS_084; 1860-pyc; DSS_033; DSS_316; 0007_39-lysA; 0007_39-lpd; |
| | opcA_P4; 0007_39-cg2766; 0007_39-cg1383 |
| 151804 | 0007_39-zwf; DSS_084; 1860-pyc; DSS_033; DSS_316; 0007_39-lysA; opcA_P4; 0007_39- |
| | cg2766; 0007_39-cg1383 |
| 151840 | 0007_39-zwf; DSS_084; 1860-pyc; DSS_033; DSS_316; aceE_ATG > TTG; 0007_39-lysA; |
| | 0007_39-SCO; opcA_P_4; 0007_39-cg1383 |
| 151904 | 0007_39-zwf; DSS_084; 1860-pyc; DSS_033; DSS_316; 0007_39-lysA; opcA_P4; lisa_68; |
| | 0007_39-rpoB-gene-383; 0007_39-cg1383 |
| 151914 | 0007_39-zwf; DSS_084; 1860-pyc; DSS_033; DSS_316; 0007_39-lysA; opcA_P4; lisa_68; |
| | 0007_39-rpoB-gene-383; 0007_39-cg1383 |
| 151920 | 0007_39-zwf; DSS_084; 1860-pyc; DSS_033; DSS_316; 0007_39-lysA; 0007_39-cg2766; |
| | opcA_P4; 0007_39-cg1383 |
| 151965 | 0007_39-zwf; DSS_084; 1860-pyc; DSS_033; DSS_316; aceE_ATG > TTG; lisa68; opcA_P4; |
| | 0007_39-actA; 0007_39-cg1383 |
| 152452 | 0007_39-zwf; DSS_084; 1860-pyc; DSS_033; DSS_316; aceE_ATG > TTG; lisa68; DSS_121; |
| | 0007_39-cg1383 |
| 152468 | 0007_39-zwf; DSS_084; 1860-pyc; DSS_033; DSS_316; 0007_39-lysA; 1860-xerD; 0007_39- |
| | cg1383 |
| 152500 | 0007_39-zwf; DSS_084; 1860-pyc; DSS_033; DSS_316; aceE_ATG > TTG; 0007_39-lysA; |
| | 0007_39-SCO; 0007_39-cg1383 |
| 154589 | 0007_39-zwf; DSS_084; 1860-pyc; DSS_033; DSS_316; 0007_39-lysA; 0007_39-lpd; |
| | opcA_P4; 1860-cg1768; glk; 0007_39-cg0725; 0007_39-cg1383 |
| 154600 | 0007_39-zwf; DSS_084; 1860-pyc; DSS_033; DSS_316; 0007_39-lysA; 0007_39-lpd; |
| | opcA_P4; 1860-cg1768; glk; 1860-cg1144; 0007_39-cg1383 |
| 155462 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg1383 |
| 155466 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg1383 |
| 155469 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg1383 |
| 162735 | 0007_39-zwf; DSS_084; 1860-pyc; DSS_033; DSS_316; 0007_39-lysA; opcA_P4; lisa_68; |
| | 0007_39-rpoB-gene-383; 0007_39-cg2766; 0007_39-cg1383 |
| 163111 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-cg1383 |
| 163167 | 0007_39-zwf; DSS_084; 1860-pyc; DSS_033; DSS_316; aceE_ATG > TTG; 0007_39-lysA; |
| | 0007_39-SCO; 0007_39-cg2766; opcA_P4; 0007_39-phoU; 0007_39-cg1383 |
| 172144 | 0007_39-zwf; DSS_084; 1860-pyc; DSS_033; DSS_316; aceE_ATG > TTG; 0007_39-lysA; |
| | 0007_39-SCO; opcA_P_4; 0007_39-cg0725; 0007_39-aroP; 0007_39-cg1383 |
| 172152 | 0007_39-zwf; DSS_084; 1860-pyc; DSS_033; DSS_316; 0007_39-lysA; 0007_39-lpd; |
| | opcA_P4; 1860-cg1768; glk; 0007_39-cg0725; 0007_39-cg2899; 0007_39-cg1383 |
| 172168 | 0007_39-zwf; DSS_084; 1860-pyc; DSS_033; DSS_316; 0007_39-lysA; 0007_39-lpd; |
| | opcA_P4; 1860-cg1768; glk; 1860-cg1144; 0007_39-cg2766; 0007_39-cg1383 |
| 174249 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-mutM2; 0007_39-cg1383 |
| 175204 | 0007_39-zwf; DSS_084; 1860-pyc; DSS_033; DSS_316; 0007_39-lysA; 0007_39-cg0725; |
| | opcA_P4; lisa68; 0007_39-nusG; 1860-xerD; 0007_39-cg1383 |
| 175787 | 0007_39-zwf; DSS_084; 1860-pyc; DSS_033; DSS_316; aceE_ATG > TTG; lisa68; opcA_P4; |
| | 0007_39-cg2766; 0007_39-actA; 1860-tkt; 0007_39-cg1383 |
| 175821 | 0007_39-zwf; DSS_084; 1860-pyc; DSS_033; DSS_316; aceE_ATG > TTG; lisa68; opcA_P4; |
| | 0007_39-cg2766; 0007_39-actA; 3381-cg1410; 0007_39-cg1383 |
| 176476 | 0007_39-zwf; DSS_084; 1860-pyc; DSS_033; DSS_316; aceE_ATG > TTG; lisa68; opcA_P4; |
| | 0007_39-cg2766; 0007_39-actA; 0007-_39-rho; 0007_39-cg1383 |
| 176484 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-cg1383 |
| 176485 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-cg1383 |
| 176545 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-gltD; 0007_39-cg1503; 0007_39-cg1383 |
| 176546 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-gltD; 0007_39-cg1503; 0007_39-cg1383 |
| 180360 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-phoU; 0007_39-cg1383 |
| 183641 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg800; 0007_39-cg1383 |
| 193331 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-cg1383 |
| 210851 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU; 0007_39-rpoB; 0007_39-kgd; |
| | 0007_39-cg1486; 1860-xerD; 0007_39-cg1383 |
| 101738 | 0007_39-zwf; DSS_084; 1860-pyc; DSS_033; DSS_316; 0007_39-lysA; 0007_39-cg3210 |
| 139570 | 0007_39-zwf; DSS_084; 1860-pyc; DSS_033; DSS_316; 0007_39-lysA; 0007_39-opca_P4; |
| | 9: 0007_39-cg3210 |
| 174360 | 0007_39-zwf; DSS_084; 1860-pyc; DSS_033; DSS_316; 0007_39-lysA@ni487; 0007_39- |
| | opca_P4; 0007_39-acta; 0007_39-cg2766; 0007_39-cg3210 |
| 279235 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-cg3210; |
| | 0007_39-cg0800; 1860-cg1768; 0007_39-cg3210 |
| 279255 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-0800; 0007_39- |
| | cg1486; 1860-NCgl1262; 0007_39-cg3210 |
| 279327 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-0800; 0007_39- |
| | cg1486; 0007_39-cg3210 |
| 279352 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-0800; 0007_39- |
| | cg1486; 0007_39-cg3210 |
| 182623 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-cg3210 |
| 151762 | 0007_39-zwf; DSS_084; 1860-pyc; DSS_033; DSS_316; 0007_39-lysA; 0007_39-lpd; |
| | opcA_P4; 0007_39-glf; 0007_39-phoU |
| 151773 | 0007_39-zwf; DSS_084; 1860-pyc; DSS_033; DSS_316; 0007_39-lysA; 0007_39-lpd; |
| | opcA_P4; 0007_39-cg2766; 0007_39-phoU |
| 151832 | 0007_39-zwf; DSS_084; 1860-pyc; DSS_033; DSS_316; aceE_ATG > TTG; lisa68; opcA_P4; |
| | 0007_39-cg2766; 0007_39-phoU |
| 151913 | 0007_39-zwf; DSS_084; 1860-pyc; DSS_033; DSS_316; 0007_39-lysA; opcA_P4; lisa_68; |
| | 0007_39-rpoB-gene-383; 0007_39-phoU |
| 151929 | 0007_39-zwf; DSS_084; 1860-pyc; DSS_033; DSS_316; 0007_39-lysA; 0007_39-cg2766; |
| | opcA_P4; 0007_39-phoU |
| 151969 | 0007_39-zwf; DSS_084; 1860-pyc; DSS_033; DSS_316; aceE_ATG > TTG; lisa68; opcA_P4; |
| | 0007_39-actA; 0007_39-phoU |
| 151978 | 0007_39-zwf; DSS_084; 1860-pyc; DSS_033; DSS_316; 0007_39-lysA; 0007_39-lpd; |
| | opcA_P4; 0007_39-rpoB; lisa_68; 0007_39-phoU |
| 152448 | 0007_39-zwf; DSS_084; 1860-pyc; DSS_033; DSS_316; aceE_ATG > TTG; 0007_39-lysA; |
| | 0007_39-SCO; 0007_39-phoU |
| 152464 | 0007_39-zwf; DSS_084; 1860-pyc; DSS_033; DSS_316; aceE_ATG > TTG; lisa68; DSS_121; |
| | 0007_39-phoU |
| 152480 | 0007_39-zwf; DSS_084; 1860-pyc; DSS_033; DSS_316; 0007_39-lysA; 1860-xerD; 0007_39- |
| | phoU |
| 152496 | 0007_39-zwf; DSS_084; 1860-pyc; DSS_033; DSS_316; 0007_39-lysA; 0007_39-lpd; |
| | opcA_P4; 0007_39-rpoB; 0007_39-phoU |
| 152592 | 0007_39-zwf; DSS_084; 1860-pyc; DSS_033; DSS_316; 0007_39-lysA; 0007_39-cg0725; |
| | opcA_P4; lisa68; 0007_39-phoU |
| 152616 | 0007_39-zwf; DSS_084; 1860-pyc; DSS_033; DSS_316; 0007_39-lysA; 0007_39-lpd; |
| | opcA_P4; 0007_39-cg2766; 0007_39-actA; 0007_39-phoU |
| 152628 | 0007_39-zwf; DSS_084; 1860-pyc; DSS_033; DSS_316; 0007_39-lysA; 0007_39-lpd; |
| | opcA_P4; 0007_39-cg2766; glk; 0007_39-phoU |
| 152636 | 0007_39-zwf; DSS_084; 1860-pyc; DSS_033; DSS_316; aceE_ATG > TTG; lisa68; opcA_P4; |
| | 0007_39-cg2766; glk_2966; 0007_39-phoU |
| 152648 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-phoU |
| 152672 | 0007_39-zwf; DSS_084; 1860-pyc; DSS_033; DSS_316; aceE_ATG > TTG; 0007_39-lysA; |
| | 0007_39-SCO; 0007_39-cg2766; opcA_P4; 0007_39-phoU |
| 155490 | 0007_39-zwf; DSS_084; 1860-pyc; DSS_033; DSS_316; 0007_39-lysA; 0007_39-cg2766; |
| | opcA_P4; 0007_39-cg1383; 0007_39-phoU |
| 155510 | 0007_39-zwf; DSS_084; 1860-pyc; DSS_033; DSS_316; 0007_39-lysA; 0007_39-cg2766; |
| | opcA_P4; 0007_39-cg1383; 0007_39-phoU |
| 155525 | 0007_39-zwf; DSS_084; 1860-pyc; DSS_033; DSS_316; 0007_39-lysA; opcA_P4; lisa_68; |
| | 0007_39-rpoB-gene-383; 0007_39-cg1383; 0007_39-phoU |
| 155545 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-phoU |
| 162721 | 0007_39-zwf; DSS_084; 1860-pyc; DSS_033; DSS_316; 0007_39-lysA; 0007_39-lpd; |
| | opcA_P4; 1860-cg1768; glk; 0007_39-cg0725; 0007_39-phoU |
| 162743 | 0007_39-zwf; DSS_084; 1860-pyc; DSS_033; DSS_316; 0007_39-lysA; opcA_P4; lisa_68; |
| | 0007_39-rpoB-gene-383; 0007_39-cg2766; 0007_39-phoU |
| 162789 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-gltD; 0007_39-phoU |
| 163154 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-gltD; 0007_39-phoU |
| 172143 | 0007_39-zwf; DSS_084; 1860-pyc; DSS_033; DSS_316; aceE_ATG > TTG; 0007_39-lysA; |
| | 0007_39-SCO; opcA_P_4; 0007_39-cg0725; 0007_39-aroP; 0007_39-phoU |
| 172151 | 0007_39-zwf; DSS_084; 1860-pyc; DSS_033; DSS_316; 0007_39-lysA; 0007_39-lpd; |
| | opcA_P4; 1860-cg1768; glk; 0007_39-cg0725; 0007_39-cg2899; 0007_39-phoU |
| 172199 | 0007_39-zwf; DSS_084; 1860-pyc; DSS_033; DSS_316; aceE_ATG > TTG; lisa68; opcA_P4; |
| | 0007_39-cg2766; 0007_39-actA; SCO_Ni2966; 0007_39-phoU |
| 172228 | 0007_39-zwf; DSS_084; 1860-pyc; DSS_033; DSS_316; 0007_39-lysA; 0007_39-lpd; |
| | opcA_P4; 0007_39-glf; 0007_39-cg1383; 0007_39-aroP; 0007_39-phoU |
| 174199 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg0725; 0007_39-phoU |
| 174232 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-phoU |
| 174240 | 0007_39-zwf; DSS_084; 1860-pyc; DSS_033; DSS_316; aceE_ATG > TTG; lisa68; opcA_P4; |
| | 0007_39-cg2766; 0007_39-actA; 1860-tkt; 0007_39-phoU |
| 174248 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-mutM2; 0007_39-phoU |
| 174264 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU |
| 174281 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-phoU |
| 175029 | 0007_39-zwf; DSS_084; 1860-pyc; DSS_033; DSS_316; aceE_ATG > TTG; lisa68; opcA_P4; |
| | 0007_39-cg2766; 0007_39-actA; 0007-_39-rho; 0007_39-phoU |
| 175046 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 0007_39-phoU |
| 175062 | 0007_39-zwf; DSS_084; 1860-pyc; DSS_033; DSS_316; aceE_ATG > TTG; lisa68; opcA_P4; |
| | 0007_39-cg2766; 0007_39-actA; 1860-tkt; 0007_39-phoU |
| 175086 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU |
| 175102 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-phoU |
| 175165 | 0007_39-zwf; DSS_084; 1860-pyc; DSS_033; DSS_316; aceE_ATG > TTG; lisa68; opcA_P4; |
| | 0007_39-actA; 0007_39-cg1383; 3121-prpB28; 0007_39-phoU |
| 175181 | 0007_39-zwf; DSS_084; 1860-pyc; DSS_033; DSS_316; 0007_39-lysA; 0007_39-lpd; |
| | opcA_P4; 1860-cg1768; glk; 0007_39-cg0725; 0007_39-cg2766; 0007_39-phoU |
| 175183 | 0007_39-zwf; DSS_084; 1860-pyc; DSS_033; DSS_316; 0007_39-lysA; 0007_39-cg0725; |
| | opcA_P4; lisa68; 0007_39-actA; 0007_39-nusG; 0007_39-phoU |
| 175184 | 0007_39-zwf; DSS_084; 1860-pyc; DSS_033; DSS_316; 0007_39-lysA; 0007_39-cg0725; |
| | opcA_P4; lisa68; 0007_39-actA; 0007_39-nusG; 0007_39-phoU |
| 175200 | 0007_39-zwf; DSS_084; 1860-pyc; DSS_033; DSS_316; 0007_39-lysA; 0007_39-cg0725; |
| | opcA_P4; lisa68; 0007_39-nusG; 1860-xerD; 0007_39-phoU |
| 175816 | 0007_39-zwf; DSS_084; 1860-pyc; DSS_033; DSS_316; aceE_ATG > TTG; lisa68; opcA_P4; |
| | 0007_39-cg2766; 0007_39-actA; 3381-cg1410; 0007_39-phoU |
| 176509 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU |
| 176542 | 0007_39-zwf; DSS_084; 1860-pyc; DSS_033; DSS_316; aceE_ATG > TTG; lisa68; opcA_P4; |
| | 0007_39-cg2766; 0007_39-actA; 3121-gltD; 0007_39-cg1503; 0007_39-phoU |
| 183629 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-phoU |
| 186920 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg0800; 0007_39-phoU |
| 187910 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800; |
| | 0007_39-phoU |
| 187918 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-kgd; 0007_39-cg0800; 0007_39- |
| | phoU |
| 187926 | 0007_39-zwf; DSS_084; 1860-pyc; DSS_033; DSS_316; aceE_ATG > TTG; lisa68; opcA_P4; |
| | 0007_39-actA; 0007_39-cg1383; Pcg0007-amtB; 0007_39-kgd; 0007_39-cg0800; 0007_39- |
| | phoU |
| 187934 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 3121-cg1081; 0007_39-cg0800; 0007_39-phoU |
| 187942 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU;0007_39-cg0800; |
| | 0007_39-phoU |
| 187990 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-phoU |
| 188149 | 0007_39-zwf; DSS_084; 1860-pyc; DSS_033; DSS_316; 0007_39-lysA; 0007_39-lpd; |
| | opcA_P4; 0007_39-glf; 0007_39-cg1383; 0007_39-aroP; 0007_39-kgd; 0007_39-phoU |
| 188159 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-kgd; 0007_39- |
| | phoU |
| 190101 | 0007_39-zwf; DSS_084; 1860-pyc; DSS_033; DSS_316; aceE_ATG > TTG; lisa68; opcA_P4; |
| | 0007_39-cg2766; 0007_39-actA; 0007_39-cg800; 0007_39-cg2899; 0007_39-phoU |
| 193273 | 0007_39-zwf; DSS_084; 1860-pyc; DSS_033; DSS_316; 0007_39-lysA; 0007_39-lpd; |
| | opcA_P4; 0007_39-glf; 0007_39-cg1383; 0007_39-aroP; 0007_39-kgd; 0007_39-phoU |
| 193289 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800; |
| | 0007_39-phoU |
| 193293 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800; |
| | 0007_39-phoU |
| 196697 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-phoU |
| 197877 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800; |
| | 0007_39-cg1486; 0007_39-phoU |
| 197885 | 0007_39-zwf; DSS_084; 1860-pyc; DSS_033; DSS_316; aceE_ATG > TTG; lisa68; opcA_P4; |
| | 0007_39-cg2766; 0007_39-actA; glk_2966; 3121-cg1081; 0007_39-cg0800; 0007_39- |
| | ncgl1511; 0007_39-phoU |
| 197954 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-Ncgl0304; 0007_39-phoU |
| 201612 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-cg1486; 0007_39-phoU |
| 201628 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; 0755- |
| | dapA; 0007_39-phoU |
| 201644 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-Ncgl0767; 0007_39-phoU |
| 201660 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-NCgl1262; 0007_39-phoU |
| 201676 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800; |
| | 0007_39-NCgl1262; 0007_39-phoU |
| 206820 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; |
| | 0007_39-NCgl1262; 0007_39-phoU |
| 206860 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-ureR; 0007_39-cg0800; 0755- |
| | dapA; 0007_39-phoU |
| 212930 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-rpoB; 0007_39-cg0800; |
| | 0007_39-cg1486; 0007_39-glpX; 0007_39-phoU |
| 212954 | 0007_39-zwf; DSS_084; 1860-pyc; DSS_033; DSS_316; aceE_ATG > TTG; lisa68; opcA_P4; |
| | 0007_39-actA; 0007_39-cg1383; 0007_39-cg2766; 0007_39-phoU; 0007_39-cg1486; |
| | 0007_39-cg0800; 0007_39-phoU |

TABLE 8
Sequence Listing

| SEQ ID NO: | sequence |
|---|---|
| 1 | C. glutamicum NRRL B-11474, RnaJ homology region |
| 2 | coding sequence for altered RnaJ protein |
| 3 | altered RnaJ protein |
| 4 | C. glutamicum NRRL B-11474, accDA homology region |
| 5 | coding sequence for altered accDA protein |
| 6 | altered accDA protein |
| 7 | C. glutamicum NRRL B-11474, cg1144 homology region |
| 8 | coding sequence for altered cg1144 protein |
| 9 | altered cg1144 protein |
| 10 | C. glutamicum NRRL B-11474, pyc homology region |
| 11 | promoter 1860 |
| 12 | promoter 1860 and pyc coding sequence |
| 13 | C. glutamicum NRRL B-11474, aceE homology region |
| 14 | altered aceE coding sequence |
| 15 | C. glutamicum NRRL B-11474, lysA insertion homology region |
| 16 | codon-optimized lysA coding sequence |
| 17 | promoter cg0007_39-codon-optimized lysA coding sequence-sod |
| | terminator |
| 18 | C. glutamicum NRRL B-11474, lysA |
| 19 | C. glutamicum NRRL B-11474 zwf homology region |
| 20 | Promoter cg0007_39 |
| 21 | Promoter cg0007_39 insertion and zwf coding region |
| 22 | C. glutamicum NRRL B-11474, opcA homology region |
| 23 | C. humireducens opcA coding sequence |
| 24 | promoter cg0007_39-C. humireducense opcA coding sequence |
| 25 | C. humireducens opcA protein |
| 26 | C. glutamicum NRRL B-11474, cg2766 homology region |
| 27 | Promoter cg0007_39-cg2766 |
| 28 | C. glutamicum NRRL B-11474, actA homology region |
| 29 | promoter cg0007_39-actA |
| 30 | primer FarI 3p InfusF |
| 31 | primer FarI 3p InfusR AvrII |
| 32 | primer FarI 5p InfusF |
| 33 | primer FarI 5p Infus R |
| 34 | primer Pcg0007_39-zwf_P1 |
| 35 | primer Pcg0007_39-zwf_P2 |
| 36 | primer Pcg0007_39-zwf_P3 |
| 37 | primer Pcg0007_39-zwf_P4 |
| 38 | primer SNP_084_P1 |
| 39 | primer SNP_084_P2 |
| 40 | primer SNP_084_P3 |
| 41 | primer SNP_084_P4 |
| 42 | primer Pcg1860_pyc_P1 |
| 43 | primer Pcg1860_pyc_P2 |
| 44 | primer Pcg1860_pyc_P3 |
| 45 | primer Pcg1860_pyc_P4 |
| 46 | primer SNP_033_P1 |
| 47 | primer SNP_033_P2 |
| 48 | primer SNP_033_P3 |
| 49 | primer SNP_033_P4 |
| 50 | primer SNP_316_P1 |
| 51 | primer SNP_316_P2 |
| 52 | primer SNP_316_P3 |
| 53 | primer SNP_316_P4 |
| 54 | primer SCS_aceE_P1 |
| 55 | primer SCS_aceE_P2 |
| 56 | primer SCS_aceE_P3 |
| 57 | primer SCS_aceE_P4 |
| 58 | primer opcA_P1 |
| 59 | primer opcA_P2 |
| 60 | primer opcA_P3 |
| 61 | primer opcA_P4 |
| 62 | primer pcg0007_39-cg2766_p1 |
| 63 | primer pcg0007_39-cg2766_P2 |
| 64 | primer pcg0007_39-cg2766_P3 |
| 65 | primer pcg0007_39-cg2766_P4 |
| 66 | primer pcg0007_39-actA_P1 |
| 67 | primer pcg0007_39-actA_P2 |
| 68 | primer pcg0007_39-actA_P3 |
| 69 | primer pcg0007_39-actA_P4 |
| 70 | primer 184f7 |
| 71 | primer 184F8 |
| 72 | primer 244-4Go F2 Inf SpeI |
| 73 | primer 253-4Go R3 |
| 74 | primer 685 |
| 75 | primer 686 |
| 76 | primer 687 |
| 77 | primer 693 |
| 78 | C. glutamicum RnaJ protein |
| 79 | C. glutamicum RnaJ protein |
| 80 | C. ulcerans RnaJ protein |
| 81 | C. glutamicum accDA protein |
| 82 | C. glutamicum MB001 accDA protein |
| 83 | C. crudilactis accDA protein |
| 84 | C. glutamicum cg1144 protein |
| 85 | C. crudilactis cg1144 protein |
| 86 | C. efficiens YS-314cg1144 protein |
| 87 | C. vitaeruminis DSM 20294 opcA protein |
| 88 | C. matruchotii ATCC 33806 opcA protein |
| 89 | C. falsenii DSM 44353 opcA protein |
| 90 | C. halotolerans YIM 70093 = DSM 44683 opcA protein |
| 91 | C. pyruviciproducens ATCC BAA-1742 opcA protein |
| 92 | C. casei UCMA 3821 opcA protein |
| 93 | C. terpenotabidum Y-11 opcA protein |
| 94 | C. glutamicum NRRL B-11474 opcA protein |
| 95 | promoter 3121 |
| 96 | promoter 0755 |
| 97 | promoter 3381 |
| 98 | C. glutamicum cg1383 homology region |
| 99 | primer Pcg0007_39-cg1383_P1 |
| 100 | primer Pcg0007_39-cg1383_P2 |
| 101 | primer Pcg0007_39-cg1383_P3 |
| 102 | primer Pcg0007_39-cg1383_P4 |
| 103 | Pcg0007_39 promoter inserted in front of cg1383 start codon |
| 104 | C. glutamicum phoU homology region |
| 105 | primer Pcg0007_39-phoU_P1 |
| 106 | primer Pcg0007_39-phoU_P2 |
| 107 | primer Pcg0007_39-phoU_P3 |
| 108 | primer Pcg0007_39-phoU_P4 |
| 109 | Pcg0007_39 promoter in front of phoU start codon |
| 110 | C. glutamicum cg3210 homology region |
| 111 | Pcg0007_39-cg3210-p1 |
| 112 | Pcg0007_39-cg3210-p2 |
| 113 | Pcg0007_39-cg3210-p3 |
| 114 | Pcg0007_39-cg3210-p4 |
| 115 | Pcg0007_39 in front of cg3210 |
| 116 | C. glutamicum cg0800 homology region |
| 117 | Pcg0007_39-cg0800-p1 |
| 118 | Pcg0007_39-cg0800-p2 |
| 119 | Pcg0007_39-cg0800-p3 |
| 120 | Pcg0007_39-cg0800-p4 |
| 121 | Pcg0007_39 in front of cg0800 |

### REFERENCES

Becker et al., “Systems level engineering of Corynebacterium glutamicum—Reprogramming translational efficiency for superior production,” Engineering in Life Sciences 10, 430-38, 2010
Buchholz et al., “Platform Engineering of Corynebacterium glutamicum with Reduced Pyruvate Dehydrogenase Complex Activity for Improved Production of 1-Lysine, 1-Valine, and 2-Ketoisovalerate,” Applied Env. Microbiol. 79, 5566-75, 2013
Knoppová et al., Curr. Microbiol. 55, 234-39, 2007
Lee, J Microbiol Biotechnol. 2014 January; 24(1):70-9
Nešvera et al., Subcell. Biochem. 2012; 64:203-21
Pátek & Nešvera, “Promoters and Plasmid Vectors of Corynebacterium glutamicum,” in Corynebacterium glutamicum, Yukawa & Inui, eds., Springer-Verlag, Berlin 2013, pp. 51-88
Pátek et al., J. Biotechnol. 104, 311-23, 2003(a)
Pátek et al., J. Biotechnol. 104, 325-34, 2003(b)
Pátek et al., Microb. Biotechnol. 2013 March; 6(2):103-17
Pfeifer-Sancar et al., BMC Genomics 14, 888, 2013
Rytter et al., Appl. Microbiol Biotechnol. 2014 March; 98(6):2617-23
Shang et al., Biotechnol. Lett. 2017 Nov. 21. doi: 10.1007/s10529-017-2479-y
Yim et al., Biotechnol. Bioeng. 2013 November; 110(11):2959-69

1. The Corynebacterium deposited as NRRL B-67535.

2. A Corynebacterium having a bacterial genome consisting essentially of the genome of the bacterium deposited as NRRL B-67439 but for up to 4 structural alterations selected from the group consisting of:
(a) insertion of a promoter in front of the cg1383 start codon;
(b) replacement of the native phoU promoter;
(c) replacement of the native cg3210 promoter; and
(d) replacement of the native cg0800 promoter.

3. (canceled)

4. A method of producing lysine, comprising:
(a) culturing a Corynebacterium; and
(b) recovering lysine produced by the Corynebacterium,
wherein the Corynebacterium is selected from the group consisting of:
(i) a Corynebacterium deposited as NRRL B-67535; and
(ii) a Corynebacterium having a bacterial genome consisting essentially of the genome of the bacterium deposited as NRRL B-67439 but for up to 4 structural alterations selected from the group consisting of:
(1) insertion of a promoter in front of the cg1383 start codon;
(2) replacement of the native phoU promoter;
(3) replacement of the native cg3210 promoter; and
(4) replacement of the native cg0800 promoter.

5. The Corynebacterium of claim 2, which comprises the structural alteration of the promoter inserted in front of the cg1383 start codon.

6. The Corynebacterium of claim 2, which comprises the replacement of the native phoU promoter.

7. The Corynebacterium of claim 2, which comprises the replacement of the native cg3210 promoter.

8. The Corynebacterium of claim 2, which comprises the replacement of the native cg0800 promoter.

9. The method of claim 4, wherein the Corynebacterium is the Corynebacterium deposited as NRRL B-67535.

10. The method of claim 4, wherein the Corynebacterium has the bacterial genome consisting essentially of the genome of the bacterium deposited as NRRL B-67439 but for the up to 4 structural alterations.

11. The method of claim 10, wherein a promoter is inserted in front of the cg1383 start codon.

12. The method of claim 10, wherein the native phoU promoter is replaced.

13. The method of claim 10, wherein the native cg3210 promoter is replaced.

14. The method of claim 10, wherein the native cg0800 promoter is replaced.
